(19) United States
(12) Patent Application Publication
(10) Pub. No.: US 2025/0161488 A1
(43) Pub. Date: May 22, 2025

(54) THERAPEUTIC mRNA

(71) Applicant: The University of Melbourne, Melbourne (AU)

(72) Inventors: Denzil FURTADO, Melbourne (AU); Frank CARUSO, Melbourne (AU)

(21) Appl. No.: 18/833,748

(22) PCT Filed: Jan. 27, 2023
(86) PCT No.: PCT/AU2023/050048

§ 371 Date: Jul. 26, 2024

(30) Foreign Application Priority Data
Jan. 28, 2022 (AU) 2022900150

(51) Int. Cl. A61K 48/00; C07K 14/47; G01N 33/68

(52) U.S. Cl. CPC A61K 48/005; C07K 14/47; G01N 33/68

(57) ABSTRACT

The present invention relates to mRNA, mRNA compositions, kits and uses thereof for the treatment of lysosomal storage diseases. In one aspect, the present invention relates to the treatment of Niemann-Pick type C disease.

### CROSS-REFERENCE TO RELATED APPLICATIONS

This application claims priority to Australian Provisional Patent Application 2022900150, filed on 28 Jan. 2022, the entire contents of which is hereby incorporated by reference in its entirety.

### FIELD OF THE INVENTION

The present invention relates to mRNA, mRNA compositions, kits and uses thereof for the treatment of lysosomal storage diseases. In one aspect, the present invention relates to the treatment of Niemann-Pick type C disease.

### BACKGROUND OF THE INVENTON

Lysosomal storage diseases (LSDs) are a group of inherited metabolic disorders that are predominantly caused by enzyme deficiencies within the lysosome resulting in accumulation of undegraded substrate. This leads to a broad spectrum of clinical manifestations depending on the specific substrate and site of accumulation. Examples of LSDs include the mucopolysaccharidoses, mucolipidoses, oligosaccharidoses, Pompe disease, Gaucher disease, Fabry disease and neuronal ceroid lipofuscinoses.

Another example of a LSD is Niemann-Pick type C (NPC) disease. This disease is a rare inherited neurovisceral disorder that arises due to inactivating mutations in one of two protein-coding genes, NPC1 (95% of cases) or NPC2 (Vanier et al (2010)). The incidence of NPC1 disease is approximately 1/90,000 live births, but due to the extreme heterogeneity of clinical phenotypes, a late-onset form of NPC1 is estimated to have a much higher incidence rate of between 1/19,000-1/36,000 (Wassif et al (2016)). Both NPC1 and NPC2 are endo-lysosomal proteins that, under normal circumstances, act cooperatively to shuttle unesterified cholesterol obtained from endocytosed lipoproteins across the late endosomal membrane into the cytosol (Subramanian et al (2008)). NPC2, a small, soluble protein, binds unesterified cholesterol in the lumen and presents it to the N-terminal domain of NPC1 (Hoglonger et al (2019); Sleat etl al (2004)). NPC1, which by contrast is a large, multipass transmembrane protein, subsequently facilitates cholesterol egress across the endosomal membrane (Hoglonger et al (2019); Kwon et al (2009)).

In NPC disease, defective trafficking leads to the sequestration and accumulation of unesterified cholesterol and other lipids including glycosphingolipids in lysosomes of cells throughout the body. This results in damage to various organs including the central nervous system (CNS), liver, spleen, and the lungs (Vanier et al (2010)). The clinical manifestations of these pathologies often arise at different times and follow very different trajectories (Patterson et al 2012)). At opposite ends of the spectrum, some severe infantile forms of the disease result in fatal liver or respiratory failure (Patterson et al 2012), while other adult forms result in late-onset neurodegenerative disease (Wassif et al (2016)). Nevertheless, classical neurological manifestations in the majority of patients include vertical supranuclear gaze palsy, cerebellar ataxia, dystonia, dysarthria, and dysphagia, gelastic cataplexy, and epileptic seizures (Vanier et al (2010)); Wassif et al ((2016)); Patterson et al (2012)). The severity of these symptoms tends to worsen with time, as NPC disease is chronic, progressive, and invariably fatal (Yanjanin et al (2010)).

Despite many and varied efforts, few treatments for NPC disease have been approved for use (Hammon et al (2019)). No drug has yet been approved by the United States (US) Food and Drug Administration (FDA), although various compounds have been introduced into late-stage clinical trials, including arimoclomol, an inducer of heat shock proteins (ClinicalTrials.gov identifier NCT02612129) (Kirkgaard et al (2016)); miglustat, a glucosylceramide synthase inhibitor (ClinicalTrials.gov identifier NCT01760564) (Patterson et al (2020)); Solomon et al (2020)); and hydroxypropyl-beta-cyclodextrin, a cyclic oligosaccharide that has been shown to sequester and transport unesterified cholesterol from the late endosome to the cytosol, thereby enabling increased cholesterol esterification (ClinicalTrials.gov identifier NCT04860960) (Liu et al (2009)). Miglustat is currently approved in the EU, Canada and Japan for the treatment of progressive neurological complications in NPC disease, and is frequently prescribed off-label for NPC1 disease in the US (Solomon et al 2020). Hydroxypropyl-beta-cyclodextrin, meanwhile, has been approved under compassionate use protocols to treat several NPC disease patients worldwide since 2009 (Hastings et al (2019)). Even with the aforementioned drugs, however, the prognosis for patients remains poor and the disease burden remains high.

Since NPC disease occurs due to a lack of functional NPC1 or NPC2 protein, therapeutic approaches that introduce a functional copy of the affected protein have been explored. Protein replacement therapy is not available for NPC1 since it is a transmembrane protein, but has demonstrated partial efficacy in a mouse model of NPC2 disease (Nielson et al (2011)). Viral gene therapy strategies, reliant on adeno-associated viral vector serotype 9 (AAV9), have shown promise in mouse models of NPC disease when administered systemically (Chandler et al (2017)) and locally (Hughes et al (2018); Xie et al (2017)). However, AAV gene therapy has also been associated with some important drawbacks, including the risk of insertional mutagenesis, potentially-fatal liver toxicity, and strong immunogenicity resulting in an inability to redose (Li et al (2021); Verdera et al (2020)).

In view of the above described limitations, there is a need for development of alterantive strategies for the treatment of lysosomal storage diseases including Niemann-Pick type C disease.

### SUMMARY OF THE INVENTION

The present inventors demonstrate for the first time that delivery of exogenous mRNA containing a chemical modification and/or codon optimisation can be used for the treatment of lysosomal storage diseases, including Niemann-Pick type C disease. In particular, restoration of functional protein expression has been achieved for large proteins, including NPC-1. This has significant implications for the treatment of lysosomal storage diseases, including Niemann-Pick type C disease.

In an aspect of the invention, there is therefore provided a messenger RNA (mRNA) encoding a therapeutic polypeptide, wherein the mRNA comprises one or both of:
(i) an optimised codon;
(ii) a chemical modification,
   when compared to a corresponding mRNA that does not comprise the optimised codon and/or chemical modification. Preferably, the mRNA comprises an optimised codon and a chemical modification. More preferably, the chemical modification is a uracil nucleoside modification, optionally modified to M1ψTP or 5moUTP, and the optimised codon comprises substituting adenine (A) or uracil (U) containing codons with codons enriched in guanine (G) or cytosine (C), optionally at the third base of the codon.

In another aspect of the invention, there is provided a messenger RNA (mRNA) encoding an intracellular lipid trafficker, wherein the mRNA comprises one or both of:
(i) an optimised codon;
(ii) a chemical modification,
   when compared to a corresponding mRNA that does not comprise the optimised codon and/or chemical modification.

In an embodiment, the intracellular lipid trafficker functions within a lysosome of a cell.

In an embodiment, the mRNA encodes an intracellular cholesterol trafficker selected from NPC intracellular cholesterol transporter 1 (NPC-1) and NPC intracellular cholesterol transporter 2 (NPC-2), preferably NPC-1.

In an embodiment, the mRNA encoding NPC-1 comprises a sequence having at least 50%, at least 60%, at least 70%, at least 75%, at least 80%, at least 85%, at least 90%, at least 91%, at least 92%, at least 93%, at least 94%, at least 95%, at least 96%, at least 97%, at least 98%, at least 99%, at least 99.5% or at least 99.8% identity to a sequence set forth as SEQ ID NO: 2 or 8. In a preferred embodiment, the mRNA encoding NPC-1 comprises a sequence set forth as SEQ ID NO: 2.

In an embodiment, the mRNA encoding NPC-2 comprises a sequence having at least 50%, at least 60%, at least 70%, at least 75%, at least 80%, at least 85%, at least 90%, at least 91%, at least 92%, at least 93%, at least 94%, at least 95%, at least 96%, at least 97%, at least 98%, at least 99%, at least 99.5% or at least 99.8% identity to a sequence set forth as SEQ ID NO: 9.

In an embodiment, the lipid comprises cholesterol. Preferably, the cholesterol is unesterified cholesterol. In yet another embodiment, the lipid comprises sphingomyelin, glycosphingolipid or bis(monoacylglycerol) phosphate. In an embodiment, the glycosphingolipid includes cerebrosides, gangliosides and globosides.

In an embodiment, the mRNA comprises an optimised codon and a chemical modification. Preferably, the chemical modification is a nucleoside modification, preferably a uracil or cytosine modification.

In an embodiment, the chemical modification increases mRNA stability and/or mRNA translation when compared to a mRNA without the chemical modification.

In an embodiment, the uracil modification is selected from the group consisting of pseudouridine (ψ), pyridin-4-one ribonucleoside, 5-aza-uridine, 6-aza-uridine, 2-thio-5-aza-uridine, 2-thio-uridine (s²U), 4-thio-uridine (s⁴U), 4-thiopseudouridine, 2-thio-pseudouridine, 5-hydroxy-uridine (ho⁵U), 5-aminoallyl-uridine, 5-halo-uridine (e.g., 5-iodo-uridine or 5-bromo-uridine), 3-methyluridine (m³U), 5-methoxy-uridine (mo⁵U), 5-methoxy-uridine triphosphate (5moUTP), uridine 5-oxyacetic acid (cmo⁵U), uridine 5-oxyacetic acid methyl ester (mcmo⁵U), 5-carboxymethyl-uridine (cm⁵U), 1-carboxymethyl-pseudouridine, 5-carboxyhydroxymethyl-uridine (chm⁵U), 5-carboxyhydroxymethyl-uridine methyl ester (mchm⁵U), 5-methoxycarbonylmethyl-uridine (mcm⁵U), 5-methoxycarbonylmethyl-2-thio-uridine (mcm⁵s²U), 5-aminomethyl-2-thio-uridine (nm⁵s²U), 5-methylaminomethyl-uridine (mnm⁵U), 5-methylaminomethyl-2-thio-uridine (mnm⁵s²U), 5-methylaminomethyl-2-seleno-uridine (mnm⁵se²U), 5-carbamoylmethyl-uridine (ncm⁵U), 5-carboxymethylaminomethyl-uridine (cmnm⁵U), 5-carboxymethylaminomethyl-2-thio-uridine (cmnm⁵s²U), 5-propynyluridine, 1-propynyl-pseudouridine, 5-taurinomethyl-uridine (tm⁵U), 1-taurinomethyl-pseudouridine, 5-taurinomethyl-2-thio-uridine (tm⁵s²U), 1-taurinomethyl-4-thio-pseudouridine, 5-methyl-uridine (m⁵U, i.e., having the nucleobase deoxythymine), 1-methylpseudouridine (m¹ψ), 5-methyl-2-thiouridine (m⁵s²U), 1-methyl-4-thio-pseudouridine (m¹s⁴ψ), 4-thio-1-methyl-pseudouridine, 3-methyl-pseudouridine (m³ψ), 2-thio-1-methyl-pseudouridine, 1-methyl-1-deaza-pseudouridine, 2-thio-1-methyl-1-deaza-pseudouridine, dihydrouridine (D), dihydropseudouridine, 5,6-dihydrouridine, 5-methyldihydrouridine (m⁵D), 2-thio-dihydrouridine, 2-thio-dihydropseudouridine, 2-methoxy-uridine, 2-methoxy-4-thio-uridine, 4-methoxy-pseudouridine, 4-methoxy-2-thio-pseudouridine, N1-methyl-pseudouridine (also known as 1-methylpseudouridine (m¹ψ), 3-(3-amino-3-carboxypropyl) uridine (acp³U), 1-methyl-3-(3-amino-3-carboxypropyl) pseudouridine (acp³ψ), 5-(isopentenylaminomethyl) uridine (inm⁵U), 5-(isopentenylaminomethyl)-2-thio-uridine (inm⁵s²U), a-thio-uridine, 2′-O-methyl-uridine (Um), 5,2′-O-dimethyl-uridine (m⁵Um), 2′-O-methyl-pseudouridine (ψm), 2-thio-2′-O-methyl-uridine (s²Um), 5-methoxycarbonylmethyl-2′-O-methyl-uridine (mcm⁵Um), 5-carbamoylmethyl-2′-O-methyl-uridine (ncm⁵Um), 5-carboxymethylaminomethyl-2′-O-methyluridine (cmnm⁵Um), 3,2′-O-dimethyl-uridine (m³Um), 5-(isopentenylaminomethyl)-2′-O-methyl-uridine (inm⁵Um), 1-thio-uridine, deoxythymidine, 2′-F-arauridine, 2′-F-uridine, 2′-OH-ara-uridine, 5-(2-carbomethoxyvinyl) uridine, and 5-[3-(1-E-propenylamino)]uridine. Preferably, the uracil modification is 5-methoxyuridine triphosphate (5moUTP) or N1-methylpseudouridine 5′-triphosphate (M1ψTP), more preferably M1ψTP.

In an embodiment, at least 10%, at least 20%, at least 30%, at least 40%, at least 45%, at least 50%, at least 55%, at least 60%, at least 65%, at least 70%, at least 75%, at least 80%, at least 85%, at least 90%, at least 95%, at least or 100% of uracils are modified.

In an embodiment, at least 10%, at least 20%, at least 30%, at least 40%, at least 45%, at least 50%, at least 55%, at least 60%, at least 65%, at least 70%, at least 75%, at least 80%, at least 85%, at least 90%, at least 95%, at least or 100% of uracils are modified to 5-methoxyuridine triphosphate (5moUTP) or N1-methylpseudouridine 5′-triphosphate (M1ψTP), preferably M1ψTP.

In an embodiment, each uracil of a coding region of a mRNA of the invention is modified to 5-methoxyuridine triphosphate (5moUTP) or N1-methylpseudouridine 5′-triphosphate (M1ψTP), preferably M1ψTP.

In another embodiment, the nucleoside modification is a modified cytosine. In this embodiment, the cytosine modification may be selected from the group consisting of 5-aza-cytidine, 6-aza-cytidine, pseudoisocytidine, 3-methyl-cytidine (m³C), N4-acetyl-cytidine (ac⁴C), 5-formyl-cytidine (f⁵C), N4-methyl-cytidine (m⁴C), 5-methyl-cytidine (m⁵C), 5-methy-lcytidine 5′-triphosphate (5mCTP), 5-halo-cytidine (e.g., 5-iodo-cytidine), 5-hydroxymethyl-cytidine (hm⁵C), 1-methyl-pseudoisocytidine, pyrrolo-cytidine, pyrrolo-pseudoisocytidine, 2-thio-cytidine (s²C), 2-thio-5-methyl-cytidine, 4-thio-pseudoisocytidine, 4-thio-1-methyl-pseudoisocytidine, 4-thio-1-methyl-1-deaza-pseudoisocytidine, 1-methyl-1-deaza-pseudoisocytidine, zebularine, 5-aza-zebularine, 5-methyl-zebularine, 5-aza-2-thio-zebularine, 2-thiozebularine, 2-methoxy-cytidine, 2-methoxy-5-methyl-cytidine, 4-methoxy-pseudoisocytidine, 4-methoxy-1-methyl-pseudoisocytidine, lys idine (k₂C), a-thiocytidine, 2′-O-methyl-cytidine (Cm), 5,2′-O-dimethyl-cytidine (m⁵Cm), N4-acetyl-2′-O-methyl-cytidine (ac⁴Cm), N4,2′-O-dimethyl-cytidine (m⁴Cm), 5-formyl-2′-O-methyl-cytidine (f⁵Cm), N4,N4,2′-O-trimethyl-cytidine (m⁴₂Cm), 1-thio-cytidine, 2′-F-ara-cytidine, 2′-F-cytidine, and 2′-OH-ara-cytidine. Preferably, the the nucleoside modification is 5-methylcytidine 5′-triphosphate (5mCTP).

In an embodiment, at least 10%, at least 20%, at least 30%, at least 40%, at least 45%, at least 50%, at least 55%, at least 60%, at least 65%, at least 70%, at least 75%, at least 80%, at least 85%, at least 90%, at least 95%, at least or 100% of cytosines are modified.

In an embodiment, at least 10%, at least 20%, at least 30%, at least 40%, at least 45%, at least 50%, at least 55%, at least 60%, at least 65%, at least 70%, at least 75%, at least 80%, at least 85%, at least 90%, at least 95%, at least or 100% of cytosines are modified to 5-methylcytidine 5′-triphosphate (5mCTP).

In an embodiment, the optimised codon increases mRNA stability and/or mRNA translation when compared to a mRNA without the optimised codon. In another embodiment, the optimised codon increases guanine (G) and/or cytosine (C) codon content. Preferably, the optimised codon comprises substituting adenine (A) or uracil (U) containing codons with codons enriched in guanine (G) or cytosine (C), preferably at the third base of a codon.

In an embodiment, at least 10%, at least 20%, at least 30%, at least 40%, at least 45%, at least 50%, at least 55%, at least 60%, at least 65%, at least 70%, at least 75%, at least 80%, at least 85%, at least 90%, at least 95%, at least or 100% of codons are modified.

In an embodiment, at least 10%, at least 20%, at least 30%, at least 40%, at least 45%, at least 50%, at least 55%, at least 60%, at least 65%, at least 70%, at least 75%, at least 80%, at least 85%, at least 90%, at least 95%, at least or 100% of codons are modified to increase guanine (G) and/or cytosine (C) codon content.

In an embodiment, at least 10%, at least 20%, at least 30%, at least 40%, at least 45%, at least 50%, at least 55%, at least 60%, at least 65%, at least 70%, at least 75%, at least 80%, at least 85%, at least 90%, at least 95%, at least or 100% of adenine (A) or uracil (U) containing codons are substituted with codons enriched in guanine (G) or cytosine (C), preferably at the third base of a codon.

In an embodiment of the invention, at least 10%, at least 20%, at least 30%, at least 40%, at least 45%, at least 50%, at least 55%, at least 60%, at least 65%, at least 70%, at least 75%, at least 80%, at least 85%, at least 90%, at least 95%, at least or 100% of uracil of a coding region of a mRNA of the invention is modified to M1ψTP and at least 10%, at least 20%, at least 30%, at least 40%, at least 45%, at least 50%, at least 55%, at least 60%, at least 65%, at least 70%, at least 75%, at least 80%, at least 85%, at least 90%, at least 95%, at least or 100% of codons comprising A or U, preferably at the third base, are substituted with codons enriched in guanine (G) or cytosine (C).

In a preferred embodiment of the invention, uracil of a coding region of a mRNA of the invention is modified to M1ψTP and codons comprising A or U, preferably at the third base, are substituted with codons enriched in guanine (G) or cytosine (C).

In another embodiment, the chemical modifications and optimised codons increase mRNA translation by between about 500-1500 fold, by between about 600-1400 fold, by between about 700-1300 fold, by between about 800-1200 fold or by between about 900-1100 fold, when compared to a mRNA without the chemical modifications and optimised codons. In another embodiment, the chemical modifications and optimised codons increase mRNA translation by at least about 500 fold, by at least about 600 fold, by at least about 700 fold, by at least about 800 fold, by at least about 900 fold or by at least about 1000 fold, when compared to a mRNA without the chemical modifications and optimised codons. In another embodiment, the chemical modifications and optimised codons increase mRNA translation by about 1000 fold when compared to a mRNA without the chemical modifications and optimised codons. Optionally, this is determinable by luciferase reporter assay.

In another embodiment, the mRNA comprises a cap, 5′UTR, coding sequence, a 3′UTR and a poly A tail. Preferably, the cap comprises a cap 1 mRNA structure as set forth in the sequence AG or a sequence having at least 70%, at least 75%, at least 80%, at least 85%, at least 90%, at least 95%, at least 96%, at least 97%, at least 98%, at least 99%, at least 99.5% at least 99.8% or 100% identity to the sequence AG.

In an embodiment, the 5′UTR and 3′UTR comprise sequences that increase mRNA stability and/or polypeptide expression. In an embodiment, the 5′UTR is an elongation factor, preferably elongation factor 1-beta (EEF1B2), more preferably according to the sequence set forth in SEQ ID NO: 1 or a sequence having at least 70%, at least 75%, at least 80%, at least 85%, at least 90%, at least 95%, at least 96%, at least 97%, at least 98%, at least 99%, at least 99.5% at least 99.8% or 100% identity to a sequence set forth as SEQ ID NO: 1. In another embodiment, the 3′UTR is alpha-1 globin, preferably according to the sequence set forth in SEQ ID NO: 3 or a sequence having at least 70%, at least 75%, at least 80%, at least 85%, at least 90%, at least 95%, at least 96%, at least 97%, at least 98%, at least 99%, at least 99.5% at least 99.8% or 100% identity to a sequence set forth as SEQ ID NO: 3.

In an embodiment, the poly A tail comprises between 25 to 200 nucleotides, between 50 to 150 nucleotides, between 80 to 120 nucleotides, preferably about 100 nucleotides. In another embodiment, the poly A tail comprises a sequence having at least 70%, at least 75%, at least 80%, at least 85%, at least 90%, at least 95%, at least 96%, at least 97%, at least 98%, at least 99%, at least 99.5% at least 99.8% or 100% identity to a sequence set forth as SEQ ID NO: 4.

In an embodiment, the mRNA comprises a sequence having at least 70%, at least 75%, at least 80%, at least 85%, at least 90%, at least 95%, at least 96%, at least 97%, at least 98%, at least 99%, at least 99.5% at least 99.8% or 100% identity to a sequence set forth as SEQ ID NO: 5.

In another aspect, there is provided a mammalian cell, preferably a cell of the central nervous system, liver, spleen or lungs, comprising a mRNA of the invention. In an embodiment, the cell is a hepatocyte, reticular cell, lymphocyte, alveolar epithelial cell, microglia, neuron or oligodendrocyte cell. In an embodiment, the cell is obtained from a subject having a lysosomal storage disease, optionally Niemann-Pick Type C disease, preferably associated with a mutation to an intracellular cholesterol trafficker, most preferably NPC intracellular cholesterol transporter 1 (NPC-1). In an embodiment, the mutation to NPC-1 includes a mutation selected from the group consisting of Gly248Val, Met1142Thr, Ile1061Thr and Arg404Trp. In this embodiment, the lysosomes of the cell comprise an increased amount of lipid, optionally including cholesterol such as unesterified cholesterol, sphingomyelin, glycosphingolipid or bis(monoacylglycerol) phosphate.

In another aspect, there is provided a composition comprising a mRNA of the invention.

In another aspect, there is provided a pharmaceutical composition comprising a mRNA of the invention and a pharmaceutically acceptable carrier, diluent or excipient.

In another aspect, there is provided a kit comprising a mRNA of the invention or a pharmaceutical composition of the invention.

In another aspect, there is provided a method of treating or preventing a lysosomal storage disease in a subject in need thereof, comprising administering a therapeutically effective amount of a mRNA of the invention or a pharmaceutical composition a mRNA of the invention to the subject, thereby treating or preventing the lysosomal storage disease in the subject in need thereof.

In another aspect, there is provided use of a therapeutically effective amount of a mRNA of the invention or a pharmaceutical composition of the invention in the preparation of a medicament for treating or preventing a lysosomal storage disease.

In another aspect, there is provided a mRNA of the invention or a pharmaceutical composition of the invention, for use in treating or preventing a lysosomal storage disease.

In an embodiment, the lysosomal storage disease is associated with impaired intracellular lipid trafficking.

In an embodiment, the lipid is cholesterol, sphingomyelin, glycosphingolipid and/or bis(monoacylglycerol) phosphate.

In an embodiment, the lysosomal storage disease is associated with a mutation to an intracellular cholesterol trafficker, preferably selected from NPC intracellular cholesterol transporter 1 (NPC-1) or NPC intracellular cholesterol transporter 2 (NPC-2), most preferably NPC-1. In an embodiment, the mutation to NPC-1 includes a mutation selected from the group consisting of Gly248Val, Met1142Thr, Ile1061Thr and Arg404Trp.

Preferably, the lysosomal storage disease is Niemann-Pick Type C disease.

In an embodiment, the method further comprises identifying a subject having, or at risk of having a lysosomal storage disorder, preferably Niemann-Pick Type C disease.

In an embodiment, the subject exhibits one or more of the following symptoms:
enlarged liver and spleen (hepatosplenomegaly)
difficulty coordinating movement (ataxia)
abnormal eye movements (vertical supranuclear gaze palsy)
poor muscle tone (hypotonia)
severe liver disease
frequent respiratory infections
difficulty with speech
difficulty with swallowing and feeding
loss of cognitive skills
seizures.

In an embodiment, where prevention of a lysosomal storage disease is contemplated, the subject has no clinical symptoms of the lysosomal storage disease.

In an embodiment, the treatment further comprises administering an inhibitor of glucosylceramide synthase, an inducer of heat shock proteins, a cyclic oligosaccharide or hydroxypropyl-beta-cyclodextrin. In this embodiment, administration may be at the same time or a different time to the administration of a mRNA or pharmaceutical composition of the invention.

In an embodiment, the treatment restores intracellular lipid trafficking in the subject, preferably in cells of the central nervous system (e.g., glial cells and oligodendrocytes), liver (e.g., hepatocytes), spleen (e.g., reticular cells and lymphocytes) and/or lungs (e.g., alveolar epithelial cells), most preferably in cells of the central nervous system (e.g., glial cells and oligodendrocytes). In an embodiment, the treatment improves intracellular lipid trafficking in cells of the central nervous system (e.g., glial cells and oligodendrocytes), liver (e.g., hepatocytes), spleen (e.g., reticular cells and lymphocytes) and/or lungs (e.g., alveolar epithelial cells) by at least 10%, at least 20%, at least 30%, at least 40%, at least 45%, at least 50%, at least 55%, at least 60%, at least 65%, at least 70%, at least 75%, at least 80%, at least 85%, at least 90%, at least 95%, or more when compared to a subject having lysosomal storage disease, preferably Niemann-Pick Type C disease and not treated with a mRNA of the invention.

In an embodiment, intracellular lipid trafficking is restored within lysosomes of cells of the central nervous system (e.g., glial cells and oligodendrocytes), liver (e.g., hepatocytes), spleen (e.g., reticular cells and lymphocytes) and/or lungs (e.g., alveolar epithelial cells), most preferably in lysosomes of the central nervous system (e.g., glial cells and oligodendrocytes).

Thus, in another aspect, there is provided a method of restoring lysosomal lipid trafficking in a subject in need thereof, comprising administering a therapeutically effective amount of a mRNA of the invention or a pharmaceutical composition of the invention to the subject, thereby restoring lysosomal lipid trafficking in the subject in need thereof.

In another aspect, there is provided use of a therapeutically effective amount of a mRNA of the invention or a pharmaceutical composition of the invention in the preparation of a medicament for restoring lysosomal lipid trafficking in a subject in need thereof.

In another aspect, there is provided a mRNA of the invention or a pharmaceutical composition of the invention, for use in restoring lysosomal lipid trafficking in a subject in need thereof.

In another embodiment, the treatment reduces lipid accumulation in lysosomes, preferably in lysosomes of cells of the central nervous system (e.g., glial cells and oligodendrocytes), liver (e.g., hepatocytes), spleen (e.g., reticular cells and lymphocytes) and/or lungs (e.g., alveolar epithelial cells), most preferably in cells of the central nervous system (e.g., glial cells and oligodendrocytes). In an embodiment, the treatment reduces lipid accumulation in lysosomes of cells of the central nervous system (e.g., glial cells and oligodendrocytes), liver (e.g., hepatocytes), spleen (e.g., reticular cells and lymphocytes) and/or lungs (e.g., alveolar epithelial cells) by at least 10%, at least 20%, at least 30%, at least 40%, at least 45%, at least 50%, at least 55%, at least 60%, at least 65%, at least 70%, at least 75%, at least 80%, at least 85%, at least 90%, at least 95% or more, when compared to a subject having lysosomal storage disease, preferably Niemann-Pick Type C disease and not treated with a mRNA of the invention.

Thus, in another aspect, there is provided a method of reducing lysosomal lipid accumulation in a subject in need thereof, comprising administering a therapeutically effective amount of a mRNA of the invention or a pharmaceutical composition of the invention to the subject, thereby reducing lysosomal lipid accumulation in the subject in need thereof.

In another aspect, there is provided use of a therapeutically effective amount of a mRNA of the invention or a pharmaceutical composition of the invention in the preparation of a medicament for reducing lysosomal lipid accumulation in a subject in need thereof.

In another aspect, there is provided a mRNA of the invention or a pharmaceutical composition of the invention, for use in reducing lysosomal lipid accumulation in a subject in need thereof.

In an embodiment, the lipid is cholesterol, sphingomyelin, glycosphingolipid and/or bis(monoacylglycerol) phosphate. In an embodiment, the cholesterol is unesterified cholesterol.

In an embodiment, cholesterol esterification is increased in the subject. In another embodiment, total cholesterol levels are reduced.

In another embodiment, lysosome size is decreased in cells of the central nervous system (e.g., glial cells and oligodendrocytes), liver (e.g., hepatocytes), spleen (e.g., reticular cells and lymphocytes) and lungs (e.g., alveolar epithelial cells), most preferably lysosomal size is decreased in cells of the central nervous system (e.g., glial cells and oligodendrocytes). In an embodiment, the treatment reduces lysosome size of cells of the central nervous system (e.g., glial cells and oligodendrocytes), liver (e.g., hepatocytes), spleen (e.g., reticular cells and lymphocytes) and/or lungs (e.g., alveolar epithelial cells) by at least 10%, at least 20%, at least 30%, at least 40%, at least 45%, at least 50%, at least 55%, at least 60%, at least 65%, at least 70%, at least 75%, at least 80%, at least 85%, at least 90%, at least 95% or more, when compared to a subject having lysosomal storage disease, preferably Niemann-Pick Type C disease and not treated with a mRNA of the invention.

Thus, in another aspect, there is provided a method for reducing lysosome size in a subject in need thereof, comprising administering a therapeutically effective amount of a mRNA of the invention or a pharmaceutical composition of the invention to the subject, thereby reducing lysosome size in a subject in need thereof.

In another aspect, there is provided use of a therapeutically effective amount of a mRNA of the invention or a pharmaceutical composition of the invention in the preparation of a medicament for reducing lysosome size in a subject in need thereof.

In another aspect, there is provided a mRNA of the invention or a pharmaceutical composition of the invention, for use in reducing lysosome size in a subject in need thereof.

In an aspect of the invention, there is provided a method for restoring expression of lysosome NPC intracellular cholesterol transporter 1 (NPC-1) in a subject in need thereof, comprising:
(a) identifying or having identified a subject having a mutation to NPC-1 selected from the group consisting of Gly248Val, Met1142Thr, Ile1061 Thr and Arg404Trp; and
(b) administering a therapeutically effective amount of a mRNA of the invention or a pharmaceutical composition of the invention to the subject, thereby restoring NPC-1 expression in the subject in need thereof.

In another aspect, there is provided use of a therapeutically effective amount of a mRNA of the invention or a pharmaceutical composition of the invention in the preparation of a medicament for restoring expression of lysosomal NPC intracellular cholesterol transporter 1 (NPC-1) in a subject in need thereof, wherein the use optionally comprises:
(a) identifying or having identified a subject having a mutation to NPC-1 selected from the group consisting of Gly248Val, Met1142Thr, Ile1061 Thr and Arg404Trp; and
(b) administering a therapeutically effective amount of a mRNA of the invention or a pharmaceutical composition of the invention to the subject, thereby restoring NPC-1 expression in the subject in need thereof.

In another aspect, there is provided a mRNA of the invention or a pharmaceutical composition of the invention, for use in restoring expression of lysosomal NPC intracellular cholesterol transporter 1 (NPC-1) in a subject in need thereof, wherein the use optionally comprises:
(a) identifying or having identified a subject having a mutation to NPC-1 selected from the group consisting of Gly248Val, Met1142Thr, Ile1061Thr and Arg404Trp; and
(b) administering a therapeutically effective amount of a mRNA of the invention or a pharmaceutical composition a mRNA of the invention to the subject, thereby restoring NPC-1 expression in the subject in need thereof.

In an embodiment, the subject has a mutation to one or both loci of NPC-1. In an embodiment, the treatment restores NPC-1 expression in the subject by at least 10%, at least 20%, at least 30%, at least 40%, at least 45%, at least 50%, at least 55%, at least 60%, at least 65%, at least 70%, at least 75%, at least 80%, at least 85%, at least 90%, at least 95%, when compared to a subject having lysosomal storage disease, preferably Niemann-Pick Type C disease and not treated with a mRNA of the invention.

In an embodiment, the mRNA or pharmaceutical composition is suitable, or formulated for delivery across the blood brain barrier of the subject. In another embodiment, the mRNA or pharmaceutical composition is suitable for delivery to the liver, spleen and/or lungs of the subject.

In another embodiment, the mRNA or pharmaceutical composition is formulated in or as a lipid nanoparticle. In this embodiment, the lipid nanoparticle may be formulated for delivery across the blood brain barrier of the subject.

In an embodiment, the mRNA is encapsulated in a LNP. In another embodiment, the mRNA is bound to a LNP. In another embodiment, the mRNA is adsorbed on to a LNP.

In an embodiment, the LNP comprises a PEG-lipid, a structural lipid and/or a neutral lipid. In an embodiment, the LNP comprises a PEG-lipid, a structural lipid and a neutral lipid. In another example, the LNP comprises a PEG-lipid, a structural lipid or a neutral lipid. In one example, the LNP further comprises a cationic lipid.

In an embodiment, the pharmaceutical composition further comprises a polymeric microparticle. In an embodiment, the mRNA is encapsulated in a polymeric microparticle. In an embodiment, the mRNA is bound to a polymeric microparticle. In another embodiment, the mRNA is adsorbed on to a polymeric microparticle.

In an embodiment, the pharmaceutical composition further comprises an oil-in-water emulsion. For example, the mRNA is encapsulated in an oil-in-water emulsion. In another embodiment, the mRNA is bound to an oil-in-water emulsion. In another embodiment, the mRNA is bound to an oil-in-water emulsion. In another embodiment, the mRNA is adsorbed on to an oil-in-water emulsion. In another embodiment, the mRNA is resuspended in an oil-in-water emulsion.

In another aspect, there is provided a process for identifying an mRNA that provides for increased polypeptide expression of an intracellular lipid trafficker comprising:
i) introducing a mRNA of the invention into a cell under conditions sufficient for the expression of the polypeptide encoded by the mRNA within the cell,
ii) determining whether the level of the intracellular lipid trafficker is increased relative to a corresponding cell lacking the mRNA.

In an embodiment, the intracellular lipid trafficker comprises a sequence having at least 70%, at least 75%, at least 80%, at least 85%, at least 90%, at least 95%, at least 96%, at least 97%, at least 98%, at least 99%, at least 99.5% or at least 99.8% to a sequence set forth as SEQ ID NO: 2, 5, 8 or 9. Preferably, the intracellular lipid trafficker comprises a sequence set forth as SEQ ID NO: 2 or 5.

In another aspect, there is provided a method of synthesis of a mRNA encoding an intracellular lipid trafficker comprising expressing a DNA polynucleotide encoding a mRNA of the invention, thereby synthesising a mRNA encoding an intracellular lipid trafficker. Preferably, the method is carried out using a cell free assay. In an embodiment, the method further comprises introducing a chemical modification to the mRNA.

In an embodiment, the DNA polynucleotide is provided in a plasmid.

In another embodiment, the mRNA encoding an intracellular lipid trafficker comprises a sequence having at least 70%, at least 75%, at least 80%, at least 85%, at least 90%, at least 95%, at least 96%, at least 97%, at least 98%, at least 99%, at least 99.5% or at least 99.8% to a sequence set forth as SEQ ID NO: 2, 5, 8 or 9, or 100% identity to SEQ ID NO: 2 or 5.

In an embodiment, the mRNA comprises an optimised codon and a chemical modification.

In an embodiment, the optimised codon comprises codon substitution with guanine (G) and/or cytosine (C), preferably substituting codons with adenine (A) or uracil (U) at the third base with codons enriched in guanine (G) or cytosine (C). In a preferred embodiment, the chemical modification is a nucleoside modification, preferably N1-methylpseudouridine 5′-triphosphate (M1ψTP).

In another aspect, there is provided a mRNA obtained from a method described herein.

In another aspect, there is provided a DNA polynucleotide for in vitro transcription of an mRNA according to the invention, optionally comprised within a plasmid.

Any discussion of documents, acts, materials, devices, articles or the like which has been included in the present specification is not to be taken as an admission that any or all of these matters form part of the prior art base or were common general knowledge in the field relevant to the present disclosure as it existed before the priority date of each of the appended claims.

Any embodiment herein shall be taken to apply mutatis mutandis to any other embodiment unless specifically stated otherwise.

The present invention is not to be limited in scope by the specific embodiments described herein, which are intended for the purpose of exemplification only. Functionally-equivalent products, compositions and methods are clearly within the scope of the invention, as described herein.

Throughout this specification, unless specifically stated otherwise or the context requires otherwise, reference to a single step, composition of matter, group of steps or group of compositions of matter shall be taken to encompass one and a plurality (i.e. one or more) of those steps, compositions of matter, groups of steps or group of compositions of matter.

The invention is hereinafter described by way of the following non-limiting Examples and with reference to the accompanying figures.

### BRIEF DESCRIPTION OF THE ACCOMPANYING DRAWINGS

FIG. 1. Overview of mRNA engineering workflow (A) Codon optimization and chemical base modification are two separate engineering strategies that can be used to enhance mRNA translation. Different codon optimization strategies alter the base content of an mRNA's coding sequence in different ways. GC3 optimization results in a significant overall enrichment in G and C base content. (B) An overview of the mRNA synthesis and purification workflow used throughout this study.

FIG. 2. In vitro expression and thermal stability profiles of wildtype versus codon-optimized Luc mRNAs. (A) Protein expression profiles for Luc mRNA variants harboring all unmodified bases, across HeLa, U87 MG, and Hep G2 cell lines. (B) Protein expression profiles for N1-methylpseudouridine triphosphate (M1ψTP) base-modified Luc mRNA variants, across HeLa, U87 MG, and Hep G2 cell lines. (C, D) Thermal stability curves for unmodified and M1ψTP-modified mRNA variants, as measured by differential scanning fluorimetry. Curves were generated by plotting the negative first derivative of the fluorescence trace as a function of temperature. mRNA melting temperature is denoted by the ▾ icon. (E) RNAfold predictions of optimal secondary structure and minimum free energy for unmodified mRNA variants. Colors indicate base-pairing probabilities, with blue indicating lower probability. In (A) and (B), cells were seeded at a density of 10,000 per well, transfected with 200 ng mRNA, and assayed for protein expression 24 hours after treatment. Data are mean±s.d. of at least three independent experiments and were analyzed by ordinary one-way ANOVA followed by a Dunnett's multiple comparisons test. **P<0.01, ***P<0.001, and ****P<0.0001. In (C), all mRNAs were run in duplicate and for three consecutive heating/cooling cycles. Measurements were taken on three separate occasions. Curves represent the mean of all independent runs, using data taken from the second heating/cooling cycle.

FIG. 3. In vitro expression profiles for codon-optimized Luc mRNAs harboring different base modifications. (A) Protein expression profiles for GC3-optimized and GeneArt-optimized Luc mRNAs harboring either all unmodified bases, M1ψTP-modified bases, 5moUTP-modified bases, or M1ψTP- and 5mCTP-modified bases. Expression was assayed across HeLa, U87 MG, and Hep G2 cell lines. (B) Protein expression profiles for GC3-optimized, M1ψTP-modified Luc mRNA at 100 ng, 10 ng, 1 ng, and 0.1 ng, compared to wildtype, unmodified Luc mRNA at 100 ng. In (A), cells were seeded at a density of 10,000 per well, transfected with 200 ng mRNA, and assayed for protein expression after 24 hours. Data are mean±s.d. of at least three independent experiments. In (B), HeLa and U87 MG cells were seeded at a density of 10,000 per well, while Hep G2 cells were seeded at a density of 15,000 per well. Protein expression was assayed 24 hours post-transfection. Data are mean±s.d. of three technical replicates. Luciferase expression at 6 hours, 24 hours, and 48 hours post-transfection with M1ψTP-modified GC3 Luc mRNA in (C) HeLa cells, (D) U87 MG cells, and (E) Hep G2 cells.

FIG. 4. mRNA treatment rescues NPC1 protein insufficiency and the disease phenotype in patient fibroblasts. (A) Overview of the rationale behind mRNA-based treatment of NPC1 disease. (B) NPC1 and Hoechst 33342 staining in healthy fibroblasts (GM03652), Lipofectamine-treated patient fibroblasts (GM18393), and GM18393 patient fibroblasts treated with 50 ng of M1ψTP-modified NPC1 mRNA. Cells were seeded at a density of 3000 per well and imaged 24 hours after treatment. Images are representative of three independent experiments. (C) Quantification of NPC1 staining from the experiment in (B), as measured by average NPC1 fluorescence per cell (cumulative NPC1 fluorescence was divided by the total number of cells identified via Hoescht staining, using the PerkinElmer Harmony high-content analysis software). Data are mean±s.d. of three technical replicates, encompassing 7 imaging fields per well and >300 cells per treatment condition. (D) Quantification of total and unesterified cholesterol levels in healthy cells, Lipofectamine-treated GM18393 cells, and GM18393 cells treated with 50 ng NPC1 M1ψTP mRNA, after a 48-hour incubation in lipoprotein-deficient serum followed by either a 6-hour incubation with 50 μg/ml LDL or a further 6-hour incubation with lipoprotein-deficient serum to establish a baseline control. Total and unesterified cholesterol levels were assayed using the Amplex Red Cholesterol Assay Kit. Data are mean±s.d. of three technical replicates, representing three independent experiments. (E) Filipin and Lysotracker Red DND-99 staining in healthy cells, Lipofectamine-treated GM18393 cells, and 50 ng mRNA-treated GM18393 cells after a 48-hour incubation in lipoprotein-deficient serum followed by 6-hour incubation with 50 μg/ml LDL. Images are representative of three independent experiments. (F) Quantification of filipin staining from the experiment in (E), as measured by average filipin fluorescence per well. Data are mean±s.d. of three technical replicates, encompassing 9 imaging fields per well and >600 cells per treatment condition. (G) Quantification of lysosome size from Lysotracker Red staining in (E), using the PerkinElmer Harmony high-content analysis software. Data are mean±s.d. of two technical replicates, encompassing 9 imaging fields per well and >300 cells per treatment condition. In (C), (F) and (G), *P<0.05, **P<0.01 by ordinary one-way ANOVA followed by a Dunnett's multiple comparisons test. In (D), ****P<0.0001 by two-way ANOVA followed by a Tukey's multiple comparisons test.

FIG. 5. Transfection with engineered NPC1 mRNA increases NPC1 protein expression in fibroblasts derived from two different NPC1 patients. (A) NPC1 and Hoechst 33342 staining in GM18393 patient fibroblasts treated with Lipofectamine, 50 ng of unmodified NPC1 mRNA, or 50 ng of 5moUTP-modified NPC1 mRNA. Cells were seeded at a density of 3000 per well and imaged 24 hours after treatment. Images are representative of three independent experiments. (B) NPC1 and Hoechst 33342 staining in GM17919 patient fibroblasts treated with Lipofectamine, 50 ng of 5moUTP-modified NPC1 mRNA, 50 ng of M1ψTP-modified NPC1 mRNA, or 50 ng of unmodified NPC1 mRNA. Cells were seeded at a density of 3000 per well and imaged 24 hours after treatment. Images are representative of two independent experiments.

FIG. 6. M1ψTP-modified NPC1 mRNA exerts an inverse phenotypic dose-response effect on GM18393 cells. (A) Quantification of free, unesterified cholesterol levels in GM18393 cells after treatment with Lipofectamine or three descending doses of M1ψTP-modified NPC1 mRNA (100 ng, 50 ng, 25 ng). Surprisingly, only the 25 ng mRNA dose produced a statistically meaningful reduction in free cholesterol levels. Cells were seeded at a density of 3000 per well in full serum media and assayed 48 hours after transfection. Data are mean±s.d. of three technical replicates, representing three independent experiments. **P<0.01 by ordinary one-way ANOVA followed by a Dunnett's multiple comparisons test. (B) Quantification of filipin staining in healthy cells (GM03652), Lipofectamine-treated GM18393 cells, and GM18393 cells treated with three descending doses of M1ψTP-modified NPC1 mRNA (200 ng, 100 ng, 50 ng), after a 48-hour incubation in lipoprotein-deficient serum followed by 6-hour incubation with 50 μg/ml LDL. Data are mean±s.d. of three technical replicates, encompassing 9 imaging fields per well and >600 cells per treatment condition. Images were quantified using the PerkinElmer Harmony high-content analysis software.

### KEY TO THE SEQUENCE LISTING

SEQ ID NO: 1—mRNA sequence of the EEF1B2-001 5′untranslated region (5′UTR).

SEQ ID NO: 2—mRNA sequence of the GC3-optimized NPC1 coding region.

SEQ ID NO: 3—mRNA sequence of the alpha-1 globin 3′ untranslated region (3′UTR).

SEQ ID NO: 4-mRNA sequence of the 100-nt poly(A) tail.

SEQ ID NO: 5-mRNA sequence of the GC3-optimized NPC1 gene comprising a Cap1 structure, 5′UTR, coding region, 3′UTR and poly A tail.

SEQ ID NO: 6-mRNA sequence of the GC3-optimized luciferase coding region.

SEQ ID NO: 7-mRNA sequence of the GC3-optimized luciferase gene comprising a Cap1 structure, 5′UTR, coding region, 3′UTR and poly A tail.

SEQ ID NO: 8-Wild-type mRNA sequence of Homo sapiens NPC intracellular cholesterol transporter 1 (NPC1) (based on DNA defined in NM_000271.5).

SEQ ID NO: 9-Wild-type mRNA sequence of Homo sapiens NPC intracellular cholesterol transporter 2 (NPC2) (based on DNA defined in NM_001363688.1).

SEQ ID NO: 10-Wild-type amino acid sequence of Homo sapiens NPC intracellular cholesterol transporter 1 (NPC1) (NP_000262.2).

SEQ ID NO: 11-Wild-type amino acid sequence of Homo sapiens NPC intracellular cholesterol transporter 2 (NPC2) (NP_001350617.1).

SEQ ID NO: 12-Primer sequence for the generation of DNA template.

SEQ ID NO: 13-Primer sequence for the generation of DNA template.

### DETAILED DESCRIPTION OF THE INVENTION

### General Techniques and Definitions

Unless specifically defined otherwise, all technical and scientific terms used herein shall be taken to have the same meaning as commonly understood by one of ordinary skill in the art (e.g., in cell culture, molecular genetics, plant molecular biology, plant cannabinoid synthesis, protein chemistry, and biochemistry).

Unless otherwise indicated, the recombinant polynucleotide, polypeptide, cell and plant culture, and immunological techniques utilized in the present invention are standard procedures, well known to those skilled in the art. Such techniques are described and explained throughout the literature in sources such as Perbal (1984), Sambrook (1989), Brown (1991), Glover and Hames (1995 and 1996), Ausubel et al. (1988) and Coligan et al. (including all updates until present).

The term “and/or”, e.g., “X and/or Y” shall be understood to mean either “X and Y” or “X or Y” and shall be taken to provide explicit support for both meanings or for either meaning.

Throughout this specification the word “comprise”, or variations such as “comprises” or “comprising”, will be understood to imply the inclusion of a stated element, integer or step, or group of elements, integers or steps, but not the exclusion of any other element, integer or step, or group of elements, integers or steps.

The term “about” and the use of ranges in general, whether or not qualified by the term about, means that the number comprehended is not limited to the exact number set forth herein, and is intended to refer to ranges substantially within the quoted range while not departing from the scope of the invention. As used herein, “about” will be understood by persons of ordinary skill in the art and will vary to some extent on the context in which it is used. If there are uses of the term which are not clear to persons of ordinary skill in the art given the context in which it is used, “about” will mean up to plus or minus 10%, more preferably 5%, more preferably 1%, of the particular term.

mRNA of the Invention

In an aspect of the invention, there is provided a messenger ribonucleic acid (mRNA) that encodes an intracellular lipid trafficker that includes a codon optimisation and/or chemical modification. mRNA is a single-stranded molecule of ribonucleic acid (RNA) that corresponds to the genetic sequence of a gene. mRNA is created during the process of transcription, where an enzyme (RNA polymerase) converts the gene into primary transcript mRNA (also known as pre-mRNA).

In accordance with the invention, the term “messenger RNA” (mRNA) refers to a polynucleotide which encodes a polypeptide of interest and which is capable of being translated to produce an encoded polypeptide in vitro, in vivo, in situ or ex vivo, preferably in vivo. The term “mRNA” may be used interchangeably with “mRNA molecule” herein.

In producing an mRNA of the invention, a skilled person would understand that wild-type mRNA sequences such as those defined in SEQ ID NO: 8 and SEQ ID NO: 9 can be synthesised according to the methods described herein or known in the art including those described in the Example. It is envisaged that the mRNA sequence is synthesised by in vitro transcription from a DNA template and that the DNA sequence encoding the mRNA is codon optimised, preferably enriched for G and C bases, more preferably at the third base of each codon. Preferably, the mRNA sequence is then chemically modified to increase mRNA stability and/or mRNA translation when compared to a mRNA without chemical modification. Preferably, the chemical modification is a nucleoside modification whereby a base is modified to a uracil (U) or a cytosine (C) but the modification may be to any mRNA base including adenine (A), and guanine (G) if the modification increases mRNA stability and/or mRNA translation when compared to a mRNA without chemical modification. It will be understood that by introducing a codon optimisation and/or chemical modification of a mRNA of the invention, this allows for restoration or increased protein expression and functionality that approximates the function of a wild-type protein or exceeds the function of a wild-type protein. A skilled person will understand that protein expression by means of a chemical and/or nucleoside modification, preferably of NPC1, will be enhanced or restored when compared to a corresponding mRNA that does not contain the chemical and/or nucleoside modification.

In an embodiment, the “corresponding mRNA” may be a mRNA without the chemical and/or nucleoside modification which may also be referred to as a wild-type mRNA. A “wildtype mRNA” refers to any mRNA wild-type gene that is capable of having normal (level of function absent disease or disorder) biological activity when expressed as a protein in vivo.

Where it is contemplated that a mRNA of the invention provides for restored intracellular lipid trafficking, this is typically understood to mean that the mRNA has restored the cells' capacity for cholesterol esterification, which is preferably associated with a reduction in free cholesterol levels. Typically, the capacity for cholesterol esterification will be about the same as the capacity of a wild-type NPC1 polypeptide, however in some cases, it may be about 70%, about 75%, about 80%, about 85%, about 90%, about 95%, about 96%, about 97%, about 98%, about 99% or about 99.5% or higher when compared to a wild-type NPC1 polypeptide. The wild-type polypeptide may be found for instance, in a subject not diagnosed as having Niemann-Pick type C disease and not having a mutation in the NPC1 gene.

The term “NPC1” refers to the wildtype NPC1 gene or protein, various mutant forms of which are associated with Niemann-Pick type C disease due to the accumulation of intracellular lipid e.g., unesterified cholesterol. The definition of an NPC1 gene includes the various sequence polymorphisms that exist in the species (e.g., humans) in question.

The term “mutant NPC1 gene,” refers to any non-wildtype NPC1 sequence. Typically, a “mutant NPC1 gene” refers to a non-wildtype sequence that results in an abberant functioning NPC1 protein, and thus, NPC disease. In preferred embodiments, the mutation to NPC-1 includes a mutation selected from the group consisting of Gly248Val, Met1142Thr, Ile1061Thr and Arg404Trp which occur on either both or one loci of the NPC-1 gene. It is understood that mutations to one or both alleles of the NPC1 gene are known to cause Niemann-Pick type C disease. Other types of mutations causing Niemann-Pick type C disease are known in the art.

The term “NPC2” refers to the wildtype NPC2 gene, various mutant forms of which, albeit less frequently, are associated with Niemann-Pick Type C disease by leading to the accumulation of intracellular lipid e.g., unesterified cholesterol. The definition of an NPC2 gene includes the various sequence polymorphisms that exist in the species in question.

The term “mutant NPC2 gene,” refers to any non-wildtype NPC2 sequence. Typically, a “mutant NPC2 gene” refers to a non-wildtype sequence that results in an abberant functioning NPC2 protein, and thus, NPC disease.

In an aspect of the invention, there is provided a mRNA encoding an intracellular lipid trafficker polypeptide. The term “intracellular lipid trafficker” is understood to encompass a polynucleotide or protein, preferably mRNA that is capable of trafficking lipids within a cell, preferably within the lysosomal compartment of a cell. Non-limiting examples of intracellular lipid traffickers include lipid transporters NPC-1 and NPC-2. In an embodiment, the intracellular lipid trafficker contains a codon optimisation and/or chemical modification described herein, preferably both a codon optimisation and a chemical modification.

As relevant to NPC disease, accumulation of intracellular lipid e.g., unesterified cholesterol is known to occur in internal organs throughout the body including the liver, spleens and lungs. Typically, lipid accumulates in lysosomes of hepatocytes of the liver, lysosomes of reticular cells and lymphocytes of the spleen, and/or lysosomes of alveolar epithelial cells of the lungs. However the predominant symptoms of NPC are observed in the central nervous system of the subject where intracellular lipid accumulates within various cell types including neurons, microglia and oligodendrocytes.

In an embodiment, the coding region of the mRNA molecule or the region that encodes a polypeptide such as NPC1 may comprise from about 100 to about 200, from about 200 to about 300, from about 300 to about 400, from about 400 to about 500, from about 500 to about 600, from about 600 to about 700, from about 700 to about 800, from about 800 to about 900, from about 900 to about 1000, from about 1000 to about 1200, from about 1200 to about 1400, from about 1400 to about 1600, from about 1600 to about 1800, from about 1800 to about 2000, from about 2000 to about 2200, from about 2200 to about 2400, from about 2400 to about 2600, from about 2600 to about 2800, from about 2800 to about 3000, from about 3000 to about 3200, from about 3200 to about 3400, from about 3400 to about 3600, from about 3600 to about 3800, from about 3800 to about 4000, from about 4000 to about 4200, from about 4200 to about 4400 or more nucleotides or bases. Preferably, the mRNA molecule is about 3800 or about 500 nucleotides or bases. It is envisaged that a skilled person will be able to modify any NPC1 or NPC2 gene known in the art to generate a mRNA of the invention containing a codon optimisation and a chemical modification. Suitable non-limiting mRNA sequences for use in accordance with the invention include those defined in SEQ ID NOs: 2 and 5.

It is understood that the basic components of an mRNA molecule include at least a 5′ cap, a 5′UTR, a coding region, a 3′UTR, and a poly-A tail (in 5′ to 3′ direction). A non-limiting example of an mRNA molecule of the invention includes the sequence set forth in SEQ ID NO: 5 which comprises the mRNA nucleotide sequence of a GC3-optimized NPC1 gene with a Cap1 structure, 5′UTR, coding region, 3′UTR and poly A tail. The sequence defined in SEQ ID NO: 5 has been codon optimised to provide for enrichment of guanine (G) and cytosine (C) bases. A skilled person will understand that similar optimisation can be made to other mRNA sequences or molecules for the purpose of increasing or restoring protein expression, enhancing mRNA stability, thermal stability or functionality. Such methods may include those described herein including in the Example, or known in the art.

### Untranslated Regions

5′UTRs are regions of a gene that are transcribed but not translated. In mRNA, the 5′UTR starts at the transcription start site and continues to the start codon but does not include the start codon; whereas, the 3′UTR starts immediately following the stop codon and continues until the transcriptional termination signal. 5′UTRs play a role in stability and translation initiation. They harbor signatures like Kozak sequences which are commonly known to be involved in the process by which the ribosome initiates translation of many genes. Kozak sequences have the consensus CCR(NG)CCAUGG, where Risa purine (adenine or guanine) three bases upstream of the start codon (AUG), which is followed by another ‘G’. 5′UTR also have been known to form secondary structures which are involved in elongation factor binding.

By engineering the features typically found in abundantly expressed genes of specific target organs, one can enhance the stability and protein production of mRNA of the invention. For example, introduction of 5′ UTR of liver-expressed mRNA, such as albumin, serum amyloid A, Apolipoprotein NB/E, transferrin, alpha fetoprotein, erythropoietin, or Factor VIII, could be used to enhance expression of a nucleic acid molecule, such as a mRNA, in hepatocytes or other liver cells. Likewise, use of 5′ UTR from other tissue-specific mRNA to improve expression in that tissue is possible for muscle (MyoD, Myosin, Myoglobin, Myogenin, Herculin), for endothelial cells (Tie-1, CD36), for myeloid cells (C/EBP, AML1, G-CSF, GM-CSF, CD11b, MSR, Fr-1, i-NOS), for leukocytes (CD45, CD18), for adipose tissue (CD36, GLUT4, ACRP30, adiponectin) and for lung epithelial cells (SP-NB/C/D). Other suitable 5′UTRs are known in the art. A suitable non-limiting example of a 5′UTR that is useful in the invention is the sequence set forth in SEQ ID NO: 1. Other 5′UTRs are well known in the art.

Other non-UTR sequences may be incorporated into the 5′ (or 3′ UTR) UTRs. For example, intrans or portions of intrans sequences may be incorporated into the flanking regions of the polynucleotides, primary constructs or mmRNA of the invention. Incorporation of intronic sequences may increase protein production as well as mRNA levels.

3′ UTRs are known to have stretches of menosines and uridines embedded in them. These AU rich signatures are particularly prevalent in genes with high rates of turnover. Based on their sequence features and functional properties, the AU rich elements (AREs) can be separated into three classes: Class I AREs contain several dispersed copies of an AUUUA motif within U-rich regions. C-Myc and MyoD contain class I AREs. Class II AREs possess two or more overlapping UUAUUUA (U/A) (U/A) nonamers. Molecules containing this type of AREs include GM-CSF and TNF-a. Class III ARES are less well defined. These U rich regions do not contain an AUUUA motif. c-Jun and Myogenin are two well-studied examples of this class. Most proteins binding to the AREs are known to destabilize the messenger, whereas members of the ELAV family, most notably HuR, have been documented to increase the stability of mRNA. HuR binds to AREs of all the three classes. Therefore, it is invisagesd that HuR specific binding sites may be engineered into the 3′ UTR of nucleic acid molecules which will lead to HuR binding and thus, stabilization of the message in vivo.

Introduction, removal or modification of 3′ UTR AU rich elements (AREs) can be used to modulate the stability of mRNA of the invention. Although less preferable, when engineering specific mRNA, one or more copies of an ARE can be introduced to make mRNA of the invention less stable and thereby curtail translation and decrease production of the resultant protein. Likewise, AREs can be identified and removed or mutated to increase the intracellular stability and thus increase translation and production of the resultant protein. Transfection experiments can be conducted in relevant cell lines, using mRNA of the invention and protein production can be assayed at various time points post-transfection. For example, cells can be transfected with different ARE-engineering molecules and by using an ELISA kit to the relevant protein and assaying protein produced at 6 hour, 12 hour, 24 hour, 48 hour, and 7 days post-transfection.

A sutable non-limiting example of a 3′UTR that is useful in the invention is the sequence set forth in SEQ ID NO: 3. Other 3′UTRs are well known in the art.

### 5′ Capping

The 5′ cap structure of an mRNA is involved in nuclear export, increasing mRNA stability and binds the mRNA Cap Binding Protein (CBP), which is responsibile for mRNA stability in the cell and translation competency through the association of CBP with poly(A) binding protein to form the mature cyclic mRNA species. The cap further assists the removal of 5′ proximal intrans removal during mRNA splicing.

Endogenous mRNA molecules may be 5′-end capped generating a 5′-ppp-5′-triphosphate linkage between a terminal guanosine cap residue and the 5′-terminal transcribed sense nucleotide of the mRNA molecule. This 5′-guanylate cap may then be methylated to generate an NY-methyl-guanylate residue. The ribose sugars of the terminal and/or anteterminal transcribed nucleotides of the 5′ end of the mRNA may optionally also be 2′-O-methylated. 5′-decapping through hydrolysis and cleavage of the guanylate cap structure may target a nucleic acid molecule, such as an mRNA molecule, for degradation.

Modifications to the mRNA of the present invention may generate a non-hydrolyzable cap structure preventing decapping and thus increasing mRNA half-life. Because cap structure hydrolysis requires cleavage of 5′-ppp-5′ phosphorodiester linkages, modified nucleotides may be used during the capping reaction. For example, a Vaccinia Capping Enzyme from New England Biolabs (Ipswich, MA) may be used with a-thio-guanosine nucleotides according to the manufacturer's instructions to create a phosphorothioate linkage in the 5′-ppp-5′ cap. Additional modified guanosine nucleotides may be used such as a-methyl-phosphonate and seleno-phosphate nucleotides.

Additional modifications include, but are not limited to, 2′-O-methylation of the ribose sugars of 5′-terminal and/or 5′-anteterminal nucleotides of the mRNA on the 2′-hydroxyl group of the sugar ring. Multiple distinct 5′-cap structures can be used to generate the 5′-cap of a nucleic acid molecule, such as an mRNA molecule.

Cap analogs, or synthetic cap analogs, chemical caps, chemical cap analogs, or structural or functional cap analogs, differ from natural (i.e. endogenous, wild-type or physiological) 5′-caps in their chemical structure, while retaining cap function. Cap analogs may be chemically (i.e. non-enzymatically) or enzymatically synthesized and/or linked to a nucleic acid molecule. For example, the Anti-Reverse Cap Analog (ARCA) cap contains two guanines linked by a 5′-5′-triphosphate group, wherein one guanine contains an N7 methyl group as well as a 3′-O-methyl group (i.e., N7,3′-O-dimethyl-guanosine-5′-triphosphate-5′-guanosine (m7G-3′mppp-G; which may equivaliently be designated 3′ O-Me-m7G (5′)ppp(5′) G). The 3′-0 atom of the other, unmodified, guanine becomes linked to the 5′-terminal nucleotide of the capped nucleic acid molecule (e.g. an mRNA or mmRNA). The N7- and 3′-O-methlyated guanine provides the terminal moiety of the capped nucleic acid molecule (e.g. mRNA or mmRNA). Another cap is mCAP, which is similar to ARCA but has a 2′-O-methyl group on guanosine (i.e., N7,2′-O-dimethyl-guanosine-5′triphosphate-5′-guanosine, m⁷Gm-ppp-G).

While cap analogs allow for the concomitant capping of a nucleic acid molecule in an in vitro transcription reaction, up to 20% of transcripts can remain uncapped. This, as well as the structural differences of a cap analog from an endogenous 5′-cap structures of nucleic acids produced by the endogenous, cellular transcription machinery, may lead to reduced translational competency and reduced cellular stability.

mRNA of the invention may also be capped post-transcriptionally, using enzymes, in order to generate a cap representative of an endogenous, wild-type, natural or physiological cellular function and/or structure as compared to synthetic features or analogs, etc., of the prior art, or which outperforms the corresponding endogenous, wild-type, natural or physiological feature in one or more respects. Non-limiting examples of more authentic 5′-cap structures are those which, among other things, have enhanced binding of cap binding proteins, increased half life, reduced susceptibility to 5′ endonucleases and/or reduced 5′decapping, as compared to synthetic 5′cap structures known in the art (or to a wild-type, natural or physiological 5′cap structure). For example, recombinant Vaccinia Virus Capping Enzyme and recombinant 2′-O-methyltransferase enzyme can create a canonical 5′-5′triphosphate linkage between the 5′-terminal nucleotide of an mRNA and a guanine cap nucleotide wherein the cap guanine contains an N7 methylation and the 5′-terminal nucleotide of the mRNA contains a 2′-O-methyl. Such a structure is termed the Cap1 structure. This cap results in a higher translational-competency and cellular stability and a reduced activation of cellular pro-infiammatory cytokines, as compared, e.g., to other 5′cap analog structures known in the art. Cap structures include, but are not limited to 7mG(5′)ppp(5′)N,pN2p (cap 0), 7mG(5′)ppp(5′)N1mpNp (cap 1), and 7mG(5′)ppp(5′) N1mpN2mp (cap 2).

Because the mRNA may be capped post-transcriptionally, and because this process is more efficient, it is preferred that the mRNA be capped.

According to the present invention, 5′ terminal caps may include endogenous caps or cap analogs. According to the present invention, a 5′ terminal cap may comprise a guanine analog. Useful guanine analogs include, but are not limited to, inosine, N1-methyl-guanosine, 2′fluoro-guanosine, 7-deaza-guanosine, 8-oxo-guanosine, 2-amino-guanosine, LNA-guanosine, and 2-azido-guanosine.

A sutable non-limiting example of a cap structure that is useful in the invention is a Cap 1 structure, preferably the sequence as set forth in the sequence, AG. A skilled person will be able to determine other suitable cap structures useful in the invention.

### IRES Sequences

Further, provided are mRNA which may contain an internal ribosome entry site (IRES). IRES plays an important role in initiating protein synthesis in absence of the 5′ cap structure. An IRES may act as the sole ribosome binding site, or may serve as one of multiple ribosome binding sites of an mRNA. mRNA containing more than one functional ribosome binding site may encode several peptides or polypeptides that are translated independently by the ribosomes (“multicistronic nucleic acid molecules”). When mRNA are provided with an IRES, further optionally provided is a second translatable region. Examples of IRES sequences that can be used according to the invention include without limitation, those from picornaviruses (e.g. FMDV), pest viruses (CFFV), polio viruses (PV), encephalomyocarditis viruses (ECMV), foot-and-mouth disease viruses (FMDV), hepatitis C viruses (HCV), classical swine fever viruses (CSFV), murine leukemia virus (MLV), simian immune deficiency viruses (SIV) or cricket paralysis viruses (CrPV).

### Poly-A Tails

During RNA processing, a long chain of adenine nucleotides (poly-A tail) may be added to a polynucleotide such as an mRNA molecule in order to increase stability. Immediately after transcription, the 3′ end of the transcript may be cleaved to free a 3′ hydroxyl. Then poly-A polymerase adds a chain of adenine nucleotides to the RNA. The process, called polyadenylation, adds a poly-A tail that can be between, for example, approximately 100 and 250 residues long.

Generally, the length of a poly-A tail of the present invention is greater than 30 nucleotides in length. In another embodiment, the poly-A tail is greater than 35 nucleotides in length (e.g., at least or greater than about 35, 40, 45, 50, 55, 60, 70, 80, 90, 100, 120, 140, 160, 180, 200, 250, 300, 350, 400, 450, 500, 600, 700, 800, 900, 1,000, 1,100, 1,200, 1,300, 1,400, 1,500, 1,600, 1,700, 1,800, 1,900, 2,000, 2,500, and 3,000 nucleotides). In some embodiments, the polynucleotide or mRNA includes a poly-A tail from about 30 to about 3,000 nucleotides (e.g., from 30 to 50, from 30 to 100, from 30 to 250, from 30 to 500, from 30 to 750, from 30 to 1,000, from 30 to 1,500, from 30 to 2,000, from 30 to 2,500, from 50 to 100, from 50 to 250, from 50 to 500, from 50 to 750, from 50 to 1,000, from 50 to 1,500, from 50 to 2,000, from 50 to 2,500, from 50 to 3,000, from 100 to 500, from 100 to 750, from 100 to 1,000, from 100 to 1,500, from 100 to 2,000, from 100 to 2,500, from 100 to 3,000, from 500 to 750, from 500 to 1,000, from 500 to 1,500, from 500 to 2,000, from 500 to 2,500, from 500 to 3,000, from 1,000 to 1,500, from 1,000 to 2,000, from 1,000 to 2,500, from 1,000 to 3,000, from 1,500 to 2,000, from 1,500 to 2,500, from 1,500 to 3,000, from 2,000 to 3,000, from 2,000 to 2,500, and from 2,500 to 3,000).

In another embodiment, the poly A tail comprises between 25 to 200 nucleotides, between 50 to 150 nucleotides, between 80 to 120 nucleotides, preferably about 100 nucleotides. In another embodiment, the poly A tail comprises a sequence having at least 70%, at least 75%, at least 80%, at least 85%, at least 90%, at least 95%, at least 96%, at least 97%, at least 98%, at least 99%, at least 99.5% at least 99.8% or 100% identity to a sequence set forth as SEQ ID NO: 4.

In one embodiment, the poly-A tail is designed relative to the length of the overall mmRNA. This design may be based on the length of the coding region, the length of a particular feature or region (such as the first or flanking regions), or based on the length of the ultimate product expressed from the mRNA.

In this context the poly-A tail may be 10, 20, 30, 40, 50, 60, 70, 80, 90, or 100% greater in length than the polynucleotides or mRNA or feature thereof. The poly-A tail may also be designed as a fraction of mRNA to which it belongs. In this context, the poly-A tail may be 10, 20, 30, 40, 50, 60, 70, 80, or 90% or more of the total length of the construct or the total length of the construct minus the poly-A tail. Further, engineered binding sites and conjugation of mRNA for Poly-A binding protein may enhance expression.

Additionally, multiple distinct mRNA may be linked together to the PABP (Poly-A binding protein) through the 3′-end using modified nucleotides at the 3′-terminus of the poly-A tail. Transfection experiments can be conducted in relevant cell lines at and protein production can be assayed by ELISA at 12 hr, 24 hr, 48 hr, 72 hr and day 7 post-transfection.

In one embodiment, a mRNA of the present invention may be designed to include a polyA-G Quartet. The G-quartet is a cyclic hydrogen bonded array of four guanine nucleotides that can be formed by G-rich sequences in both DNA and RNA. In this embodiment, the Gquartet is incorporated at the end of the poly-A tail. The resultant mRNA construct is assayed for stability, protein production and other parameters including half-life at various time points. It has been discovered that the polyA-G quartet results in protein production equivalent to at least 75% of that seen using a poly-A tail of 120 nucleotides alone.

### Codon Optimisation

The nucleotide sequence of a mRNA of the invention is preferably codon optimized. In performing codon optimisation, codon frequencies in target and host organisms are generally matched to ensure proper folding, bias GC content to increase mRNA stability or reduce secondary tructures, minimize tandem repeat codons or base runs that may impair gene construction or expression, customize transcriptional and translational control regions, insert or remove protein trafficking sequences, remove/add post translation modification sites in encoded protein (e.g., glycosylation sites), add, remove or shuffle protein domains, insert or delete restriction sites, modify ribosome binding sites and mRNA degradation sites, to adjust translational rates to allow the various domains of the protein to fold properly, or to reduce or eliminate problem secondary structures within the mRNA.

Codon composition is known to affect translation efficiency. Replacing rare codons with synonymous frequent codons improves translational yield because reuse of the same tRNA accelerates translation owing to amino-acylation of tRNAs in the vicinity of the ribosomes. Codon context (that is, neighbouring nucleotides and codons) also affects the translational elongation rate and translational efficiency. Similar to recombinant DNA-based approaches, codon-optimized in vitro transcribed (IVT) mRNAs have been successfully used. However, in some cases, there may be valid reasons to refrain from using optimized codons as understood by a skilled person. Some proteins require slow translation, which is ensured by rare codons, for their proper folding. It may also be beneficial for some IVT mRNA-encoded to maintain the original ORF.

In a preferred embodiment of the invention, codon optimization methods are useful to increase expression, structural stability, thermal stability or increased function of the encoded protein. Codon optimization tools, algorithms and services are known in the art, and non-limiting examples include services from GeneArt (Life Technologies), DNA2.0 (Menlo Park CA) or other methods known in the art. However, specific strategies for codon optimization vary considerably based on underlying assumptions about which codon features are important to translation. One approach involves substituting in the most frequently used codon for all instances of a given amino acid. Another approach involves only replacing rare codons with more abundant synonymous codons. Still other approaches involve adjusting the codon usage frequency to match the natural frequencies in a host organism, or choosing codons based on cognate transfer RNA (tRNA) abundance. A skilled person will generally understand that codon optimization involves the replacement of a codon with an optimized codon that is synonymous with the replaced codon.

In one embodiment, the ORF sequence is optimized using optimization algorithms. In a preferred embodiment, codon optimisation is conducted by enriching a DNA template that will encode a mRNA of the invention for guanine (G) and cytosine (C) content. Even more preferably, G and C content is enriched by substituting codons with adenine (A) or uracil (U) at the third base with codons enriched in guanine (G) or cytosine (C). A skilled person will understand methods for determing suitable nucleotide substitutions based on codon options for each amino acid as outlined in Table 2 herein and known in the art. Non-limiting examples of sequences that may be codon optimised in accordance with the invention include SEQ ID NO: 2. A skilled person will understand that SEQ ID NO: 9, being a wild-type mRNA sequence of NPC2, may be codon optimised in a similar manner.

In an embodiment of the invention, an codon optimised NPC-1 sequence in accorcance with the invention may comprise a sequence having at least 30%, at least 40%, at least 50%, at least 60%, at least 70%, at least 75%, at least 80%, at least 85%, at least 90%, at least 95%, at least 96%, at least 97%, at least 98%, at least 99%, at least 99.5% at least 99.8% or 100% identity to a sequence set forth as SEQ ID NO: 2 or 8.

Whilst preferable, codon optimised mRNA need not be uniformly cocon optimised along the entire length of the mRNA molecule. Different nucleotide modifications and/or backbone structures may exist at various positions in the nucleic acid. One of ordinary skill in the art will appreciate that the modification(s) may be located at any position(s) of a mRNA such that the polypeptide function, polypeptide expression, mRNA thermal stability or structure is preferably increased or improved. A modification may also be a 5′ or 3′ teminal modification. Further, the mRNA may contain at a minimum one and at maximum 100% optimised codons, or any intervening percentage, such as at least 10%, at least 20%, at least 30%, at least 40%, at least 50%, at least 60%, at least 70%, at least 80%, or at least 90% optimised codons.

In another embodiment of the invention, a codon optimised NPC-2 sequence in accordance with the invention may comprise a sequence having at least 30%, at least 40%, at least 50%, at least 60%, at least 70%, at least 75%, at least 80%, at least 85%, at least 90%, at least 95%, at least 96%, at least 97%, at least 98%, at least 99%, at least 99.5% at least 99.8% or 100% identity to a sequence set forth as SEQ ID NO: 9.

The % identity of a polynucleotide or mRNA is determined by GAP (Needleman and Wunsch, 1970) analysis (GCG program) with a gap creation penalty=5, and a gap extension penalty=0.3. The query sequence is at least 900 nucleotides in length, and the GAP analysis aligns the two sequences over a region of at least 900 nucleotides. Preferably, the query sequence is at least 975 nucleotides in length, and the GAP analysis aligns the two sequences over a region of at least 975 nucleotides. Even more preferably, the query sequence is at least 1,050 nucleotides in length and the GAP analysis aligns the two sequences over a region of at least 1,050 nucleotides. Even more preferably, the GAP analysis aligns two sequences over their entire length.

With regard to the defined polynucleotides or mRNA, it will be appreciated that % identity figures higher than those provided above will encompass preferred embodiments. Thus, where applicable, in light of the minimum % identity figures, it is preferred that the mRNA comprises a sequence which is at least 50%, at least 60%, more preferably at least 70%, more preferably at least 75%, more preferably at least 80%, more preferably at least 85%, more preferably at least 90%, more preferably at least 91%, more preferably at least 92%, more preferably at least 93%, more preferably at least 94%, more preferably at least 95%, more preferably at least 96%, more preferably at least 97%, more preferably at least 98%, more preferably at least 99%, more preferably at least 99.1%, more preferably at least 99.2%, more preferably at least 99.3%, more preferably at least 99.4%, more preferably at least 99.5%, more preferably at least 99.6%, more preferably at least 99.7%, more preferably at least 99.8%, and even more preferably at least 99.9% identical to the relevant nominated SEQ ID NO.

When referring to polynucleotide or mRNA identity herein, it will be understood that a given sequence identity is in reference to the open reading frame sequence.

In a further embodiment, the present invention relates to polynucleotides or mRNA which are substantially identical or identical to those specifically described herein. As used herein, with reference to a mRNA or polynucleotide the term “substantially identical” means the substitution of one or a few (for example 2, 3, or 4) nucleotides whilst maintaining activity of the native protein encoded by the polynucleotide. In addition, this term includes the addition or deletion of nucleotides which results in the increase or decrease in size of the encoded native protein by one or a few (for example 2, 3, or 4) amino acids whilst maintaining activity of the native protein encoded by the polynucleotide.

### Chemical Modifications

The terms “modification” or “modified” refer to modification of a mRNA of the invention with respect to A, G, U or C ribonucleotides. Generally, the modification refers to the coding region of the mRNA. However, the modification may also be introduced into the flanking regions and/or the terminal regions if the modification increases protein expression, function, thermal stability or structure.

Because chemical bonds will necessarily be broken and reformed to effect a structural modification, structural modifications are of a chemical nature and hence are chemical modifications. However, structural modifications will result in a different sequence of nucleotides. For example, the polynucleotide “ATCG” may be chemically modified to “AT-5meC-G”. The same polynucleotide may be structurally modified from “ATCG” to “ATCCCG”. Here, the dinucleotide “CC” has been inserted, resulting in a structural modification to the polynucleotide.

The mRNA or polynucleotides can include any useful modification, such as to the sugar, the nucleobase, or the intemucleoside linkage (e.g. to a linking phosphate/to a phosphodiester linkage to the phosphodiester backbone). For example, the major groove of a polynucleotide, or the major groove face of a nucleobase may comprise one or more modifications. One or more atoms of a pyrimidine nucleohase (e.g. on the major groove face) may he replaced or substituted with optionally substituted amino, optionally substituted thiol, optionally substituted alkyl (e.g., methyl or ethyl), or halo (e.g., chloro or fluoro). In certain cases (e.g., one or more modifications) are present in each of the sugar and the intemucleoside linkage. Modifications according to the present invention may be modifications of ribonucleic acids (RNAs) to deoxyribonucleic acids (DNAs), e.g., the substitution of the 2′OH of the ribofuranysyl ring to 2′H, threose nucleic acids (TNAs), glycol nucleic acids (GNAs), peptide nucleic acids (PNAs), locked nucleic acids (LNAs) or hybrids thereof). Additional modifications are known in the art.

In some embodiments the the chemical modification increases mRNA stability and/or mRNA translation when compared to a mRNA without chemical modification. Preferably the modification is to a uracil (U) or a cytosine (C) but may be to any mRNA base including adenine (A), and guanine (G) if the modification increases mRNA stability and/or mRNA translation when compared to a mRNA without chemical modification.

Suitable uracil modifications may include but are not limited to pseudouridine (ψ), pyridin-4-one ribonucleoside, 5-aza-uridine, 6-aza-uridine, 2-thio-5-aza-uridine, 2-thio-uridine (s2U), 4-thio-uridine (s4U), 4-thiopseudouridine, 2-thio-pseudouridine, 5-hydroxy-uridine (ho5U), 5-aminoallyl-uridine, 5-halo-uridine (e.g., 5-iodo-uridine or 5-bromo-uridine), 3-methyluridine (m3U), 5-methoxy-uridine (mo5U), 5-methoxy-uridine triphosphate (5moUTP), uridine 5-oxyacetic acid (cmo5U), uridine 5-oxyacetic acid methyl ester (mcmo5U), 5-carboxymethyl-uridine (cm5U), 1-carboxymethyl-pseudouridine, 5-carboxyhydroxymethyl-uridine (chm5U), 5-carboxyhydroxymethyl-uridine methyl ester (mchm5U), 5-methoxycarbonylmethyl-uridine (mcm5U), 5-methoxycarbonylmethyl-2-thio-uridine (mcm5s2U), 5-aminomethyl-2-thio-uridine (nm5s2U), 5-methylaminomethyl-uridine (mnm5U), 5-methylaminomethyl-2-thio-uridine (mnm5s2U), 5-methylaminomethyl-2-seleno-uridine (mnm5se2U), 5-carbamoylmethyl-uridine (ncm5U), 5-carboxymethylaminomethyl-uridine (cmnm5U), 5-carboxymethylaminomethyl-2-thio-uridine (cmnm5s2U), 5-propynyluridine, 1-propynyl-pseudouridine, 5-taurinomethyl-uridine, 1-(tm5U), 1-taurinomethyl-pseudouridine, 5-taurinomethyl-2-thio-uridine (tm5s2U), taurinomethyl-4-thio-pseudouridine, 5-methyl-uridine (m5U, i.e., having the nucleobase deoxythymine), 1-methylpseudouridine (m1ψ), 5-methyl-2-thiouridine (m5s2U), 1-methyl-4-thio-pseudouridine (m1s4ψ), 4-thio-1-methyl-pseudouridine, 3-methyl-pseudouridine (m3ψ), 2-thio-1-methyl-pseudouridine, 1-methyl-1-deaza-pseudouridine, 2-thio-1-methyl-1-deaza-pseudouridine, dihydrouridine (D), dihydropseudouridine, 5,6-dihydrouridine, 5-methyldihydrouridine (m5D), 2-thio-dihydrouridine, 2-thio-dihydropseudouridine, 2-methoxy-uridine, 2-methoxy-4-thio-uridine, 4-methoxy-pseudouridine, 4-methoxy-2-thio-pseudouridine, N1-methyl-pseudouridine (also known as 1-methylpseudouridine (m1ψ), 3-(3-amino-3-carboxypropyl) uridine (acp3U), 1-methyl-3-(3-amino-3-carboxypropyl) pseudouridine (acp3ψ), 5-(isopentenylaminomethyl) uridine (inm5U), 5-(isopentenylaminomethyl)-2-thio-uridine (inm5s2U), a-thio-uridine, 2′-O-methyl-uridine (Um), 5,2′-O-dimethyl-uridine (m5Um), 2′-O-methyl-pseudouridine (ψm), 2-thio-2′-O-methyl-uridine (s2Um), 5-methoxycarbonylmethyl-2′-O-methyl-uridine (mcm5Um), 5-carbamoylmethyl-2′-O-methyl-uridine (ncm5Um), 5-carboxymethylaminomethyl-2′-O-methyluridine (cmnm5Um), 3,2′-O-dimethyl-uridine (m3Um), 5-(isopentenylaminomethyl)-2′-O-methyl-uridine (inm5Um), 1-thio-uridine, deoxythymidine, 2′-F-arauridine, 2′-F-uridine, 2′-OH-ara-uridine, 5-(2-carbomethoxyvinyl) uridine, and 5-[3-(1-E-propenylamino) uridine.

Suitable cytosine modifications may include but are not limited to 5-aza-cytidine, 6-aza-cytidine, pseudoisocytidine, 3-methyl-cytidine (m³C), N4-acetyl-cytidine (ac⁴C), 5-formyl-cytidine (f⁵C), N4-methyl-cytidine (m⁴C), 5-methyl-cytidine (m⁵C), 5-methy-lcytidine 5′-triphosphate (5mCTP), 5-halo-cytidine (e.g., 5-iodo-cytidine), 5-hydroxymethyl-cytidine (hm⁵C), 1-methyl-pseudoisocytidine, pyrrolo-cytidine, pyrrolo-pseudoisocytidine, 2-thio-cytidine (s²C), 2-thio-5-methyl-cytidine, 4-thio-pseudoisocytidine, 4-thio-1-methyl-pseudoisocytidine, 4-thio-1-methyl-1-deaza-pseudoisocytidine, 1-methyl-1-deaza-pseudoisocytidine, zebularine, 5-aza-zebularine, 5-methyl-zebularine, 5-aza-2-thio-zebularine, 2-thiozebularine, 2-methoxy-cytidine, 2-methoxy-5-methyl-cytidine, 4-methoxy-pseudoisocytidine, 4-methoxy-1-methyl-pseudoisocytidine, lys idine (k₂C), a-thiocytidine, 2′-O-methyl-cytidine (Cm), 5,2′-O-dimethyl-cytidine (m⁵Cm), N4-acetyl-2′-O-methyl-cytidine (ac⁴Cm), N4,2′-O-dimethyl-cytidine (m⁴Cm), 5-formyl-2′-O-methyl-cytidine (f⁵Cm), N4,N4,2′-O-trimethyl-cytidine (m⁴₂Cm), 1-thio-cytidine, 2′-F-ara-cytidine, 2′-F-cytidine, and 2′-OH-ara-cytidine.

Modified nucleic acids need not be uniformly chemically modified along the entire length of the molecule. Different nucleotide modifications and/or backbone structures may exist at various positions in the nucleic acid. One of ordinary skill in the art will appreciate that the modification(s) may be located at any position(s) of a mRNA such that the polypeptide function, polypeptide expression, mRNA thermal stability or structure is preferably increased or improved. A modification may also be a 5′ or 3′ teminal modification. Further, the mRNA may contain at a minimum one and at maximum 100% chemical modifications, preferably nucleoside modifications, or any intervening percentage, such as at least 10%, at least 20%, at least 30%, at least 40%, at least 50%, at least 60%, at least 70%, at least 80%, or at least 90% modified nucleotides.

Methods of Synthesis of mRNA

mRNA for use in accordance with the invention may be prepared according to any available technique including, but not limited to chemical synthesis, enzymatic synthesis, which is generally termed in vitro transcription (IVT), enzymatic or chemical cleavage of a longer precursor, etc. Methods of synthesizing RNAs are known in the art (see, e.g., Gait, M. J., 1984; and Herdewijn, 2005; both of which are incorporated herein by reference).

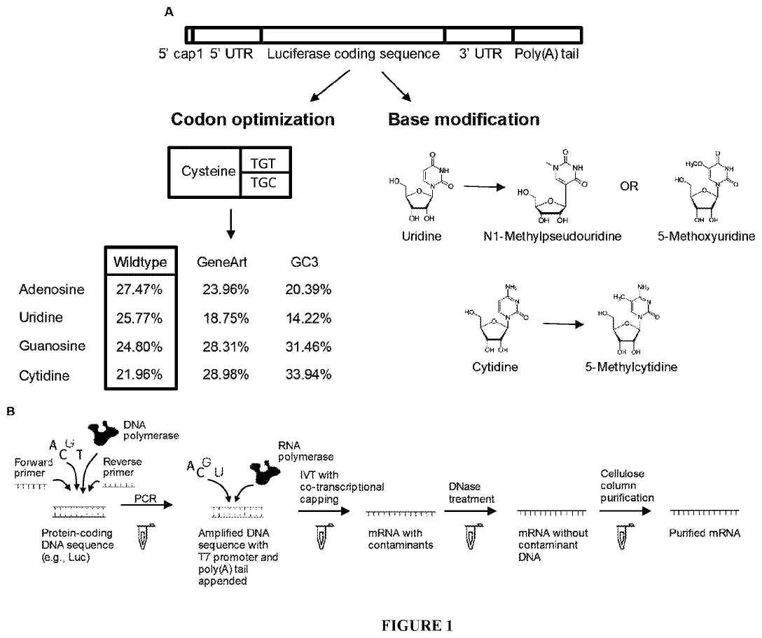

In a preferred embodiment, a mRNA of the invention is prepared by IVT using methods described in the Example (FIG. 1) or known in the art. The machinery of the transfected cell is utilized for in vivo translation of the message to the corresponding protein, which is the therapeutic polypeptide suitable for treating NPC. IVT mRNA is engineered to structurally resemble naturally occurring mature and processed mRNA in the cytoplasm of eukaryotic cells. Hence, the IVT mRNA is single-stranded, has a 5′ cap and a 3′ poly(A) tail. The open reading frame (ORF) encoding the protein of interest is marked by start and stop codons and is flanked by untranslated region (UTRs). The mRNA is generally synthesized in a cell-free system by IVT from a DNA template, such as a linearized plasmid or a PCR product. With the exception of the 5′ cap, this DNA template encodes all the structural elements of a functional mRNA. IVT is performed with T7 or SP6 RNA polymerase in the presence of nucleotides and thereafter the mRNA is capped enzymatically. The template DNA is then digested by DNases and the mRNA is purified by conventionally used methods for isolating nucleic acids.

### Polynucleotides and Genes

The present invention relates to various polynucleotides encoding mRNA, particularly those used in IVT for the generation of a mRNA of the invention. As used herein, a “polynucleotide” or “nucleic acid” or “nucleic acid molecule” means a polymer of nucleotides, which may be DNA or RNA or a combination thereof, and includes genomic DNA, mRNA, cRNA, and cDNA. A given polynucleotide may be of cellular, genomic or synthetic origin, for example made on an automated synthesizer, and may be combined with carbohydrate, lipids, protein or other materials, labelled with fluorescent or other groups, or attached to a solid support to perform a particular activity defined herein, or comprise one or more modified nucleotides not found in nature, well known to those skilled in the art. The polymer may be single-stranded, essentially double-stranded or partly double-stranded. Basepairing as used herein refers to standard basepairing between nucleotides, including G: U basepairs. “Complementary” means two polynucleotides are capable of basepairing (hybridizing) along part of their lengths, or along the full length of one or both. The term “polynucleotide” is used interchangeably herein with the term “nucleic acid”.

The polynucleotides or nucleic acid sequences of the present application may be deoxyribonucleic acid (DNA) sequences or ribonucleic acid (RNA) sequences and may include naturally occurring bases including adenine, guanine, cytosine, thymidine and uracil. The sequences may also contain modified bases. Examples of such modified bases include aza and deaza adenine, guanine, cytosine, thymidine and uracil; and xanthine and hypoxanthine. The nucleic acid can be either double stranded or single stranded, and represents the sense or antisense strand. Further, the term “nucleic acid” includes the complementary nucleic acid sequences.

The term “nucleic acid molecule” or its derivatives, as used herein, is intended to include unmodified DNA or RNA or modified DNA or RNA For example, it may be useful for the nucleic acid molecules of the disclosure to be composed of single- and double-stranded DNA, DNA that is a mixture of single- and double-stranded regions, single- and double-stranded RNA, and RNA that is a mixture of single- and double stranded regions, hybrid molecules comprising DNA and RNA that may be single stranded or, more typically double-stranded or a mixture of single- and double-stranded regions. In addition, it may be useful for the nucleic acid molecules to be composed of triple stranded regions comprising RNA or DNA or both RNA and DNA. The nucleic acid molecules of the disclosure may also contain one or more modified bases or DNA or RNA backbones modified for stability or for other reasons. “Modified” bases include, for example, tritiated bases and unusual bases such as inosine. A variety of modifications can be made to DNA and RNA; thus “nucleic acid molecule” encompasses chemically, enzymatically, or metabolically modified forms. The term “polynucleotide” shall have a corresponding meaning.

The term “isolated polynucleotide” means a polynucleotide which has generally been separated from the polynucleotide sequences with which it is associated or linked in its native state, if the polynucleotide is found in nature. Preferably, the isolated polynucleotide is at least 90% free from other components with which it is naturally associated, if it is found in nature. Preferably the polynucleotide is not naturally occurring, for example by covalently joining two shorter polynucleotide sequences in a manner not found in nature (chimeric polynucleotide).

A genomic form or clone of a gene containing the transcribed region may be interrupted with non-coding sequences termed “introns” or “intervening regions” or “intervening sequences”, which may be either homologous or heterologous with respect to the “exons” of the gene. An “intron” as used herein is a segment of a gene which is transcribed as part of a primary RNA transcript but is not present in the mature mRNA molecule. Introns are removed or “spliced out” from the nuclear or primary transcript; introns therefore are absent in the messenger RNA (mRNA). Introns may contain regulatory elements such as enhancers. “Exons” as used herein refer to the DNA regions corresponding to the RNA sequences which are present in the mature mRNA or the mature RNA molecule in cases where the RNA molecule is not translated. An mRNA functions during translation to specify the sequence or order of amino acids in a nascent polypeptide. The term “gene” includes a synthetic or fusion molecule encoding all or part of the proteins of the invention described herein and a complementary nucleotide sequence to any one of the above.

As used herein, a “chimeric gene” refers to any gene that comprises covalently joined sequences that are not found joined in nature. Typically, a chimeric gene comprises regulatory and transcribed or protein coding sequences that are not found together in nature. Accordingly, a chimeric gene may comprise regulatory sequences and coding sequences that are derived from different sources, or regulatory sequences and coding sequences derived from the same source, but arranged in a manner different than that found in nature. The term “endogenous” is used herein to refer to a mRNA that is normally present in a mammalian cell and refers to a native gene in its natural location in the genome of an organism. It may also be referred to as wild-type. As used herein, “recombinant nucleic acid molecule”, “recombinant polynucleotide” or variations thereof refer to a nucleic acid molecule which has been constructed or modified by recombinant DNA/RNA technology. The terms “foreign polynucleotide” or “exogenous polynucleotide” or “heterologous polynucleotide” and the like refer to any nucleic acid which is introduced into the genome of a cell by experimental manipulations.

Foreign or exogenous genes may be genes that are inserted into a non-native organism or cell, native genes introduced into a new location within the native host, or chimeric genes. Alternatively, foreign or exogenous genes may be the result of editing the genome of the organism or cell, or progeny derived therefrom. A “transgene” is a gene that has been introduced into the genome by a transformation procedure.

The term “genetically engineered”, “genetically modified”, “genetic modification” or variants thereof refers to any genetic manipulation by man and includes introducing genes into cells by transformation or transduction, gene editing, cisgenesis, mutating genes in cells and altering or modulating the regulation of a gene in a cell or organisms to which these acts have been done or their progeny and so on.

Furthermore, the term “exogenous” in the context of a polynucleotide (nucleic acid) refers to the polynucleotide when present in a cell that does not naturally comprise the polynucleotide. The cell may be a cell which comprises a non-endogenous polynucleotide resulting in an altered amount of production of the encoded polypeptide, for example an exogenous polynucleotide which increases the expression of an endogenous polypeptide, or a cell which in its native state does not produce the polypeptide. Increased production of a polypeptide of the invention is also referred to herein as “over-expression”. An exogenous polynucleotide of the invention includes polynucleotides which have not been separated from other components of the transgenic (recombinant) cell, or cell-free expression system, in which it is present, and polynucleotides produced in such cells or cell-free systems which are subsequently purified away from at least some other components. The exogenous polynucleotide (nucleic acid) can be a contiguous stretch of nucleotides existing in nature, or comprise two or more contiguous stretches of nucleotides from different sources (naturally occurring and/or synthetic) joined to form a single polynucleotide. Typically, such chimeric polynucleotides comprise at least an open reading frame encoding a polypeptide of the invention operably linked to a promoter suitable of driving transcription of the open reading frame in a cell of interest.

The % identity of a polynucleotide is determined by GAP (Needleman and Wunsch, 1970) analysis (GCG program) with a gap creation penalty=5, and a gap extension penalty=0.3. The query sequence is at least 900 nucleotides in length, and the GAP analysis aligns the two sequences over a region of at least 900 nucleotides. Preferably, the query sequence is at least 975 nucleotides in length, and the GAP analysis aligns the two sequences over a region of at least 975 nucleotides. Even more preferably, the query sequence is at least 1,050 nucleotides in length and the GAP analysis aligns the two sequences over a region of at least 1,050 nucleotides. Even more preferably, the GAP analysis aligns two sequences over their entire length.

With regard to the defined polynucleotides, it will be appreciated that % identity figures higher than those provided above will encompass preferred embodiments. Thus, where applicable, in light of the minimum % identity figures, it is preferred that the polynucleotide comprises a polynucleotide sequence which is at least 50%, at least 60%, more preferably at least 70%, more preferably at least 75%, more preferably at least 80%, more preferably at least 85%, more preferably at least 90%, more preferably at least 91%, more preferably at least 92%, more preferably at least 93%, more preferably at least 94%, more preferably at least 95%, more preferably at least 96%, more preferably at least 97%, more preferably at least 98%, more preferably at least 99%, more preferably at least 99.1%, more preferably at least 99.2%, more preferably at least 99.3%, more preferably at least 99.4%, more preferably at least 99.5%, more preferably at least 99.6%, more preferably at least 99.7%, more preferably at least 99.8%, and even more preferably at least 99.9% identical to the relevant nominated SEQ ID NO.

When referring to polynucleotide identity herein, it will be understood that a given sequence identity is in reference to the open reading frame sequence.

In a further embodiment, the present invention relates to polynucleotides which are substantially identical or identical to those specifically described herein. As used herein, with reference to a polynucleotide the term “substantially identical” means the substitution of one or a few (for example 2, 3, or 4) nucleotides whilst maintaining at least one activity of the native protein encoded by the polynucleotide. In addition, this term includes the addition or deletion of nucleotides which results in the increase or decrease in size of the encoded native protein by one or a few (for example 2, 3, or 4) amino acids whilst maintaining at least one activity of the native protein encoded by the polynucleotide.

The present invention also relates to the use of oligonucleotides, for instance in methods of screening for a mRNA molecule of the invention. As used herein, “oligonucleotides” are polynucleotides up to 50 nucleotides in length. The minimum size of such oligonucleotides is the size required for the formation of a stable hybrid between an oligonucleotide and a complementary sequence on a nucleic acid molecule of the present invention. They can be RNA, DNA, or combinations or derivatives of either. Oligonucleotides are typically relatively short single stranded molecules of 10 to 30 nucleotides, commonly 15-25 nucleotides in length. When used as a guide for genome editing, probe or as a primer in an amplification reaction, the minimum size of such an oligonucleotide is the size required for the formation of a stable hybrid between the oligonucleotide and a complementary sequence on a target nucleic acid molecule. Preferably, the oligonucleotides are at least 15 nucleotides, more preferably at least 18 nucleotides, more preferably at least 19 nucleotides, more preferably at least 20 nucleotides, more preferably at least 22 nucleotides, even more preferably at least 25 nucleotides in length. Oligonucleotides of the present invention used as a probe are typically conjugated with a label such as a radioisotope, an enzyme, biotin, a fluorescent molecule or a chemiluminescent molecule.

As those skilled in the art would be aware, the sequence of the oligonucleotide primers described herein can be varied to some degree without effecting their usefulness for the methods of the invention. A “variant” of an oligonucleotide disclosed herein (also referred to herein as a “primer” or “probe” depending on its use) useful for the methods of the invention includes molecules of varying sizes of, and/or are capable of hybridising to the genome close to that of, the specific oligonucleotide molecules defined herein. For example, variants may comprise additional nucleotides (such as 1, 2, 3, 4, or more), or less nucleotides as long as they still hybridise to the target region. Furthermore, a few nucleotides may be substituted without influencing the ability of the oligonucleotide to hybridise the target region. In addition, variants may readily be designed which hybridise close (for example, but not limited to, within 50 nucleotides or within 100 nucleotides) to the region of the genome where the specific oligonucleotides defined herein hybridise.

The present invention includes oligonucleotides that can be used as, for example, guides for RNA-guided endonucleases, probes to identify nucleic acid molecules, or primers to produce nucleic acid molecules. Probes and/or primers can be used to clone homologues of the polynucleotides of the invention from other species. Furthermore, hybridization techniques known in the art can also be used to screen genomic or cDNA libraries for such homologues.

Polynucleotides and oligonucleotides of the present invention include those which hybridize under stringent conditions to one or more of the sequences disclosed herein. As used herein, stringent conditions are those that (1) employ low ionic strength and high temperature for washing, for example, 0.015 M NaCl/0.0015 M sodium citrate/0.1% NaDodSO₄ at 50° C.; (2) employ during hybridisation a denaturing agent such as formamide, for example, 50% (vol/vol) formamide with 0.1% bovine serum albumin, 0.1% Ficoll, 0.1% polyvinylpyrrolidone, 50 mM sodium phosphate buffer at pH 6.5 with 750 mM NaCl, 75 mM sodium citrate at 42° C.; or (3) employ 50% formamide, 5×SSC (0.75 M NaCl, 0.075 M sodium citrate), 50 mM sodium phosphate (pH 6.8), 0.1% sodium pyrophosphate, 5×Denhardt's solution, sonicated salmon sperm DNA (50 g/ml), 0.1% SDS and 10% dextran sulfate at 42° C. in 0.2×SSC and 0.1% SDS.

Polynucleotides of the present invention may possess, when compared to naturally occurring molecules, one or more mutations which are deletions, insertions, or substitutions of nucleotide residues. Mutants can be either naturally occurring (that is to say, isolated from a natural source) or synthetic (for example, by performing site-directed mutagenesis on the nucleic acid). A variant of a polynucleotide of the invention includes molecules of varying sizes when compared to the reference polynucleotides defined herein. For example, variants may comprise additional nucleotides (such as 1, 2, 3, 4, or more), or less nucleotides as long as they encode a functional protein. Furthermore, a few nucleotides may be substituted without influencing the integrity of the encoded protein. In addition, variants may include polynucleotides which encode the same polypeptide or amino acid sequence but which vary in nucleotide sequence by redundancy of the genetic code. The terms “polynucleotide variant” and “variant” also include naturally occurring allelic variants.

### Polypeptides

According to the present invention, a mRNA of the invention is designed to encode a polypeptide that is suitable for treating or preventing a lysosomal storage disease, preferably Niemann-Pick type C disease. A polypeptide of interest may include, but is not limited to, whole polypeptides, a plurality of polypeptides or fragments of polypeptides, which independently may be encoded by one or more nucleic acids, a plurality of nucleic acids, fragments of nucleic acids or variants of any of the aforementioned.

As used herein, the term “polypeptide” means a polymer of amino acid residues (natural or unnatural) linked together typically by peptide bonds. The term, as used herein, refers to proteins, polypeptides, and peptides of any size, structure, or function. In some instances the polypeptide encoded is smaller than about 50 amino acids and the polypeptide is then termed a peptide. If the polypeptide is a peptide, it will be at least about 2, 3, 4, or at least 5 amino acid residues long. Thus, polypeptides include gene products, naturally occurring polypeptides, synthetic polypeptides, homologs, orthologs, paralogs, fragments and other equivalents, variants, and analogs of the foregoing. A polypeptide may be a single molecule or may be a multi-molecular complex such as a dimer, trimer or tetramer. They may also comprise single chain or multichain polypeptides and may be associated or linked. Most commonly disulfide linkages are found in multichain polypeptides. The term polypeptide may also apply to amino acid polymers in which one or more amino acid residues are an artificial chemical analogue of a corresponding naturally occurring amino acid.

The term “polypeptide variant” refers to molecules which differ in their amino acid sequence from a native or reference sequence. The amino acid sequence variants may possess substitutions, deletions, and/or insertions at certain positions within the amino acid sequence, as compared to a native or reference sequence. Ordinarily, variants will possess at least about 50% identity (homology) to a native or reference sequence, and preferably, they will be at least about 70%, at least about 80%, at least about 90% identical, at least about 90% identical, at least about 91% identical, at least about 92% identical, at least about 93% identical, at least about 94% identical, at least about 95% identical, at least about 96% identical, at least about 97% identical, at least about 98% identical, at least about 99% identical, or about 100% identical to a native or reference sequence.

A “wildtype NPC1 protein” refers to any protein encoded by a wild-type gene that is capable of having normal (level of function absent disease or disorder) biological activity when expressed in vivo. Such functionality can be tested by any means known to establish functionality of a protein. The term “mutant NPC1 gene” refers to any non-wildtype NPC1 sequence and may include NPC1 gene mutations including Gly248Val, Met1142Thr, Ile1061Thr and Arg404Trp. Typically, a “mutant NPC1 gene” refers to a non-wildtype sequence that results in an abberant functioning NPC1 protein, and thus, NPC disease.

In an embodiment, a codon optimised and/or chemically modified NPC1 mRNA of the invention may encode a polypeptide having a similar protein expression profile when compared to a wild-type NPC1 mRNA counterpart. In another embodiment, a codon optimised and/or chemically modified NPC1 mRNA may encode a polypeptide that rescues a protein insufficiency in cells in a subject with a lysosomal storage disorder, preferably Niemann-Pick type C disease. In another embodiment, a codon optimised and/or chemically modified NPC1 mRNA may encode a polypeptide that reduces free and/or unesterified cholesterol in cells in a manner that is similar to a wild-type NPC1 mRNA counterpart. In another embodiment, a codon optimised and/or chemically modified NPC1 mRNA of the invention may encode a polypeptide that increases esterified cholesterol in cells in a manner that is similar to levels of a wild-type NPC1 mRNA counterpart. Thus, codon optimisation and/or chemical modification of an mRNA of the invention can provide for protein function that is similar to that of the wild-type NPC1 protein counterpart. In yet additional embodiments, a mRNA of the invention may be compared to a “corresponding mRNA” that has already been optimised, such as that defined in SEQ ID NO: 2. Typically, polypeptide encoded by the the codon optimised and/or chemically modified NPC1 mRNA has a better protein expression profile, rescues a protein insufficiency, reduces free and/or unesterified cholesterol, increases esterified cholesterol or improves protein function to a greater extent than a mutant NPC1 gene, for instance a NPC1 gene containing a Gly248Val, Met1142Thr, Ile1061Thr or Arg404Trp mutation.

The protein encoded by the the codon optimised and/or chemically modified NPC1 mRNA is generally about 30 to about 60, from about 60 to about 100, from about 100 to about 130, from about 130 to about 160, from about 160 to about 200, from about 200 to about 230, from about 230 to about 260, from about 260 to about 300, from about 300 to about 330, from about 330 to about 400, from about 400 to about 460, from about 460 to about 530, from about 530 to about 600, from about 600 to about 660, from about 660 to about 800, from about 800 to about 930, from about 930 to about 1000, from about 1000 to about 1060, from about 1060 to about 1130, from about 1130 to about 1200, from about 1200 to about 1260, from about 1260 to about 1330, from about 1330 to about 1400 or more amino acids. Preferably, the polypeptide encoded by the mRNA molecule is about 1260 or about 166 amino acids, more preferably the polypeptide is encoded by the mRNA sequence set forth in SEQ ID NO: 2.

In an embodiment, the polypeptide encoded by a mRNA of the invention may have a function that is from at least about 60-65%, from at least about 65-70%, from at least about 70-75%, from at least about 75-80%, from at least about 80-85%, from at least about 85-90%, from at least about 90-95% from at least about 95-100%, from at least about 105-110%, from at least about 110-115%, from at least about 115-120%, from at least about 120-125%, from at least about 125-130%, from at least about 130-135%, from at least about 135-140%, from at least about 145-150%, from at least about 155-160%, from at least about 160-165%, from at least about 165-170%, from at least about 170-175%, from at least about 175-180%, from at least about 180-185%, from at least about 185-190%, from at least about 190-195%, from at least about 195-200% or more compared to a corresponding wild-type polypeptide. Preferaby the polypeptide encoded by a mRNA of the invention has a function that is about the same as a wild-type counterpart. The corresponding wild-type polypeptide may be a sequence set forth in SEQ ID NO: 10 or 11.

The term “NPC1 protein” or “NPC1 polypeptide” refers to any wildtype amino acid NPC1 sequence or fragment thereof. Typically, a “mutant NPC1 protein” refers to a non-wildtype NPC1 polypeptide that has an abberant function as compared to a wildtype NPC1 protein, and which results in NPC disease. The term “NPC2 protein” or “NPC2 polypeptide” refers to any wildtype amino acid NPC2 sequence or fragment thereof. Typically, a “mutant NPC2 protein” refers to a non-wildtype NPC2 polypeptide that has an abberant function as compared to a wildtype NPC2 protein, and which results in NPC disease.

The NPC1 protein may be functionally characterized by its ability, when expressed in target cells, including those of the nervous system, liver and lungs, to correct lysosomal cholesterol accumulation or cholesterol unesterification that is characteristic of a lysosomsal storage disorder such as Niemann-Pick type C disease. Methods for determining levels of total cholesterol, esterified cholesterol or unesterified cholesterol are known in the art and described herein including in the Example. In brief, unesterified cholesterol in cell samples can be oxidized by cholesterol oxidase to yield hydrogen peroxide, levels of which can be detected using a hydrogen peroxide-specific probe that produces fluorescence in response to the reaction. Esterified cholesterol can be hydrolyzed by cholesterol esterase into cholesterol, and thus quantified in the same way. The amount of esterified cholesterol in a cell can be determined by subtracting the amount of free cholesterol from the amount of total cholesterol (cholesterol plus cholesterol esters). Other bioluminescence-based methods of quantifying esterified and unesterified cholesterol levels also exist. In addition, cholesterol-binding fluorescent probes (e.g., filipin), and lysosome-labelling dyes (e.g., LysoTracker Red DND-99) can be used to visualize and thus quantify the amount of unesterified cholesterol in a cell. Lysosome-labelling dyes can also be used to quantify lysosome size, which often increases in NPC disease.

“Identity” or “homology” as it applies to amino acid sequences is defined as the percentage of residues in the candidate amino acid sequence that are identical with the residues in the amino acid sequence of a second sequence after aligning the sequences and introducing gaps, if necessary, to achieve the maximum percent homology. Methods and computer programs for the alignment are well known in the art. It is understood that homology depends on a calculation of percent identity but may differ in value due to gaps and penalties introduced in the calculation.

By “homologs” as it applies to polypeptide sequences means the corresponding sequence of other species having substantial identity to a second sequence of a second species.

“Analog” is meant to include a polypeptide variant which differ by one or more amino acid alterations, e.g., substitutions, additions or deletions of amino acid residues that still maintain one or more of the properties of the parent or starting polypeptide.

mRNA encoding polypeptides containing substitutions, insertions and/or additions, deletions and covalent modifications with respect to reference sequences, in particular the polypeptide sequences disclosed herein, are included within the scope of this invention. For example, sequence tags or amino acids, such as one or more lysines, can be added to the peptide sequences of the invention (e.g., at the N-terminal or C-terminal ends).

Sequence tags can be used for peptide purification or localization. Lysines can be used to increase peptide solubility or to allow for biotinylation. Alternatively, amino acid residues located at the carboxy and amino terminal regions of the amino acid sequence of a peptide or protein may optionally be deleted providing for truncated sequences. Certain amino acids (e.g., C-terminal or N-terminal residues) may alternatively be deleted depending on the use of the sequence, as for example, expression of the sequence as part of a larger sequence which is soluble, or linked to a solid support.

“Substitutional variants” when referring to polypeptides are those that have at least one amino acid residue in a native or starting sequence removed and a different amino acid inserted in its place at the same position. The substitutions may be single, where only one amino acid in the molecule has been substituted, or they may be multiple, where two or more amino acids have been substituted in the same molecule.

As used herein the term “conservative amino acid substitution” refers to the substitution of an amino acid that is normally present in the sequence with a different amino acid of similar size, charge, or polarity. Examples of conservative substitutions include the substitution of a non-polar (hydrophobic) residue such as isoleucine, valine and leucine for another non-polar residue. Likewise, examples of conservative substitutions include the substitution of one polar (hydrophilic) residue for another such as between arginine and lysine, between glutamine and asparagine, and between glycine and serine.

Additionally, the substitution of a basic residue such as lysine, arginine or histidine for another, or the substitution of one acidic residue such as aspartic acid or glutamic acid for another acidic residue are additional examples of conservative substitutions. Examples of non-conservative substitutions include the substitution of a non-polar (hydrophobic) amino acid residue such as isoleucine, valine, leucine, alanine, methionine for a polar (hydrophilic) residue such as cysteine, glutamine, glutamic acid or lysine and/or a polar residue for a non-polar residue.

As used herein the terms “termini” or “terminus” when referring to polypeptides refers to an extremity of a peptide or polypeptide. Such extremity is not limited only to the first or final site of the peptide or polypeptide but may include additional amino acids in the terminal regions. The polypeptide based molecules of the present invention may be characterized as having both an N-terminus (terminated by an amino acid with a free amino group (NH2)) and a C-terminus (terminated by an amino acid with a free carboxyl group (COOH)). Proteins of the invention are in some cases made up of multiple polypeptide chains brought together by disulfide bonds or by non-covalent forces (multimers, oligomers). These sorts of proteins will have multiple N- and C-termini. Alternatively, the termini of the polypeptides may be modified such that they begin or end, as the case may be, with a nonpolypeptide based moiety such as an organic conjugate.

Once any of the features have been identified or defined as a desired component of a polypeptide to be encoded by the mRNA of the invention, any of several manipulations and/or modifications of these features may be performed by moving, swapping, inverting, deleting, randomizing or duplicating. Furthermore, it is understood that manipulation of features may result in the same outcome as a modification to the molecules of the invention. For example, a manipulation which involved deleting a domain would result in the alteration of the length of a molecule just as modification of a nucleic acid to encode less than a full length molecule would.

Modifications and manipulations can be accomplished by methods known in the art such as, but not limited to, site directed mutagenesis. The resulting modified molecules may then be tested for activity using in vitro or in vivo assays such as those described herein or any other suitable screening assay known in the art.

According to the present invention, the polypeptides may comprise a consensus sequence which is discovered through rounds of experimentation. As used herein a “consensus” sequence is a single sequence which represents a collective population of sequences allowing for variability at one or more sites.

As recognized by those skilled in the art, protein fragments, functional protein domains, and homologous proteins are also considered to be within the scope of polypeptides of interest of this invention. For example, provided herein is any protein fragment (meaning a polypeptide sequence at least one amino acid residue shorter than a reference polypeptide sequence but otherwise identical) of a reference protein 10, 20, 30, 40, 50, 60, 70, 80, 90, 100 or greater than 100 amino acids in length. In another example, any protein that includes a stretch of about 20, about 30, about 40, about 50, or about 100 amino acids which are about 40%, about 50%, about 60%, about 70%, about 80%, about 90%, about 95%, or about 100% identical to any of the sequences described herein can be utilized in accordance with the invention. In certain embodiments, a polypeptide to be utilized in accordance with the invention includes 2, 3, 4, 5, 6, 7, 8, 9, 10, or more mutations as shown in any of the sequences provided or referenced herein.

The term “identity” as known in the art, refers to a relationship between the sequences of two or more peptides, as determined by comparing the sequences. In the art, identity also means the degree of sequence relatedness between peptides, as determined by the number of matches between strings of two or more amino acid residues. Identity measures the percent of identical matches between the smaller of two or more sequences with gap alignments (if any) addressed by a particular mathematical model or computer program (i.e., “algorithms”). Identity of related peptides can be readily calculated by known methods. Such methods include, but are not limited to, those described in Lesk (1988); Smith (1993); Griffin and Griffin, (1994); von Heinje (1987); Gribskov and Devereux (1991); and Carillo (1988).

In some embodiments, the polypeptide variant may have the same or a similar activity as the reference polypeptide. Alternatively, the variant may have an altered activity (e.g., increased or decreased) relative to a reference polypeptide. In preferred embodiments, the reference polypeptide may be that set forth in SEQ ID NO: 10 or 11.

Generally, variants of a particular polynucleotide or polypeptide of the invention will have at least about 40%, 45%, 50%, 55%, 60%, 65%, 70%, 75%, 80%, 85%, 90%, 91%, 92%, 93%, 94%, 95%, 96%, 97%, 98%, 99% but less than 100% sequence identity to that particular reference polynucleotide or polypeptide as determined by sequence alignment programs and parameters described herein and known to those skilled in the art. Such tools for alignment include those of the BLAST suite (Altschul (1997)) Other tools are described herein, specifically in the definition of “Identity.”

Default parameters in the BLAST algorithm include, for example, an expect threshold of 10, Word size of 28, Match/Mismatch Scores 1, −2, Gap costs Linear. Any filter can be applied as well as a selection for species specific repeats, e.g., Homo sapiens.

The % identity of a polypeptide is determined by GAP (Needleman and Wunsch (1970)) analysis (GCG program) with a gap creation penalty=5, and a gap extension penalty=0.3. The query sequence is at least 100 amino acids in length, and the GAP analysis aligns the two sequences over a region of at least 100 amino acids. More preferably, the query sequence is at least 300 amino acids in length and the GAP analysis aligns the two sequences over a region of at least 300 amino acids. Alternatively, the query sequence is at least 500 amino acids in length and the GAP analysis aligns the two sequences over a region of at least 500 amino acids. Even more preferably, the GAP analysis aligns two sequences over their entire length of any amino acid sequence disclosed herein.

With regard to a defined polypeptide, it will be appreciated that % identity figures higher than those provided above will encompass preferred embodiments. Thus, where applicable, in light of the minimum % identity figures, it is preferred that the polypeptide comprises an amino acid sequence which is preferably at least 50%, at least 60%, at least 70%, more preferably at least 75%, more preferably at least 76%, more preferably at least 80%, more preferably at least 85%, more preferably at least 90%, more preferably at least 91%, more preferably at least 92%, more preferably at least 93%, more preferably at least 94%, more preferably at least 95%, more preferably at least 96%, more preferably at least 97%, more preferably at least 98%, more preferably at least 99%, more preferably at least 99.1%, more preferably at least 99.2%, more preferably at least 99.3%, more preferably at least 99.4%, more preferably at least 99.5%, more preferably at least 99.6%, more preferably at least 99.7%, more preferably at least 99.8%, and even more preferably at least 99.9% identical to the relevant nominated SEQ ID NO.

### Cells

In an aspect of the invention, there is provided a mammalian cell comprising a mRNA of the invention. As relevant to NPC disease, accumulation of intracellular lipid e.g., unesterified cholesterol is known to occur in cells of internal organs throughout the body including the liver, spleens and lungs. Typically, lipid accumulates in lysosomes of hepatocytes of the liver, lysosomes of reticular cells and lymphocytes of the spleen, and/or lysosomes of alveolar epithelial cells of the lungs. However the predominant symptoms are observed in the nervous system of the subject where intracellular lipid accumulates within various cell types including neurons, microglia, astrocytes and oligodendrocytes. The cell is therefore preferably a hepatocyte, reticular cell, lymphocyte, alveolar epithelial cell, neuronal cell or glial cell.

Moreover, the cell is preferably a human cell but may be any mammalian cell including those from pets (i.e., cats, rabbits, guinea pigs, ferrets, dogs) or livestock animals (cattle, pigs, sheep, goats, chickens, camelids, deer, bison, buffalo and related species including wild and zoo animals).

### Pharmaceutical Compositions

In an aspect of the invention, there is provided a pharmaceutical composition comprising a mRNA of the invention and a pharmaceutically acceptable carrier, diluent or excipient. In some examples, a mRNA described herein can be administered orally, parenterally, by inhalation spray, adsorption, absorption, topically, rectally, nasally, bucally, vaginally, intraventricularly, via an implanted reservoir in dosage formulations containing conventional non-toxic pharmaceutically-acceptable carriers, or by any other convenient dosage form. The term “parenteral” as used herein includes subcutaneous, intravenous, intramuscular, intraperitoneal, intrathecal, intraventricular, intrasternal, and intracranial injection or infusion techniques.

Methods for preparing a mRNA into a suitable form for administration to a subject (e.g. a pharmaceutical composition) are known in the art and include, for example, methods as described in Remington and Gennaro (1990) and U.S. Pharmacopeia: National Formulary (1984).

The pharmaceutical compositions of this invention are useful for parenteral administration, such as intravenous administration or administration into a body cavity or lumen of an organ or joint. The compositions for administration will commonly comprise a solution of a mRNA dissolved in a pharmaceutically acceptable carrier, for example an aqueous carrier. A variety of aqueous carriers can be used, e.g., buffered saline and the like. The compositions may contain pharmaceutically acceptable auxiliary substances as required to approximate physiological conditions such as pH adjusting and buffering agents, toxicity adjusting agents and the like, for example, sodium acetate, sodium chloride, potassium chloride, calcium chloride, sodium lactate and the like. The concentration of a mRNA of the present invention in these formulations can vary widely, and will be selected primarily based on fluid volumes, viscosities, body weight and the like in accordance with the particular mode of administration selected and the patient's needs. Exemplary carriers include water, saline, Ringer's solution, dextrose solution, and 5% human serum albumin. Nonaqueous vehicles such as mixed oils and ethyl oleate may also be used. Liposomes may also be used as carriers. The vehicles may contain minor amounts of additives that enhance isotonicity and chemical stability, e.g., buffers and preservatives.

In certain preferred embodiments, the compositions of the present invention are administered intracranially, for instance injected into the brain, such as by direct injection into the brain. Direct injection may be performed by intraventricular and intracerebral routes. Injection of the compositions into the brain can also be performed using a device for administration. Direct administration of the drugs into the central nervous system may also be achieved by using epidural (injection or infusion into the epidural space), intracerebral (into the cerebrum), intracerebroventricular (into the cerebral ventricles), or intrathecal (into the spinal canal) injection. Pathan et al. (2009), incorporated by reference in its entirety herein, describes some methods of administration of a composition to the brain and others are known in the art. Alternatively, the compositions of the present invention may be administered to the central nervous system by systemic delivery, preferably by intravenous delivery.

In an embodiment of the invention, an mRNA of the invention may be formulated in a liposome, lipoplex or a lipid nanoparticle so as to increase stability of the mRNA. Liposomes are artificially-prepared vesicles which are primarily be composed of a lipid bilayer and may be used as a delivery vehicle for the administration of nutrients and pharmaceutical formulations. Liposomes can be of different sizes such as, but not limited to, a multilamellar vesicle (MLV) which may be hundreds of nanometers in diameter and may contain a series of concentric bilayers separated by narrow aqueous compartments, a small unicellular vesicle (SUV) which may be smaller than 50 nm in diameter, and a large unilamellar vesicle (LUV) which may be between 50 and 500 nm in diameter.

Liposomes, lipoplexes or lipid nanoparticles may include, but is not limited to, opsonins or ligands in order to improve the attachment of liposomes, lipoplex or lipid nanoparticles to unhealthy tissue or to activate events such as, but not limited to, endocytosis. Liposomes, lipoplexes or lipid nanoparticles may contain a low or a high pH in order to improve the delivery of the pharmaceutical formulations. The formation of liposomes, lipoplexes or lipid nanoparticles may depend on the physicochemical characteristics such as, but not limited to, the pharmaceutical formulation entrapped and the liposomal ingredients, the nature of the medium in which the lipid vesicles are dispersed, the effective concentration of the entrapped substance and its potential toxicity, any additional processes involved during the application and/or delivery of the vesicles, the optimization size, polydispersity and the shelf-life of the vesicles for the intended application, and the batch-to-batch reproducibility and possibility of large-scale production of safe and efficient liposome, lipoplexe or lipid nanoparticle products.

Preferably, the mRNA of the invention is formulated a lipid nanoparticle. LNPs are composed primarily of cationic lipids along with other lipid ingredients. These typically include neutral phospholipid molecules belonging to the phosphatidylcholine (PC) class and sterols, such as cholesterol. Another common lipid ingredient is what is known as a PEGylated phospholipid—a polyethylene glycol (PEG) polymer covalently attached to the head-group of a phospholipid.

As used herein, the term “lipid nanoparticle” or “LNP” shall therefore be understood to refer to lipid-based particles having at least one dimension on the order of nanometers (e.g., 1-1,000 nm) and which typically comprises a mRNA described herein. In embodiments, LNPs are formulated in a composition for delivery of a mRNA to a desired target such as a cell, tissue, organ, tumor, and the like. For example, the lipid nanoparticle or LNP may be any lipid composition, including, may be selected from, but not limited to, liposomes or vesicles, where an aqueous volume is encapsulated by amphipathic lipid bilayers (e.g., single; unilamellar or multiple; multilamellar), micelle-like lipid nanoparticles having a non-aqueous core and solid lipid nanoparticles, wherein solid lipid nanoparticles lack lipid bilayers.

Whilst liposomes include one or more rings of lipid bilayer surrounding an aqueous pocket, not all lipid nanoparticles have a contiguous bilayer like liposomes. Instead, it is understood that some LNPs assume a micelle-like structure, encapsulating drug molecules in a non-aqueous core. Where the use of lipid nanoparticles is contemplated, a lipid nanoparticle generally comprises a cationic lipid, a non-cationic lipd, a PEG lipid and a structural lipid. Suitable cationic lipids may include those described in the cationic lipid may be selected from, but not limited to, a cationic lipid described in International Publication Nos. WO2012040184, WO2011153120, WO2011149733, WO2011090965, WO2011043913, WO2011022460, WO2012061259, WO2012054365, WO2012044638, WO2010080724, WO201021865, WO2008103276, WO2013086373 and WO2013086354, U.S. Pat. Nos. 7,893,302, 7,404,969, 8,283,333, and 8,466,122 and US Patent Publication No. US20100036115, US20120202871, US20130064894, US20130129785, US20130150625, US20130178541 and US20130225836. Other suitable cationic lipids, non-cationic lipids, PEG lipids and structural lipids, and suitable ratios thereof include those disclosed in WO 2015164674 and WO 2013090648. For example, mRNA according to the invention may be formulated in a lipid nanoparticle at a 20:1 weight ratio of total lipid to modified mRNA with preferred lipid molar ratios of DLin-KC2-DMA: DSPC: Cholesterol: PEG-c-DOMG.

Exemplary lipid nanoparticle compositions and methods of making same are described, for example, in Semple et al. (2010); Jayarama et al. (2012); and Maier et al. (2013). Alternatively, the LNP formulation may be formulated by the methods described in International Publication Nos. WO2011127255 or WO2008103276.

Further, the particle size of the lipid nanoparticle may be increased and/or decreased. The change in particle size may be able to help counter biological reaction such as, but not limited to, inflammation or may increase the biological effect of the modified mRNA delivered to a given subject.

Lipid nanoparticles suitable for use in the present disclosure will be apparent to the skilled person and/or are described herein. The lipids can have an anionic, cationic or zwitterionic hydrophilic head group.

In one embodiment, the lipid nanoparticle comprises a PEG-lipid, a sterol structural lipid and/or a neutral lipid. In one example, the lipid nanoparticle further comprises a cationic lipid. In one example, the lipid nanoparticle does not comprise a cationic lipid.

In one embodiment, the LNP comprises a PEG-lipid. For example, the PEG-lipid is selected from the group consisting of PEG-c-DMG, PEG-DMG, PEG-DLPE, PEG-DMPE, PEG-DPPC, a PEG-DSPE lipid and combinations thereof.

In one embodiment, the LNP comprises a structural lipid. For example, the structural lipid is selected from the group consisting of cholesterol fecosterol, sitosterol, campesterol, stigmasterol, brassicasterol, ergosterol, tomatidine, tomatine, ursolic acid and alpha-tocopherol and combinations thereof.

In one embodiment, the LNP comprises a neutral lipid. Exemplary phospholipids (anionic or zwitterionic) for use in the present disclosure include, for example, phosphatidylethanolamines, phosphatidylcholines, phosphatidylserines, and phosphatidylglycerols. For example, the neutral lipid is selected from the group consisting of 1,2-distearoyl-sn-glycero-3-phosphocholine (DSPC), 1,2-dioleoyl-sn-glycero-3-phosphoethanolamine (DOPE), 1,2-dilinoleoyl-sn-glycero-3-phosphocholine (DLPC), 1,2-dimyristoyl-sn-glycero-phosphocholine (DMPC), 1,2-dioleoyl-sn-glycero-3-phosphocholine (DOPC), 1,2-dipalmitoyl-sn-glycero-3-phosphocholine (DPPC), 1,2-diundecanoyl-sn-glycero-phosphocholine (DUPC), 1-palmitoyl-2-oleoyl-sn-glycero-3-phosphocholine (POPC), 1,2-di-O-octadecenyl-sn-glycero-3-phosphocholine (18:0 Diether PC), 1-oleoyl-2-cholesterylhemisuccinoyl-sn-glycero-3-phosphocholine (OChemsPC), 1-hexadecyl-sn-glycero-3-phosphocholine (C16 Lyso PC), 1,2-dilinolenoyl-sn-glycero-3-phosphocholine, 1,2-diarachidonoyl-sn-glycero-3-phosphocholine, 1,2-didocosahexaenoyl-sn-glycero-3-phosphocholine, 1,2-diphytanoyl-sn-glycero-3-phosphoethanolamine (ME 16.0 PE), 1,2-distearoyl-sn-glycero-3-phosphoethanolamine (DSPE), 1,2-dilinoleoyl-sn-glycero-3-phosphoethanolamine, 1,2-dilinolenoyl-sn-glycero-3-phosphoethanolamine, 1,2-diarachidonoyl-sn-glycero-3-phosphoethanolamine, 1,2-didocosahexaenoyl-sn-glycero-3-phosphoethanolamine, 1,2-dioleoyl-sn-glycero-3-phospho-rac-(1-glycerol) sodium salt (DOPG), and sphingomyelin and combinations thereof.

In one embodiment, the LNP comprises a cationic lipid. Exemplary cationic lipids include, but are not limited to, dioleoyl trimethylammonium propane (DOTAP), 1,2-distearyloxy-N,N-dimethyl-3-aminopropane (DSDMA), 1,2-dioleyloxy-N,Ndimethyl-3-aminopropane (DODMA), 1,2-dilinoleyloxy-N,N-dimethyl-3-aminopropane (DLinDMA), 1,2-dilinolenyloxy-N,N-dimethyl-3-aminopropane (DLenDMA), 2,5-bis((9z,12z)-octadeca-9,12,dien-1-yloxyl)benzyl-4-(dimethylamino)butnoate (LKY750). In one example, the phospholipid is 2,5-bis((9z,12z)-octadeca-9,12,dien-1-yloxyl)benzyl-4-(dimethylamino)butnoate (LKY750). Exemplary zwitterionic lipids include, but are not limited to, acyl zwitterionic lipids and ether zwitterionic lipids, such as dipalmitoylphosphatidylcholine (DPPC), dioleoylphosphatidylcholine (DOPC) and dodecylphosphocholine. The lipids can be saturated or unsaturated.

In one example, the pharmaceutical composition of the present disclosure further comprises a polymeric microparticle.

The skilled person will be aware that various polymers can form microparticles to encapsulate or adsorb the mRNA of the present disclosure. It will be apparent that use of a substantially non-toxic polymer means that particles are safe, and the use of a biodegradable polymer means that the particles can be metabolised after delivery to avoid long-term persistence. Useful polymers are also sterilisable, to assist in the preparation of pharmaceutical grade formulations.

Exemplary non-toxic and biodegradable polymers include, but are not limited to, poly(α-hydroxy acids), polyhydroxy butyric acids, polylactones (including polycaprolactones), polydioxanones, polyvalerolactone, polyorthoesters, polyanhydrides, polycyanoacrylates, tyrosine-derived polycarbonates, polyvinyl-pyrrolidinones or polyester-amides, and combinations thereof.

In embodiment, the pharmaceutical composition of the present disclosure further comprises an oil-in-water cationic emulsion.

Suitable oils for use in an oil-in-water emulsion will be apparent to the skilled person and/or are described herein. For example, the emulsion comprises one or more oils derived, for example, from an animal (e.g., fish) or a vegetable source (e.g., nuts, seeds, grains). The skilled person will recognise that biocompatible and biodegradable oils are preferentially used. Exemplary animal oils (i.e., fish oils) include cod liver oil, shark liver oils, and whale oil. Exemplary vegetable oils include peanut oil, coconut oil, olive oil, soybean oil, jojoba oil, safflower oil, cottonseed oil, sunflower seed oil, sesame seed oil, corn oil.

In addition to the oil, the oil-in-water emulsion also comprises a cationic lipid to facilitate formation and stabilisation of the emulsion. Suitable cationic lipids will be apparent to the skilled person and/or are described herein. Exemplary cationic lipids include, but are not limited to, limited to: 1,2-dioleoyloxy-3-(trimethylammonio) propane (DOTAP), 3′-[N-(N′,N′-Dimethylaminoethane)-carbamoyl] Cholesterol (DC Cholesterol), dimethyldioctadecyl-ammonium (DDA), 1,2-Dimyristoyl-3-Trimethyl-AmmoniumPropane (DMTAP), dipalmitoyl[C16:0]trimethyl ammonium propane (DPTAP) and distearoyltrimethylammonium propane (DSTAP).

In some embodiments, the oil-in-water emulsion also comprises a non-ionic surfactant and/or a zwitterionic surfactant. The skilled person will be aware of surfactants suitable for use in the present disclosure. Exemplary surfactants include, but are not limited to: the polyoxyethylene sorbitan esters surfactants (e.g., polysorbate 20 and polysorbate 80) and copolymers of ethylene oxide (EO), propylene oxide (PO), and/or butylene oxide (BO).

Where delivery across the blood brain barrier is contemplated, the formulation may comprise one or more components to aid delivery across the blood brain barrier. In an embodiment, there is therefore provided LNP formulations that additionally comprise a permeability enhancer molecule. Non-limiting permeability enhancer molecules are described in US Patent Publication No. US20050222064.

In certain embodiments, the target site can be located any region of the CNS, including the brain and the spinal cord. A site of mRNA administration within the CNS can be chosen based on the desired target region of neuropathology and, optionally, the topology of brain circuits involved when an administration site and the target region have axonal connections. In certain embodiments, the target region can be defined, for example, using 3-D stereotaxic coordinates. An administration site may be localized in a region innervated by projection neurons connecting distal regions of the brain. For example, the substantia nigra and ventral tegmental area send dense projections to the caudate and putamen (collectively known as the striatum). Neurons within the substantia nigra and ventral tegmentum can be targeted for transduction by retrograde transport of a mRNA following injection into the striatum. As another example, the hippocampus receives well-defined, predictable axonal projections from other regions of the brain. Other administration sites may be localized, for example, in the spinal cord, brainstem (medulla and pons), mesencephalon, cerebellum, diencephalon (thalamus, hypothalamus), telencephalon (corpus striatum, cerebral cortex, or, within the cortex, the occipital, temporal, parietal or frontal lobes), or combinations thereof.

To deliver a mRNA described herein specifically to a particular region of the central nervous system, especially to a particular region of the brain, it may be administered by stereotaxic microinjection. For example, on the day of surgery, patients will have the stereotaxic frame base fixed in place (screwed into the skull). The brain with stereotaxic frame base (MM-compatible with fiduciary markings) will be imaged using high resolution MM. The MM images will then be transferred to a computer that runs stereotaxic software. A series of coronal, sagittal and axial images will be used to determine the target site of vector injection, and trajectory. The software directly translates the trajectory into 3-dimensional coordinates appropriate for the stereotaxic frame. Burr holes are drilled above the entry site and the stereotaxic apparatus localized with the needle implanted at the given depth. The pharmaceutical composition or mRNA will then be administrated by direct injection to the primary target site and retrogradely transported to distal target sites via axons. Additional routes of administration may be used, e.g., intrathecal injection, superficial cortical application under direct visualization, or other non-stereotaxic application.

In another aspect, the invention provides a method of delivering pharmaceutical composition or mRNA to a target cell of the CNS, which is a neuron or a microglia, in a subject with a cholesterol storage disease or disorder, e.g., Niemann-Pick disease, type C. The method comprises contacting an axonal ending of a neuron with a pharmaceutical composition or mRNA; allowing the pharmaceutical composition or mRNA to be endocytosed and retrogradely transported intracellularly along the axon to the nucleus of the neuron; allowing the polypeptide to be expressed and transported within the membrane(s) of the neuron, wherein the polypeptide is therefore capable of treating a cholesterol storage disorder (i.e., NPC).

Optionally, non-CNS delivery can also be performed, e.g., for cholesterol storage diseases or disorders where non-CNS delivery would also be desirable. Such non-CNS delivery of the compositions or mRNA of the instant invention can be performed in addition to or as an alternative to CNS delivery. In certain such embodiments, injection, e.g., intravenous, intraperitoneal, etc. injection can be performed using the compositions of the instant inventtion. Direct delivery to large peripheral nerves is also considered.

Upon formulation, a mRNA of the present invention will be administered in a manner compatible with the dosage formulation and in such amount as is therapeutically effective. Formulations are easily administered in a variety of dosage forms, such as the type of injectable solutions described above, but other pharmaceutically acceptable forms are also contemplated, e.g., tablets, pills, capsules or other solids for oral administration, suppositories, pessaries, nasal solutions or sprays, aerosols, inhalants, liposomal forms and the like. Pharmaceutical “slow release” capsules or compositions may also be used. Slow release formulations are generally designed to give a constant drug level over an extended period and may be used to deliver an antigen binding site of the present invention.

Suitable dosages of a mRNA of the present invention will vary depending on the specific mRNA and/or the subject being treated. It is within the ability of a skilled physician to determine a suitable dosage, e.g., by commencing with a sub-optimal dosage and incrementally modifying the dosage to determine an optimal or useful dosage. Alternatively, to determine an appropriate dosage for treatment/prophylaxis, data from the cell culture assays or animal studies are used, wherein a suitable dose is within a range of circulating concentrations that include the ED50 of the active compound with little or no toxicity. The dosage may vary within this range depending upon the dosage form employed and the route of administration utilized. A therapeutically effective dose can be estimated initially from cell culture assays. A dose may be formulated in animal models to achieve a circulating plasma concentration range that includes the IC50 (i.e., the concentration or amount of the compound which achieves a half-maximal inhibition of symptoms) as determined in cell culture. Such information can be used to more accurately determine useful doses in humans. Levels in plasma maybe measured, for example, by high performance liquid chromatography.

In some examples, a method of the present invention comprises administering a therapeutically effective amount of a mRNA or pharmaceutical composition described herein.

The term “therapeutically effective amount” is the quantity which, when administered to a subject in need of treatment, improves the prognosis and/or state of the subject and/or that reduces or inhibits one or more symptoms of a clinical condition described herein to a level that is below that observed and accepted as clinically diagnostic or clinically characteristic of that condition. The amount to be administered to a subject will depend on the particular characteristics of the condition to be treated, the type and stage of condition being treated, the mode of administration, and the characteristics of the subject, such as general health, other diseases, age, sex, genotype, and body weight. A person skilled in the art will be able to determine appropriate dosages depending on these and other factors. Accordingly, this term is not to be construed to limit the present invention to a specific quantity, e.g., weight or amount of protein(s), rather the present invention encompasses any amount of the antigen binding site(s) sufficient to achieve the stated result in a subject.

The present invention provides for both prophylactic and therapeutic methods of treating a subject at risk of (or susceptible to) a disease or disorder caused, in whole or in part, by altered cholesterol storage, optionally treatable via tissue specific or systemic delivery of a NPC1- and/or NPC2 mRNA of the invention or a pharmaceutical composition thereof.

The term “prophylactically effective amount” shall be taken to mean a sufficient quantity of a mRNA that prevents or inhibits or delays the onset of one or more detectable symptoms of a clinical condition (i.e., NPC). The skilled person will be aware that such an amount will vary depending on, for example, the specific mRNA of the invention or a pharmaceutical composition thereof administered and/or the particular subject and/or the type or severity or level of condition and/or predisposition (genetic or otherwise) to the condition. Accordingly, this term is not to be construed to limit the present invention to a specific quantity, e.g., weight or amount of mRNA of the invention or a pharmaceutical composition thereof, rather the present invention encompasses any amount of the mRNA of the invention or a pharmaceutical composition thereof sufficient to achieve the stated result in a subject.

As used herein, a subject “at risk” of developing a lysosomal storage disease, preferably Niemann-Pick disease, type C may have or may not have detectable symptoms of a lysosomal storage disease, and may have or may not have displayed detectable symptoms of a lysosomal storage disease prior to a treatment according to the present disclosure. “At risk” denotes that a subject has one or more risk factors, which are measurable parameters that correlate with development of the a lysosomal storage disease, as known in the art and/or described herein.

As used herein, “preventing” or “prevention” is intended to refer to at least the reduction of likelihood of the risk of (or susceptibility to) acquiring a disease or disorder (i.e., causing at least one of the clinical symptoms of the disease not to develop in a patient that may be exposed to or predisposed to the disease but does not yet experience or display symptoms of the disease). Biological and physiological parameters for identifying such patients are provided herein and are also well known by physicians. In relation to the present disclosure, prevention of Niemann-Pick disease, type C may be obtained where a subject is identified as having a NPC1 mutation, as disclosed herein, which is associated with the development of Niemann-Pick disease, type C, but does not have any clinical symptoms of the disease and may be considered asymptomatic.

### Methods of Treatment

In an aspect of the invention, there is provided methods of treating a lysosomal storage disease (LSD) in a subject in need thereof with a mRNA of the invention. Lysosomal storage diseases include more than 70 inherited metabolic disorders generally caused by defects in lysosomal function. The incidence is about 1:5000-1:10,000 as a group of diseases.

The term “lysosomal” refers to a recycling center in which cell membrane and other materials break down to small molecules for reuse. Deficiency of a single enzyme or protein required for the metabolism or trafficking of lipids, glycoproteins and other macromolecules can result in lipid accumulation in lysosomes of cells. Excessive amount of lipids or other materials in lysosomes can cause enlargement of internal organs including the liver and spleen. Symptoms of neuronal degeneration are also common clinical manifestations in patients with a lysosomal storage disease.

The lysosomal storage disease to be treated herein by a mRNA or pharmaceutical composition of the invention may include any lysosomal storage disease described herein or known in the art, particularly those associated with compromised or deficient intracellular lipid trafficking.

Non-limiting examples of lysosomal storage disorders to be treated may include Aspartylglucosaminuria, Fabry disease, Infantile neuronal ceroid lipofuscinosis (CNL1), Late Infantile neuronal ceroid lipofuscinosis (CNL2), Juvenile neuronal ceroid lipofuscinosis (CNL3), Adult neuronal ceroid lipofuscinosis disease (CNL4), Finnish/late infantile (CNL5), late infantile variant (CNL6), Type 7 neuronal ceroid lipofuscinosis (CNL7), Northern epilepsy neuronal ceroid lipofuscinosis (CNL8), Turkish late infantile neuronal ceroid lipofuscinosis (CNL8), German/Serbian late infantile neuronal ceroid lipofuscinosis (CNL9), Congential cathepsin D deficiency neuronal ceroid lipofuscinosis (CTSD), Cystinosis, Farber disease, Fucosidosis, Galactosialidosis, Gaucher disease types 1, 2 and 3, GM1 gangliosidosis, GM2 gangliosidosis (e.g., AB variant), Hunter disease, Hurler-Scheie disease, Hurler syndrome, hyaluronidase deficiency, Scheie syndrome, Krabbe disease, alpha-Mannosidosis, beta-Mannosidosis, Maroteaux-Lamy syndrome, Metachromatic leukodystrophy, Multiple sulfatase deficiency, mucolipidin 1 deficiency, Morquio A disease, Morquio B disease, Mucolipidosis II/III, Niemann-Pick type A disease, Niemann-Pick type B disease, Niemann-Pick type C disease, Niemann-Pick type D disease, Pompe disease, pseudo-Hurler polydystrophy/phosphotransferase deficiency, Lysosomal acid lipase deficiency, Sandhoff disease, Sanfilippo A syndrome, Sanfilippo B syndrome, Sanfilippo C syndrome, Sanfilippo D syndrome, Schindler disease, Schindler-Kanzaki disease, Sialidosis, Sly syndrome, Danon disease, Cholesteryl ester storage disease, Tay-Sachs disease, Pycnodysostosis, Salla disease, infantile free sialic acid storage disease and Wolman disease. Preferably, the lysosomal storage disease is preferably Niemann-Pick type C disease.

The term Niemann-Pick disease type C or abbreviated as “NPC,” refers to the disorder as it is known in the art, and is distinct from Type A or B. NPC is a rare and fatal, autosomal recessive, neurodegenerative disease that can present in infants, children, or adults. Its incidence in Western Europe is 1/90,000. Approximately 95% of patients with NPC have mutations in NPC1, a gene implicated in intracellular cholesterol trafficking. Mutation of NPC1 causes intracellular accumulation of unesterified cholesterol in late endosomal/lysosomal structures and marked accumulation of glycosphingolipids, especially in neuronal tissue but also in liver, spleen and lungs. Thus, NPC patients generally present with hepatosplenomegaly (enlargement of liver and spleen) and neurological degeneration.

A prenatal syndrome of nonimmune fetal hydrops can be the first symptom of NPC disease. Neonates can present with severe liver disease from infiltration of the liver and/or respiratory failure. Alternatively, infants without liver or pulmonary disease have hypotonia and developmental delay. The classic presentation occurs in mid-to-late childhood with onset of ataxia, vertical supranuclear gaze palsy (VSGP), and dementia. Regression is common and seizures are frequent. Neurological symptoms can become disabling, making oral feeding impossible; death usually occurs in the late second or third decade from aspiration pneumonia. Adults can be more mildly affected and are more likely to present with dementia or psychiatric symptoms. There are no proven treatments for NPC, and after the diagnosis, fatal neurodegeneration is inevitable.

The diagnosis of NPC disease is confirmed by specialized biochemical testing that demonstrates cholesterol storage which can be detected by filipin staining in cultured fibroblasts or other methods known in the art. Most individuals with NPC disease have NPC type 1, caused by mutations in NPC1; fewer than 20 individuals have been diagnosed with NPC type 2, caused by mutations in NPC2. Molecular genetic testing of NPC1 and NPC2 detects disease-causing mutations in approximately 94% of individuals with NPC disease, almost all of whom have mutations in NPC1. NPC disease, regardless of the locus and allele(s), is a recessive metabolic condition and the mutations are loss of function or reduced function. Therefore, providing and expressing a single copy of the wild type gene can completely restore NPC1 or 2 enzymatic function.

Additionally, any other disease or condition known in the art to be susceptible to treatment with a mRNA of the invention including a mRNA encoding NPC intracellular cholesterol transporter 1 (NPC-1) and NPC intracellular cholesterol transporter 2 (NPC-2) may be contemplated for treatment with the mRNA of the invention. In this case, the subject may be known to have, or suspected of having a mutation to either gene on one or both loci of either or both of NPC-1 and NPC-2. In certain embodiments, the subject has a mutation to NPC-1 selected from the group consisting of Gly248Val, Met1142Thr, Ile1 061 Thr and Arg404Trp. In this embodiment, treatment of a subject with a mRNA of the invention improves one or more symptoms associated with the mutation.

A subject in need of treatment may present a number of symptoms depending on the type of disorder that is present. In an aspect, a subject in need of treatment may exhibit well characterised symptoms associated with a disease or condition described herein.

A subject may be identified as having a lysosomal storage disease by presenting to a clinician with symptoms of a lysosomal storage disease, including but not limited to an enlarged liver and spleen. Storage may begin during early embryonic development, and the clinical presentation for lysosomal storage diseases can vary from an early and severe phenotype to late-onset mild disease. Said subject may be subject to a variety of diagnostic tests to determine if the subject has a lysosomal storage diease, and further to determine the presence of non-cholesterol lipids and macromolecules and non-cholesterol dominant lipids (i.e., non-cholesterol lipids that are present in an amount or percentage greater than that of cholesterol).

For example, ultrastructural examinations of skin biopsy specimens can be used to detect lysosomal accumulation of undegraded metabolites. A test of specific lysosomal enzyme activity can also be used to determine the presence of specific lysosomal enzymes. Moreover, correlation of both skin ultrastructure and assay for specific lysosomal enzymes in cultured dermal fibroblasts derived from the skin biopsy may also facilitate determination of cholesterol and non-cholesterol lipids, and diagnostic accuracy. Filipin staining is a well-known histochemical stain for cholesterol. Filipin is highly fluorescent and binds specifically to cholesterol. This method of detecting cholesterol in cell membranes is used clinically, for example in the study and diagnosis of NPC disease. Molecular genetic testing can be used, may be use to refine the enzymatic diagnosis. Other diagnostic methods to determine the present of cholesterol and non-cholesterol lipids include antibody immunostaining or mass spectrometry.

By way of another example, a subject with Niemann-Pick type C disease may exhibit one or more of the following symptoms:
enlarged liver and spleen (hepatosplenomegaly)
difficulty coordinating movement (ataxia)
abnormal eye movements (vertical supranuclear gaze palsy)
poor muscle tone (hypotonia)
severe liver disease
frequent respiratory infections
difficulty with speech
difficulty with swallowing and feeding
loss of cognitive skills
seizures.

It is envisaged that the current invention encompasses the identification of a subject suffering from NPC disease using diagnostic tests known in the art to assess symptoms listed above. Alternatively, a subject may have an identified mutation in a NPC1 or NPC2, therefore being likely to develop NPC disease. Subjects exhibiting one or more of these symptoms are suitable for treatment with a mRNA of the invention.

The terms “treatment” or “treating” of a subject includes the administration of a mRNA or pharmaceutical composition of the invention to a subject with the purpose of delaying, slowing, stabilizing, curing, healing, alleviating, relieving, altering, remedying, less worsening, ameliorating, improving, or affecting the condition or disease, or a symptom of the disease or condition including those listed above.

The term “treating” refers to any indication of success in the treatment or amelioration of an injury, pathology or condition, including any objective or subjective parameter such as abatement; remission; lessening of the rate of worsening; lessening severity of the disease; stabilization, diminishing of symptoms or making the injury. In particular, treating refers to a reduction in a symptoms associated with the disease or condition.

The existence of, improvement in or treatment of the disease or condition may be determined by any clinically or biochemically relevant method known in the art. A relevant method may be measurement of symptoms. It is generally understood that the expression of a protein encoded by a mRNA of the invention will provide for an improvement of one or more of the following symptoms of a lysosomal storage disease. For example in the case of Niemann-Pick type C disease, an improvement in symptoms may include one or more of:
reduced liver and spleen hepatosplenomegaly
improvement in coordination (improved ataxia)
improved eye movements (improved vertical supranuclear gaze palsy)
improved muscle tone (improved hypotonia)
reduced liver disease
reduced respiratory infections
improved speech
reduced difficulty with swallowing and feeding
improved cognitive skills
reduced seizures.

The improvement may be determined directly from the subject, or a sample or biopsy therefrom. The sample or biopsy may be of the diseased tissue.

In addition to treatment with an mRNA of the invention, the current invention also contemplates additional treatments which may be administered together or separately. Such treatments may include administering an inhibitor of glucosylceramide synthase, an inducer of heat shock proteins, a cyclic oligosaccharide or hydroxypropyl-beta-cyclodextrin. Treatments such as these, or others known by a skilled person have been shown to have varying degrees of effectiveness in the treatment of NPC disease.

It will be understood that by determining one or more of restored levels of protein, increased protein expression, increased function or increased cholesterol esterification, a mRNA of the invention will in some embodiments, be sufficient for the effective treatment of a lysosomal storage disorder described herein, preferably Niemann-Pick type C disease.

### Kits

In an aspect, the present invention provides a kit comprising one or more of the following:
(i) a mRNA of the invention;
(ii) a pharmaceutical composition of the invention.

In the case of a kit for therapeutic use, the kit can additionally comprise a pharmaceutically acceptable carrier, diluent or excipient.

Optionally, a kit of the invention is packaged with instructions for use in a method described herein according to any example.

### EXAMPLE

### Material and Methods

### Cell Culture

HeLa, U87 MG and Hep G2 cells (ATCC) were cultured in Dulbecco's Modified Eagle's Medium (DMEM; Lonza) supplemented with 10% fetal bovine serum (FBS; Bovogen), 100 U/ml penicillin and 100 μg/ml streptomycin (Thermo Fisher Scientific). Healthy human control fibroblasts (GM03652G) and NPC1 patient fibroblasts (GM18393 and GM17919A) were purchased from the Coriell Cell Repository (Table 1) and cultured in DMEM (Lonza) supplemented with 20% FBS (Bovogen), 100 U/ml penicillin and 100 μg/ml streptomycin (Thermo Fisher Scientific). For cholesterol esterification assays, fibroblasts were switched to DMEM supplemented with 5% fetal bovine lipoprotein deficient serum (Alpha Diagnostic International), 100 U/ml penicillin and 100 μg/ml streptomycin. All cell lines were incubated at 37° C. with 5% CO₂.

TABLE 1
Characterization of primary fibroblast cell lines used in this study

| Cell line | Identified mutation (allele 1) | Identified mutation (allele 2) |
|---|---|---|
| GM03652 male | None (wildtype) | None (wildtype) |
| fibroblast | | |
| GM18393 male | GLY248VAL (chromosomal | MET1142THR (chromosomal |
| fibroblast | location 18q11-q12) | location 18q11-q12) |
| GM17919 female | ILE1061THR (chromosomal | ARG404TRP (chromosomal |
| fibroblast | location 18q11-q12) | location 18q11-q12) |

mRNA Sequence Engineering

Codon optimization was implemented to enhance the translatability and structural stability of synthesized mRNAs. The GeneArt-optimized luciferase coding sequence was obtained by inputting the wildtype sequence into the online GeneOptimizer portal (Thermo Fisher Scientific). The GC3-optimized luciferase coding sequence was obtained by substituting, wherever possible, wildtype codons with codons that had a G or C at the third base position. All NPC1 mRNA variants were also codon optimized in the same way (GC3 optimization). This strategy is based on evidence that such codon bias confers enhanced stability and translatability to mRNAs in humans (Kariko et al (2004)). Detailed codon selection parameters are listed in Table 2. Initial codon optimization was performed by selecting codons with a G or C at the third base position. Where two or more GC3-optimal codons existed, the inventors performed a secondary selection on the basis of which codon had the highest human codon usage frequency. For certain genes, GC3 codon optimization renders the gene too complex for synthesis via commercial gene synthesis technologies. Thus, the GC3 codon selection parameters sometimes need to be relaxed to allow usage of GC3-nonoptimal codons, such that genes can be fully synthesized.

All mRNAs harbored an EEF1B2-001 5′ UTR upstream of the open reading frame, and an alpha-1 globin 3′ UTR and 100-nt poly(A) tail downstream of the open reading frame. These sequences have been shown to further enhance the stability and translatability of IVT mRNAs across multiple cell types (Floor and Doudna (2016); Wang et al (1995)).

TABLE 2
Codon selection parameters used for GC3 codon
optimization in this study

| Amino acid | GC3-optimal codon |
|---|---|
| Alanine (A) | GCC |
| Cysteine (C) | TGC |
| Aspartic acid (D) | GAC |
| Glutamic acid (E) | GAG |
| Phenylalanine (F) | TTC |
| Glycine (G) | GGC |
| Histidine (H) | CAC |
| Isoleucine (I) | ATC |
| Lysine (K) | AAG |
| Leucine (L) | CTG |
| Methionine (M) | ATG |
| Asparagine (N) | AAC |
| Proline (P) | CCC |
| Glutamine (Q) | CAG |
| Arginine (R) | CGG |
| Serine (S) | AGC |
| Threonine (T) | ACC |
| Valine (V) | GTG |
| Tryptophan (W) | TGG |
| Tyrosine (Y) | TAC |

### Generation of DNA Templates for In Vitro Transcription

Wildtype, GeneArt and GC3 luciferase variants were ordered as separate GenParts DNA fragments (Genscript). The GC3-optimized NPC1 sequence was encoded in a plasmid (Twist Bioscience) and linearized via double restriction enzyme digestion using BamHI-HF (NEB) and HindIII-HF (NEB). Successful linearization was confirmed via 1% agarose gel electrophoresis. Templates for IVT were generated by performing high-fidelity PCR using Q5 Hot Start High-Fidelity DNA Polymerase (NEB) and primers 1 and 2 (IDT; sequences provided in Table 3). From 5′ to 3′, each double-stranded DNA template contained a T7 promoter, a 5′ UTR, open reading frame, 3′ UTR, and 100-nt poly(A) sequence (introduced via primer 2). After linearization and PCR amplification steps, DNA products were purified using the DNA Clean & Concentrator-5 Kit (Zymo Research). All purified PCR products were sequence verified via 1% agarose gel electrophoresis and assessed for purity via Nanodrop (Thermo Fisher Scientific).

TABLE 3
Primers used in this study for generation of DNA templates

| Primer # | Sequence (5′ to 3′) |
|---|---|
| 1 | CCACGAATCTAATACGACTCACTATAAGG (SEQ ID NO: 12) |
| 2 | TTTTTTTTTTTTTTTTTTTTTTTTTTTTTTTTTTTTTTTTTTTTTTTT |
| | TTTTTTTTTTTTTTTTTTTTTTTTTTTTTTTTTTTTTTTTTTTTTTTT |
| | TTTTGCCGCCCACTCAGACTTTATTC (SEQ ID NO: 13) |

mRNA Synthesis and Purification

mRNAs were transcribed from DNA templates using T7 RNA polymerase (HiScribe T7 High Yield RNA Synthesis Kit, NEB). To generate base modified mRNA variants, where specified uridine 5′ triphosphate (UTP) was completely replaced with either N1-methylpseudouridine 5′-triphosphate (M1ψTP, APExBIO) or 5-methoxyuridine 5′-triphosphate (5moUTP, APExBIO), and cytidine 5′-triphosphate (CTP) was completely replaced with 5-methylcytidine 5′-triphosphate (5mCTP, APExBIO). Co-transcriptional capping was performed using the EZ Cap Reagent AG (3′ OMe) (APExBIO), resulting in a stable Cap1 structure at the 5′ end of synthesized mRNA transcripts. After IVT reactions had run to completion, DNA templates were digested using DNase I (NEB). mRNAs were subsequently purified using a protocol that has been described previously (FIG. 1B) (Baiersdörfer et al (2019)). Briefly, transcripts were isolated by precipitation with 8 M LiCl (Sigma-Aldrich), dissolved in nuclease-free water, and incubated in chromatography buffer containing 10 mM HEPES (pH 7.2), 0.1 mM EDTA, 125 mM NaCl, and 16% (v/v) ethanol (prior to adding mRNA, RNAsecure RNase Inactivation Reagent (Thermo Fisher Scientific) was added to the chromatography buffer and the buffer was heated to 60° C. for 10 minutes to eliminate any potential RNases in the buffer; the buffer was allowed to cool before adding the mRNA).

The mRNA-containing buffer was added to a spin column containing prewashed cellulose fibers (Sigma-Aldrich) and incubated at room temperature for 30 minutes under vigorous shaking. The mixing process promotes the association of cellulose with double-stranded RNA (dsRNA) contaminants in solution that are an unwanted by-product of the IVT process and cannot be eliminated using standard mRNA purification processes. After 30 minutes, the spin column was centrifuged at 14,000×g for 60 seconds and the flowthrough containing single-stranded mRNA was transferred to a new spin column containing prewashed cellulose. The 30-minute incubation procedure was then repeated, followed by centrifugation. mRNA was isolated from the flowthrough by adding 0.1 volume 3 M sodium acetate, pH 5.2 (Thermo Fisher Scientific) and 1 volume isopropanol (Sigma-Aldrich), followed by incubation at room temperature for 15 minutes and centrifugation at 16,000×g for 15 minutes. The precipitated mRNA was dissolved in 1 mM sodium citrate, pH 6.5 (THE RNA Storage Solution, Thermo Fisher Scientific). mRNA integrity was assessed via gel electrophoresis by running denatured aliquots on a non-denaturing 1% agarose gel, using SYBR Safe DNA Gel Stain (Thermo Fisher Scientific) to visualize bands. mRNA concentration and purity were assessed via Nanodrop (Thermo Fisher Scientific).

### Luciferase Assay

Cells were seeded at varying densities in white, flat bottom 96-well plates (Corning). The next day, mRNA was complexed with Lipofectamine MessengerMax (Thermo Fisher Scientific) in Opti-MEM I Reduced Serum Medium (Thermo Fisher Scientific) according to the manufacturer's instructions, using 2 μl Lipofectamine reagent per μg of mRNA. Complexed mRNA was added to cells in serum-containing culture medium. At various timepoints post-transfection (6, 24 or 48 hours), plates were retrieved from the incubator and allowed to equilibrate to room temperature, before reconstituted luciferase reagent (ONE-Glo EX, Promega) was added to cells in culture medium, following the manufacturer's protocol. Plates were incubated at room temperature for 2-3 minutes with moderate shaking to facilitate cell lysis and sample equilibration. Luminescence was subsequently measured using an Infinite M200 Pro plate reader (Tecan).

In Silico Prediction of mRNA Secondary Structures

mRNA secondary structures were predicted by inputting mRNA sequences into the RNAfold webserver provided by the Vienna RNA Websuite Lorenz et al (2011)). RNAfold computes the minimum free energy (MFE) and optimal secondary structure for a given sequence using experimentally determined thermodynamic parameters. Default RNA parameters from the 2004 Turner model were used to generate secondary structure predictions (Mathews et al (2004)).

Determination of mRNA Melting Curves Via Differential Scanning Fluorimetry

mRNA thermal stability was assessed via differential scanning fluorimetry according to a previously published protocol (Silvers et al (2015)). Briefly, 1 μg aliquots of mRNA were mixed with Quant-iT RiboGreen RNA reagent (Thermo Fisher Scientific) and brought to a final volume of 50 μl with nuclease-free water, such that the final RiboGreen concentration was 1×. Samples were added to separate wells of a 48-well PCR plate (Bio-Rad) and incubated in a MiniOpticon Real-Time PCR System (Bio-Rad). A melting curve routine was initiated, whereby samples were incubated at 20° C. for 3 minutes, followed by heating at a constant rate from 20° C. to 90° C. Fluorescence intensities were recorded at each incremental temperature using the excitation and emission settings for SYBR Green (since RiboGreen and SYBR Green have very similar spectral properties). Once the temperature reached 90° C., samples were cooled to 20° C. and allowed to refold for 15 minutes, at which point the melting curve routine was repeated. Melting curve experiments were performed 3 times, with all samples run in duplicate. The negative first derivative of fluorescence intensities were then analyzed as a function of temperature.

### NPC1 Immunofluorescence Staining

Cells were seeded at varying densities in black, clear-bottom CellCarrier-96 Ultra microplates (PerkinElmer). The next day, cells were transfected with NPC1 mRNA using Lipofectamine MessengerMax reagent, as described above. 24 hours after transfection, cells were fixed in 4% paraformaldehyde for 20 minutes at room temperature, washed with PBS, and permeabilized for 1 hour with 0.2% saponin (Sigma-Aldrich) diluted in PBS containing 10% normal goat serum (Thermo Fisher Scientific). Cells were then incubated for 1 hour at room temperature with anti-NPC1 primary antibody (ab134113, Abcam) diluted 1:200 in PBS containing 0.2% saponin and 10% normal goat serum. Cells were subsequently washed with PBS containing 0.1% Tween 80, and incubated for 1 hour with Alexa Fluor 488-labelled goat anti-rabbit secondary antibody (Thermo Fisher Scientific) diluted 1:500 in PBS containing 0.2% saponin and 10% normal goat serum. After washing with 0.1% Tween 80, cells were stained 1:10000 with Hoescht 33342 (Thermo Fisher Scientific) for 10 minutes, washed once with PBS, and stored in PBS at 4° C. until required for imaging. Cells were imaged using a PerkinElmer Operetta CLS High Content Imaging System with a 20× high numerical aperture dry objective lens and AF488 and DAPI filter sets. At least 7 imaging fields were analyzed per well, and every experimental condition was run in triplicate, equating to an analysis of >300 cells per treatment condition.

### Amplex Red Unesterified Cholesterol Assay

Cells were seeded at a density of 3000-5000 cells/well in CellCarrier-96 Ultra microplates. After 24 hours, cells were transfected with NPC1 mRNA using Lipofectamine MessengerMax reagent, as described above. 24 or 48 hours after transfection, the cell culture medium was removed and cells were washed in PBS twice, before 100 μl reconstituted Amplex Red cholesterol assay reagent (Thermo Fisher Scientific), excluding cholesterol esterase, was added to each well as per the manufacturer's instructions. After incubation at 37° C. for 1 hour, the fluorescence intensity in each well was measured (Ex=560 nm; Em=590 nm) using an Infinite M200 Pro plate reader.

alamarBlue Cell Viability Assay

mRNA transfections were carried out in 96-well plates, as described above. 24 or 48 hours after transfection, alamarBlue cell viability reagent (Thermo Fisher Scientific) was added to the cell culture medium in a 1:10 ratio (alamarBlue: medium), as per the manufacturer's instructions. Cells were incubated at 37° C. in the dark for between 1-4 hours, and then the fluorescence intensity in each well was measured (Ex=560 nm; Em=590 nm) using an Infinite M200 Pro plate reader.

### Cholesterol Esterification Assay

Cells were seeded in CellCarrier-96 Ultra microplates at a density of 3000-5000 cells/well in DMEM containing 20% FBS. The next day, cells were switched to DMEM containing 5% fetal bovine lipoprotein deficient serum. After 24 hours, cells were transfected with NPC1 mRNA using Lipofectamine MessengerMax reagent, as described above. 24 hours after transfection, the culture medium was replaced with either Opti-MEM or Opti-MEM spiked with 50 μg/ml LDL. After a further 6 hours, intracellular levels of unesterified cholesterol and total cholesterol were separately assayed using the Amplex Red Cholesterol Assay Kit (Promega), following the manufacturer's instructions. Levels of esterified cholesterol were calculated by subtracting the amount of esterified cholesterol from the total cholesterol amount. The addition of LDL after 48 hours of sterol depletion provides a source of cholesterol that can be used by cells for esterification. While healthy cells are expected to demonstrate a burst of cholesterol esterification in response to this, NPC patient cells tend to show low levels of esterification regardless of how much cholesterol is available.

### Filipin Staining

The same protocol was followed as in the cholesterol esterification assay above. After the 6-hour incubation with 50 μg/ml LDL or Opti-MEM alone, cells were fixed in 4% paraformaldehyde for 20 minutes at room temperature and then washed with PBS. Cells were subsequently stained with 50 μg/ml filipin (Sigma-Aldrich; freshly dissolved in DMSO at 10 mg/ml and then diluted in PBS) in the dark at room temperature for 1 hour. After washing, cells were stored in PBS at 4° C., and later imaged using a PerkinElmer Operetta with a 20× high numerical aperture dry objective lens and the DAPI filter set. At least 9 imaging fields were analyzed per well, and every experimental condition was run in triplicate, equating to an analysis of >600 cells per treatment condition.

### Lysotracker Red Staining

The same protocol was followed as in the cholesterol esterification assay above. After the 6-hour incubation with 50 μg/ml LDL or Opti-MEM alone, cells were live-stained with 50 nM Lysotracker Red DND-99 (Thermo Fisher Scientific) in Opti-MEM at 37° C. for 1 hour. Cells were subsequently washed with PBS, fixed in 4% paraformaldehyde, and stained with 1:10,000 Hoechst 33342, before being stored in PBS at 4° C. Imaging was performed using a PerkinElmer Operetta with a 40× high numerical aperture dry objective lens and TRITC and DAPI filter sets. At least 9 imaging fields were analyzed per well, and every experimental condition was run in duplicate, equating to an analysis of >300 cells per treatment condition.

### Statistics

Experimental data were analyzed used Prism 9 Software (GraphPad), by either ordinary one-way ANOVA with a Dunnett's multiple comparisons test, or two-way ANOVA with a Tukey's multiple comparisons test.

### Results

“GC3” Codon Optimization Enhances mRNA Translation, in Part Due to Increased mRNA Structural Stability

An mRNA molecule's competence for protein translation is governed by two factors: functional half-life and translational efficiency (Mauger et al (2019)). The former is defined by the length of time within which an mRNA molecule is capable of generating protein, while the latter is defined by the mRNA's ability to recruit ribosomes to initiate translation. The coding sequence plays an important role in shaping both of these factors, and thus governs an mRNA's translatability to a considerable extent. Codon optimization, the process of replacing wildtype codons with more “optimal” synonymous codons, is a well-known and widely used strategy for enhancing the expression of protein-coding genes (Mauro and Chappell (2014)). However, specific strategies for codon optimization vary considerably based on different underlying assumptions about which codon features are important for translation. One approach involves substituting in the most frequently used codon for all instances of a given amino acid (Villalobos et al (2006)). Another approach involves only replacing rare codons with more abundant synonymous codons (Deng et al (1997)). Still other approaches involve adjusting the codon usage frequency to match the natural frequencies in a host organism (Angov et al (2008); Wu et al (2006)), or choosing codons based on cognate transfer RNA (tRNA) abundance (Qian et al (2012)). Many available codon optimization algorithms involve a weighted combination of two or more different optimization parameters (Villalobos et al (2006); Wu et al (2006); Raab et al (2010)).

A recent study by Hia et al. revealed that human mRNAs comprising codons with G or C at the third base position (GC3) are associated with increased stability relative to mRNAs harboring codons with either A or U at the third base position (Hia et al (2019)). They proposed that GC3 codons contribute to mRNA stability in part by preventing interactions with RNA-binding proteins that typically act to induce transcript decay (such as ILF2 and ILF3) (Hia et al (2019)). In light of this finding, as well as other corroborating reports (Thess et al (2015); Vaidyanathan et al (2018)), the inventors sought to explore whether GC3 optimization is a viable strategy for enhancing heterologous mRNA expression in vitro. To this end, the inventors engineered a firefly luciferase (Luc) coding sequence based on the GC3 optimization strategy, globally optimizing every amino acid in the sequence. Where 2 or more synonymous GC3 codons existed, the inventors made a selection on the basis of which codon had the highest human codon usage frequency. The inventors sought to compare the expression profile of GC3-optimized Luc to wildtype Luc, as well as a Luc variant optimized by a different codon optimization strategy (for the purpose of this study, the inventors used the GeneOptimizer algorithm provided by Thermo Fisher via its GeneArt gene synthesis service) (Raab et al (2010)). The inventors designed all 3 mRNAs to harbor a Cap1 structure and EEF1B2-001 5′ UTR immediately upstream of the coding sequence, and an alpha-1 globin 3′ UTR and 100-nt poly(A) tail downstream. Both the EEF1B2-001 5′ UTR and alpha-1 globin 3′ UTR were chosen based on previous reports that showed their inclusion significantly enhanced mRNA stability and expression in vitro (Floor et al (2016); Wang et al (1995)).

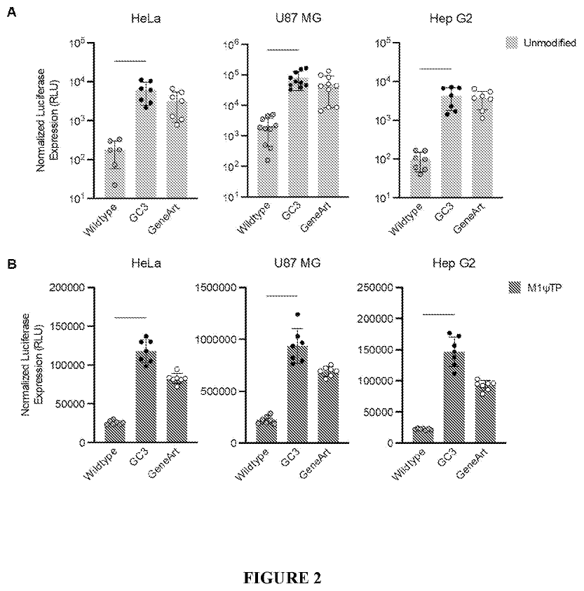

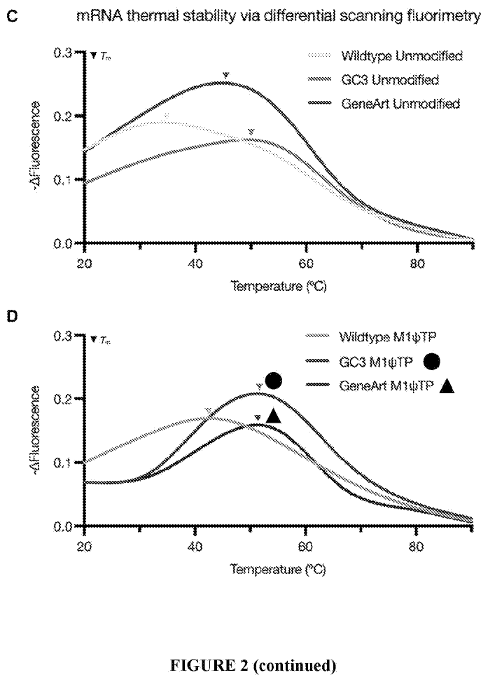

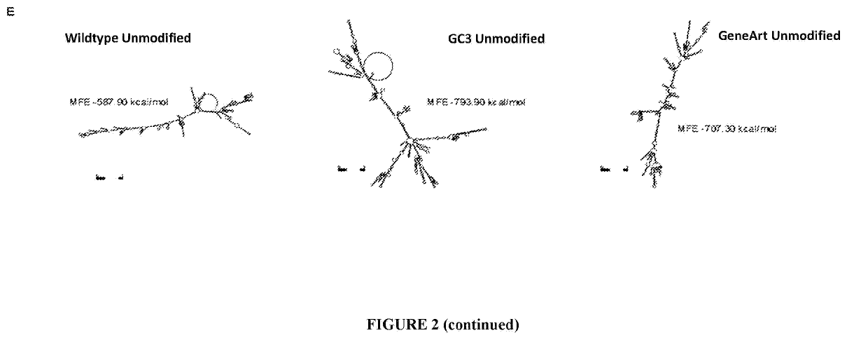

The inventors initially produced the 3 Luc mRNAs using all unmodified nucleotides, following a uniform synthesis and purification protocol (FIG. 1B). After transfecting 3 different cell lines, the inventors observed that both codon-optimized Luc variants consistently outperformed wildtype Luc by more than an order of magnitude (FIG. 2A). However, the inventors observed no meaningful difference in expression profiles between the 2 codon optimization strategies. Given that base modification has been shown to enhance mRNA translation while reducing immune activation (Svitkin et al (2017); Andries et al (2015)) the inventors next investigated the expression profiles of the 3 Luc variants when uridine triphosphate (UTP) was globally replaced with N1-methylpseudouridine triphosphate (M1ψTP). Again, the inventors observed that both codon-optimized variants outperformed wildtype Luc across the 3 different cell lines, while all three variants outperformed their unmodified counterparts (FIG. 2B). This time, however, the inventors observed that GC3-optimized Luc produced meaningfully greater protein expression than GeneArt-optimized mRNA.

Having confirmed that GC3 optimization is a viable strategy for enhancing mRNA translation in vitro, the inventors next sought to investigate why this might be the case. A previous report by Mauger et al. showed that increased mRNA secondary structure leads to enhanced protein expression via an increase in mRNA functional half-life (Mauger et al (2019)). Since an mRNA's GC content tends to be positively correlated with its degree of secondary structure, the inventors probed whether GC3 optimization enhances the secondary structure of Luc mRNA. The inventors used differential scanning fluorimetry, a validated technique that uses an RNA-specific fluorescent reporter dye to measure the structural stability of RNA constructs in the presence of altered environmental parameters such as temperature, salts or pH (Silvers et al (2015)). The inventors generated melt curves for the different mRNA variants, running each variant through three consecutive cycles of gradual heating from 20° C. to 90° C. followed by gradual cooling back to 20° C. Using data from the second heating/cooling cycle (due to the greater reliability of the data across several experimental runs), the inventors determined the peak melting temperature (Tm) for each variant. Consistent with the protein expression data, the inventors observed that both GeneArt unmodified Luc and GC3 unmodified Luc underwent melting at considerably higher temperatures (˜46° C. and ˜51° C.) than unmodified wildtype Luc (˜35° C.), indicating greater mRNA structural stability (FIG. 2C). This trend also held for M1ψTP modified constructs, although base modification appeared to dramatically increase the Tₘ of wildtype Luc (˜35° C. to ˜43° C.) while inducing a much less significant increase for codon-optimized mRNAs (FIG. 2D). An estimation of the minimum free energy (MFE) and optimal secondary structure of the unmodified Luc variants using the RNAfold webserver (Lorenz et al (2011)) further corroborated these findings (FIG. 2E), with wildtype Luc estimated to have a considerably higher MFE than GeneArt and GC3 Luc (a lower MFE indicates greater structural stability). Together, these data suggest that codon optimization—and in particular GC3 optimization—results in increased mRNA stability due to an increase in secondary structure, and that this may play an important role in enhancing protein expression. The possible mechanism(s) by which mRNA secondary structure enhances translation have been discussed elsewhere (Mauger et al (2019)).

GC3 Codon Optimization and N1-Methylpseudouridine Base Substitution Synergistically Enhance mRNA Translation

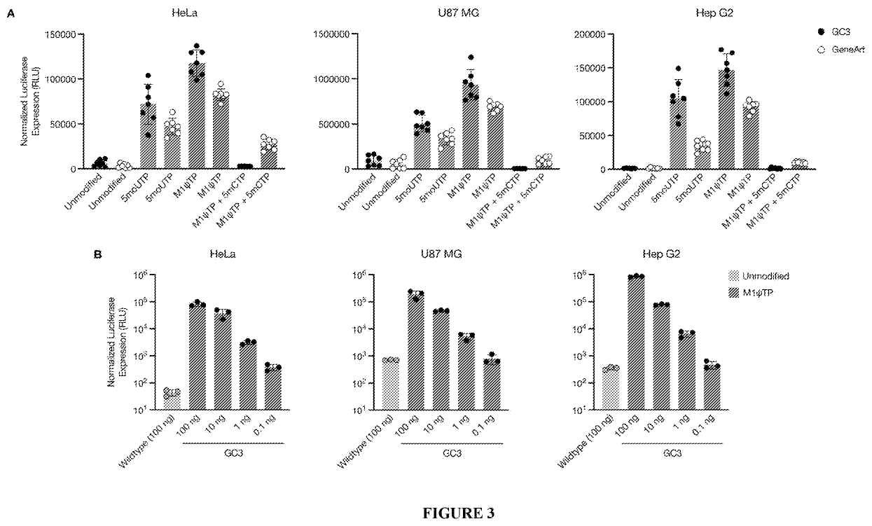

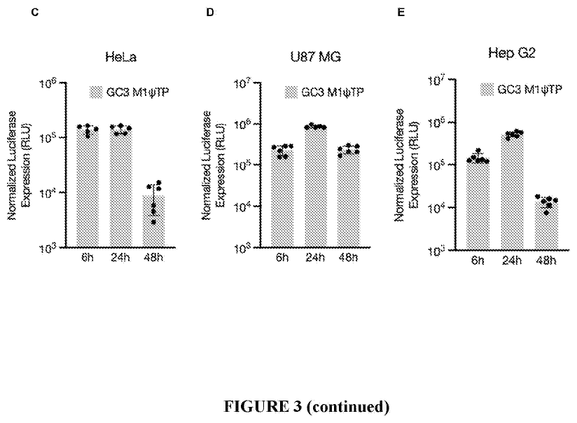

Next, the inventors investigated the impact of different mRNA base modification strategies on protein expression in vitro. M1ψTP and 5-methoxyuridine triphosphate (5moUTP) are among the most commonly used UTP substitutes, while 5-methylcytidine triphosphate (5mCTP) is sometimes used in place of cytidine triphosphate (CTP) (Li et al (2016)). Thus, the inventors engineered GC3 and GeneArt Luc mRNAs with 4 different base compositions: (1) all unmodified bases, (2) 5moUTP in place of UTP, (3) M1ψTP in place of UTP, and (4) M1ψTP in place of UTP and 5mCTP in place of CTP. The inventors transfected HeLa, U87 MG and Hep G2 cells with these 8 mRNA variants and assayed Luc expression at 24 h. All 3 cell lines exhibited relatively uniform expression profiles, with the different base modification strategies ranking, in order of Luc expression from highest to lowest, M1ψTP>5moUTP>M1ψTP+5mCTP>unmodified (FIG. 3A). The inventors observed that the combination of GC3 codon optimization with M1ψTP base modification cumulatively resulted in the highest protein expression across all cell lines. M1ψTP has been shown to enhance mRNA translation by increasing ribosome recruitment and thus improving translational efficiency (Svitkin et al (2015)). Time course experiments revealed that kinetic Luc expression profiles varied across the three cell lines, with expression peaking at 6 h in HeLa cells but at 24 h in U87 MG and Hep G2 cells (FIG. 3C-D). Interestingly, although GC3 optimization outperformed GeneArt optimization across both 5moUTP and M1ψTP modifications, the inverse was true for the 5mCTP base modification strategy. The dramatic reduction in expression when 5mCTP was combined with M1ψTP, relative to M1ψTP alone, has previously been observed in cells and cell-free extracts (Svitkin et al (2015)). Thus, the inventors conclude that coupling GC3 optimization with M1ψTP base modification leads to synergistic enhancement of mRNA translation in vitro. In the inventors hands, this combination generated an mRNA that was approximately 1000-fold more potent than wildtype, unmodified Luc mRNA (FIG. 3B).

Engineered NPC1 mRNA Restores Functional Protein Expression and Reverses Disease Pathology in NPC1 Patient Fibroblasts

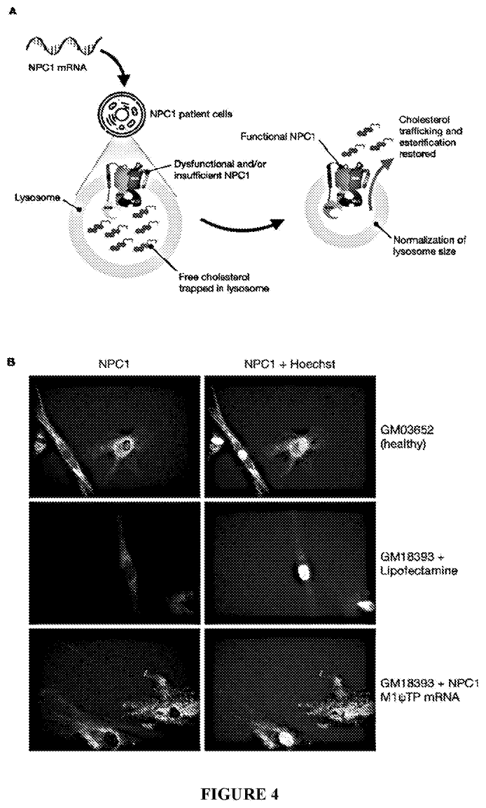

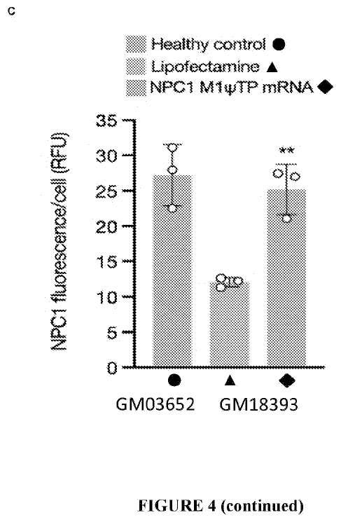

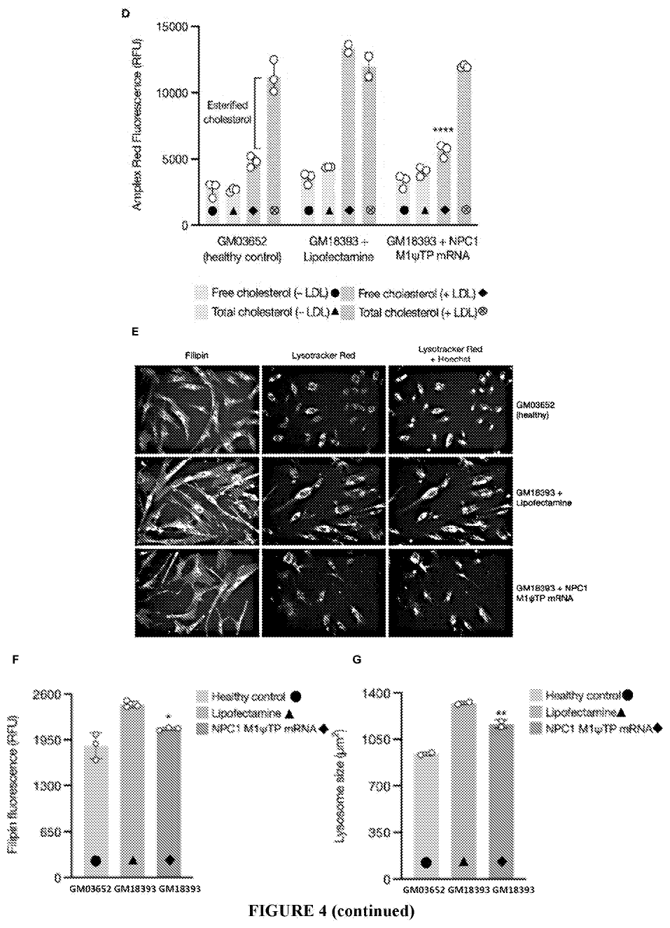

Having successfully engineered a highly expressing Luc mRNA, the inventors next sought to apply their findings to the engineering of NPC1 mRNA (FIG. 4A). The inventors performed GC3 optimization of the NPC1 coding sequence and appended the same 5′ and 3′ flanking regions as before to enable efficient protein expression. The inventors synthesized 3 NPC1 mRNA variants, all comprising identical sequences but utilizing different base compositions (unmodified, M1ψTP-modified, and 5moUTP-modified), to test whether the same trend that the inventors observed in reporter assays extended to a human in vitro disease model. The inventors did not synthesize constructs with 5mCTP due to the dramatic decrease in overall expression that the inventors observed in Luc reporter assays when 5mCTP base modification was combined with M1ψTP modification. To determine whether the NPC1 mRNA constructs could induce functional protein expression in vitro, the inventors transfected them into human fibroblasts derived from two clinically affected NPC1 patients (GM18393 and GM17919), both of whom were previously shown to harbor compound heterozygous mutations at the NPC1 gene locus, with different exons affected for each donor. All 3 NPC1 mRNA variants generated significantly increased protein expression in both cell lines compared to the Lipofectamine-treated control, as measured by high-content confocal imaging (FIGS. 4B, 5A and 5B). Having established that all 3 mRNA variants could induce significant NPC1 protein expression in vitro, the inventors performed all subsequent experiments using NPC1 M1ψTP mRNA alone. Additionally, due to an insufficiency of GM1 7919 cells, the inventors performed all further experiments with only GM18393 cells. Quantification of NPC1 protein expression revealed that treatment with NPC1 M1ψTP mRNA boosted NPC1 protein levels to the same as those observed in healthy, wildtype donor fibroblasts (FIG. 4C).

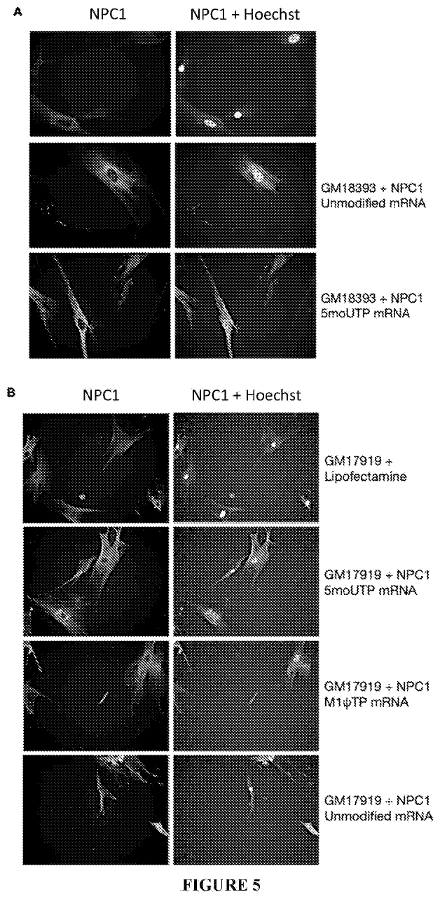

Next, the inventors evaluated whether the mRNA-induced increase in NPC1 protein levels corresponded to phenotypic rescue. Assaying the levels of free, unesterified cholesterol in treated and untreated cells confirmed that the expressed NPC1 protein was functional, since transfected cells showed a clear, albeit modest, reduction in free cholesterol levels at 48 h post-transfection (FIG. 6A). Moreover, an mRNA dose of 50 ng yielded a phenotypic improvement (a reduction in filipin levels), whilst the highest mRNA dose (200 ng) produced an increase in filipin staining well above the Lipofectamine-treated control (FIG. 6B). Surprisingly, the inventors observed an inverse dose-response relationship, where a lower mRNA dose (25 ng) corresponded to phenotypic improvement (reduced free cholesterol) whereas a higher mRNA dose (100 ng) produced no such improvement. To further probe the downstream phenotypic consequences of mRNA treatment, the inventors performed a cholesterol esterification assay, which is commonly used to diagnose NPC (Pentchev et al (1986); Park et al (2003)). Cells were seeded in full serum media, which was subsequently replaced with media containing lipoprotein-deficient serum the following day; a 48 h incubation in lipoprotein-deficient serum served to deplete cells of cholesterol, after which 50 μg/ml LDL was added directly to the cells and incubated for a further 6 h. With LDL supplementation providing a fresh source of cholesterol available for esterification, a comparison of free and total cholesterol levels at the end of 6 hours yielded insight into to how much esterified cholesterol was produced by the cells. Unlike healthy cells, which esterify a considerable proportion of the introduced cholesterol pool, NPC1 cells are incapable of transporting cholesterol across the lysosomal membrane to be esterified, and thus retain cholesterol only in its free, unesterified form. However, treatment with NPC1 M1ψTP mRNA 20-24 h prior to LDL supplementation restored the cells' capacity for cholesterol esterification, as evidenced by a significant reduction in free cholesterol levels without a change in total cholesterol levels (FIG. 4D). Confocal imaging analysis corroborated these findings. After carrying out the 48 hours incubation in lipoprotein-deficient serum followed by 6 hours incubation with LDL, the inventors stained cells with either filipin, a dye that binds specifically to free cholesterol (Norman et al (1972)), or Lysotracker Red, a dye that is specific for acidic organelles.

While Lipofectamine-treated GM18393 cells showed a stark increase in filipin and Lysotracker Red staining relative to healthy control cells, mRNA treatment caused a clear reduction in staining intensities (FIG. 4E). Image quantification confirmed that mRNA treatment caused a statistically significant reduction in filipin intensity, with free cholesterol levels essentially reverting to healthy baseline levels (FIG. 4F). The inventors also observed a clear reduction in lysosome size, as measured by Lysotracker Red staining (FIG. 4G). Since NPC1 disease is marked by lysosomal enlargement due to cholesterol entrapment, these data suggest that mRNA treatment causes functional NPC1 protein to be correctly installed in the endo-lysosomal membrane, resulting in the mobilization of trapped cholesterol and normalization of lysosome size. Taken together, these findings show that mRNA treatment has the capacity to rescue NPC1 protein insufficiency and correct the molecular and phenotypic consequences of biallelic NPC1 mutations. The results therefore demonstrate for the first time, that the delivery of exogenous mRNA can be used to rescue the protein insufficiency and pathogenic phenotype caused by NPC1 disease.

### REFERENCES

Altschul et al. (1997) Nucleic Acids Res. 25:3389-3402.
Angov et al. (2008) PLOS ONE 3: e2189.
Andries et al. (2015) Journal of Controlled Release 217:337-344.
Ausubel et al. (1988) Current Protocols in Molecular Biology, Greene Pub. Associates and Wiley-Interscience.
Baiersdörfer et al. (2019) Nucleic Acids 15:26-35.
Brown (1991) Essential Molecular Biology: A Practical Approach, IRL Press, v: 1 & 2.
Carillo et al., SIAM J. Applied Math. 48, 1073 (1988).
Chandler et al. (2017) Human Molecular Genetics 26:52-64.
Coligan et al. Current Protocols in Immunology, John Wiley & Sons (including all updates until present).
Deng (1997) FEBS Letters 409:269-272.
Floor and Doudna (2016) eLife 5: e10921.
Gait (1984) Oligonucleotide synthesis: a practical approach, Oxford (Oxfordshire), Washington, DC: IRL Press.
Glover and Hames (1995 and 1996) DNA Cloning: A Practical Approach, v: 1-4 (1995 and 1996)
Gribskov and Devereux (1991) Sequence Analysis Primer. M Stockton Press, New York.
Griffin and Griffin (1994) Computer Analysis of Sequence Data: Part I, Humana Press, New Jersey.
Hammond et al. (2019) Biochimica et Biophysica Acta (BBA)-Molecular and Cell Biology of Lipids 1864, 1109-1123.
Hastings et al. (2019). Orphanet Journal of Rare Diseases 14:228.
Herdewijn (2005) Methods in Molecular Biology, v.288.
Hia et al. (2019) EMBO reports 20: e48220.
Höglinger et al. (2019) Nature Communications 10:4276.
Hughes et al. (2018) Human Molecular Genetics 27:3079-3098.
Jayarama et al. (2012) Angew. Chem. Int. Ed., 51:8529-8533.
Kirkegaard et al. (2016) Science Translational Medicine 8: 355ra118.
Kwon et al. (2009) Cell 137:1213-1224.
Lesk (1988) Computational Molecular Biology, Oxford University Press, New York.
Li et al. (2016) Bioconjugate Chemistry 27:849-853.
Li et al. (2021) Molecular Therapy 29:691-701,
Liu et al. (2009) Proceedings of the National Academy of Sciences 106:2377.
Lorenz et al. (2011) Algorithms for Molecular Biology 6:26.
Maier et al. (2013) Molecular Therapy 21, 1570-1578.
Mathews et al. (2004) PNAS 101:7287.
Mauger et al. (2019) PNAS 116:24075.
Mauro and Chappell (2014) Trends in Molecular Medicine 20:604-613.
Needelman and Wunsch, (1970) J. Mol. Biol., 48, 443-453.
Nielsen et al. (2011) PLOS ONE 6: e27287.
Norman et al. (1972) Journal of Biological Chemistry 247:1918-1929.
Park et al. (2003) Human Mutation 22:313-325.
Pathan et al. (2009) Recent Patents on Drug Delivery & Formulation 3:71-89.
Patterson et al. (2012). Molecular Genetics and Metabolism 106:330-344.
Patterson et al. (2020) Orphanet Journal of Rare Diseases 15:1-10.
Pentchev et al. (1986) Journal of Biological Chemistry 261:2772-2777.
Perbal (1984) A Practical Guide to Molecular Cloning, John Wiley and Sons,
Qian et al. (2012) PLOS Genetics 8: e1002603.
Raab et al. (2010) Systems and Synthetic Biology 4:215-225.
Remington, J. R., Gennaro, A. R., (1990) Remington's Pharmaceutical Sciences (18th ed., Mack Publishing Co., Easton, Pa)
Sambrook et al. (1989) Molecular Cloning: A Laboratory Manual, Cold Spring Harbour Laboratory Press.
Semple et al. (2010) Nature Biotechnol. 28:172-176
Silvers et al. (2015). ChemBioChem 16, 1109-1114.
Sleat et al. (2004) PNAS 101:5886.
Smith (1993) Biocomputing: Infomatics and Genome Projects. Academic Press, New York.
Solomon et al. (2020) JAMA Neurol. 77, 1564-1568.
Subramanian and Balch (2008) PNAS 105:15223.
Svitkin et al. (2017) Nucleic Acids Research 45, 6023-6036.
Thess et al. (2015) Molecular Therapy 23:1456-1464.
U.S. Pat. No. 7,893,302.
U.S. Pat. No. 7,404,969.
U.S. Pat. No. 8,283,333.
U.S. Pat. No. 8,466,122.
US20100036115.
US20120202871.
US20130064894.
US20130129785.
US20130150625.
US20130178541.
US20130225836.
US20050222064.
U.S. Pharmacopeia (1984): National Formulary (Mack Publishing Company, Easton, Pa.).
Vaidyanathan et al. (2018) Molecular Therapy-Nucleic Acids 12:530-542.
Vanier (2010) Orphanet Journal of Rare Diseases 5:1750-1172.
Verdera et al. (2020) Molecular Therapy 28:723-746.
Villalobos et al. (2006) BMC Bioinformatics 7:285.
von Heinje, G. (1987) Sequence Analysis in Molecular Biology, Academic Press.
Wang et al. (1995) Molecular and Cellular Biology 15:1769.
Wassif et al. (2016). Genetics in Medicine 18:41-48.
WO 2012040184.
WO 2011153120.
WO 2011149733.
WO 2011090965.
WO 2011043913.
WO 2011022460.
WO 2012061259.
WO 2012054365.
WO 2012044638.
WO 2010080724.
WO 201021865.
WO 2008103276.
WO 2013086373.
WO 2013086354.
WO 2015164674.
WO 2013090648.
WO 2011127255.
WO 2008103276.
Wu et al. (2006). Protein Expression and Purification 47:441-445.
Xie et al. (2017) Journal of Lipid Research 58:512-518.
Yanjaninet al. (2010) Neuropsychiatric Genetics 153B: 132-140.

SEQUENCE LIST

Sequence Listing Information:
DTD Version: V1_3
File Name: 536227PCT.xml
Software Name: WIPO Sequence
Software Version: 2.2.0
Production Date: 2023-01-22
General Information:
Current application/Applicant file reference: 536227PCT
Earliest priority application/IP Office: AU
Earliest priority application/Application number: 2022900150
Earliest priority application/Filing date: 2022-01-28
Applicant name: The University of Melbourne
Applicant name/Language: en
Invention title: THERAPEUTIC mRNA (en)
Sequence Total Quantity: 13
Sequences:
Sequence Number (ID): 1
Length: 122
Molecule Type: RNA
Features Location/Qualifiers:
-source, 1..122
>mol_type, mRNA
>organism, Synthetic construct
Residues:

| | |
|---|---|
| gcgcagttcc gccggaagtg gccccagcct cgaggccggg cgtcttcggt catctccggc | 60 |
| gcttctaggg ctggttcccg tcatcttcgg gagccgtgga gctctcggat acagccgaca | 120 |
| cc | 122 |

Sequence Number (ID): 2
Length: 3837
Molecule Type: RNA
Features Location/Qualifiers:
-source, 1..3837
>mol_type, mRNA
>organism, Homo sapiens
Residues:

| | |
|---|---|
| atgaccgccc ggggcctggc cctgggcctg ctgctgctgc tgctgtgccc cgcccaggtg | 60 |
| ttcagccaga gctgcgtgtg gtacggcgag tgcggcatcg cctacggcga caagcggtac | 120 |
| aactgcgagt acagcggccc ccccaagccc ctgcccaagg acggctacga cctggtgcag | 180 |
| gagctgtgcc ccggcttctt cttcggcaac gtgagcctgt gctgcgacgt gcggcagctg | 240 |
| cagaccctga aggacaacct gcagctgccc ctgcagttcc tgagccggtg ccccagctgc | 300 |
| ttctacaacc tgctgaacct gttctgcgag ctgacctgca gcccccggca gagccagttc | 360 |
| ctgaacgtga ccgccaccga ggactacgtg gaccccgtga ccaaccagac caagaccaac | 420 |
| gtgaaggagc tgcagtacta cgtgggccag agcttcgcca acgccatgta caacgcctgc | 480 |
| cgggacgtgg aggcccccag cagcaacgac aaggccctgg gcctgctgtg cggcaaggac | 540 |
| gccgacgcct gcaacgccac caactggatc gagtacatgt tcaacaagga caacggccag | 600 |
| gcccccttca ccatcacccc cgtgttcagc gacttccccg tgcacggcat ggagcccatg | 660 |
| aacaacgcca ccaagggctg cgacgagagc gtggacgagg tgaccgcccc ctgcagctgc | 720 |
| caggactgca gcatcgtgtg cggccccaag ccccagccgc cgccgccgcc ggccccctgg | 780 |
| accatcctgg gcctggacgc catgtacgtg atcatgtgga tcacctacat ggccttcctg | 840 |
| ctggtgttct tcggcgcctt cttcgccgtg tggtgctacc ggaagcggta cttcgtgagc | 900 |
| gagtacaccc ccatcgacag caacatcgcc ttcagcgtga acgccagcga caagggcgag | 960 |
| gccagctgct gcgaccccgt gagcgccgcc ttcgagggct gcctgcggcg gctgttcacc | 1020 |
| cggtggggca gcttctgcgt gcggaacccc ggctgcgtga tcttcttcag cctggtgttc | 1080 |
| atcaccgcct gcagcagcgg cctggtgttc gtgcgggtga ccaccaaccc cgtggacctg | 1140 |
| tggagcgccc ccagcagcca ggcccggctg gagaaggagt acttcgacca gcacttcggc | 1200 |
| cccttcttcc ggaccgagca gctgatcatc cgggcccccc tgaccgacaa gcacatctac | 1260 |
| cagccctacc ccagcggcgc cgacgtgccc ttcggccccc ccctggacat ccagatcctg | 1320 |
| caccaggtgc tggacctgca gatcgccatc gagaacatca ccgccagcta cgacaacgag | 1380 |
| accgtgaccc tgcaggacat ctgcctggcc cccctgagcc cctacaacac caactgcacc | 1440 |
| atcctgagcg tgctgaacta cttccagaac agccacagcg tgctggacca caagaagggc | 1500 |
| gacgacttct tcgtgtacgc cgactaccac acccacttcc tgtactgcgt gcgggccccc | 1560 |
| gccagcctga acgacaccag cctgctgcac gacccctgcc tgggcacctt cggcggcccc | 1620 |
| gtgttcccct ggctggtgct gggcggctac gacgaccaga actacaacaa cgccaccgcc | 1680 |
| ctggtgatca ccttccccgt gaacaactac tacaacgaca ccgagaagct gcagcgggcc | 1740 |
| caggcctggg agaaggagtt catcaacttc gtgaagaact acaagaaccc caacctgacc | 1800 |
| atcagcttca ccgccgagcg gagcatcgag gacgagctga accgggagag cgacagcgac | 1860 |
| gtgttcaccg tggtgatcag ctacgccatc atgttcctgt acatcagcct ggccctgggc | 1920 |
| cacatgaaga gctgccggcg gctgctggtg gacagcaagg tgagcctggg catcgccggc | 1980 |
| atcctgatcg tgctgagcag cgtggcctgc agcctgggcg tgttcagcta catcggcctg | 2040 |
| cccctgaccc tgatcgtgat cgaggtgatc cccttcctgg tgctggccgt gggcgtggac | 2100 |
| aacatcttca tcctggtgca ggcctaccag cgggacgagc ggctgcaggg cgagaccctg | 2160 |
| gaccagcagc tgggccgggt gctgggcgag gtggccccca gcatgttcct gagcagcttc | 2220 |
| agcgagaccg tggccttctt cctgggcgcc ctgagcgtga tgcccgccgt gcacaccttc | 2280 |
| agcctgttcg ccggcctggc cgtgttcatc gacttcctgc tgcagatcac ctgcttcgtg | 2340 |
| agcctgctgg gcctggacat caagcggcag gagaagaacc ggctggacat cttctgctgc | 2400 |
| gtgcggggcg ccgaggacgg caccagcgtg caggccagcg agagctgcct gttccggttc | 2460 |
| ttcaagaaca gctacagccc cctgctgctg aaggactgga tgcggcccat cgtgatcgcc | 2520 |
| atcttcgtgg gcgtgctgag cttcagcatc gccgtgctga acaaggtgga catcggcctg | 2580 |
| gaccagagcc tgagcatgcc cgacgacagc tacatggtgg actacttcaa gagcatcagc | 2640 |
| cagtacctgc acgccggccc ccccgtgtac ttcgtgctgg aggagggcca cgactacacc | 2700 |
| agcagcaagg gccagaacat ggtgtgcggc ggcatgggct gcaacaacga cagcctggtg | 2760 |
| cagcagatct tcaacgccgc ccagctggac aactacaccc ggatcggctt cgcccccagc | 2820 |
| agctggatcg acgactactt cgactgggtg aagccccaga gcagctgctg ccgggtggac | 2880 |
| aacatcaccg accagttctg caacgccagc gtggtggacc ccgcctgcgt gcggtgccgg | 2940 |
| cccctgaccc ccgagggcaa gcagcggccc cagggcggcg acttcatgcg gttcctgccc | 3000 |
| atgttcctga gcgacaaccc caaccccaag tgcggcaagg gcggccacgc cgcctacagc | 3060 |
| agcgccgtga acatcctgct gggccacggc acccgggtgg gcgccaccta cttcatgacc | 3120 |
| taccacaccg tgctgcagac cagcgccgac ttcatcgacg ccctgaagaa ggcccggctg | 3180 |
| atcgccagca acgtgaccga gaccatgggc atcaacggca gcgcctaccg ggtgttcccc | 3240 |
| tacagcgtgt tctacgtgtt ctacgagcag tacctgacca tcatcgacga caccatcttc | 3300 |
| aacctgggcg tgagcctggg cgccatcttc ctggtgacca tggtgctgct gggctgcgag | 3360 |
| ctgtggagcg ccgtgatcat gtgcgccacc atcgccatgg tgctggtgaa catgttcggc | 3420 |
| gtgatgtggc tgtggggcat cagcctgaac gccgtgagcc tggtgaacct ggtgatgagc | 3480 |
| tgcggcatca gcgtggagtt ctgcagccac atcacccggg ccttcaccgt gagcatgaag | 3540 |
| ggcagccggg tggagcgggc cgaggaggcc ctggcccaca tgggcagcag cgtgttcagc | 3600 |
| ggcatcaccc tgaccaagtt cggcggcatc gtggtgctgg ccttcgccaa gagccagatc | 3660 |
| ttccagatct tctacttccg gatgtacctg gccatggtgc tgctgggcgc cacccacggc | 3720 |
| ctgatcttcc tgcccgtgct gctgagctac atcggcccca gcgtgaacaa ggccaagagc | 3780 |
| tgcgccaccg aggagcggta caagggcacc gagcgggagc ggctgctgaa cttctga | 3837 |

Sequence Number (ID): 3
Length: 111
Molecule Type: RNA
Features Location/Qualifiers:
-source, 1..111
>mol_type, mRNA
>organism, Synthetic construct
Residues:

| | |
|---|---|
| gctggagcct cggtggccat gcttcttgcc ccttgggcct ccccccagcc cctcctcccc | 60 |
| ttcctgcacc cgtacccccg tggtctttga ataaagtctg agtgggcggc a | 111 |

Sequence Number (ID): 4
Length: 100
Molecule Type: RNA
Features Location/Qualifiers:
-source, 1..100
>mol_type, mRNA
>organism, Synthetic construct
Residues:

| | |
|---|---|
| aaaaaaaaaa aaaaaaaaaa aaaaaaaaaa aaaaaaaaaa aaaaaaaaaa aaaaaaaaaa | 60 |
| aaaaaaaaaa aaaaaaaaaa aaaaaaaaaa aaaaaaaaaa | 100 |

Sequence Number (ID): 5
Length: 4172
Molecule Type: RNA
Features Location/Qualifiers:
-source, 1..4172
>mol_type, mRNA
>organism, Synthetic construct
Residues:

| | |
|---|---|
| aggcgcagtt ccgccggaag tggccccagc ctcgaggccg ggcgtcttcg gtcatctccg | 60 |
| gcgcttctag ggctggttcc cgtcatcttc gggagccgtg gagctctcgg atacagccga | 120 |
| caccatgacc gcccggggcc tggccctggg cctgctgctg ctgctgctgt gccccgccca | 180 |
| ggtgttcagc cagagctgcg tgtggtacgg cgagtgcggc atcgcctacg gcgacaagcg | 240 |
| gtacaactgc gagtacagcg gcccccccaa gcccctgccc aaggacggct acgacctggt | 300 |
| gcaggagctg tgccccggct tcttcttcgg caacgtgagc ctgtgctgcg acgtgcggca | 360 |
| gctgcagacc ctgaaggaca acctgcagct gcccctgcag ttcctgagcc ggtgccccag | 420 |
| ctgcttctac aacctgctga acctgttctg cgagctgacc tgcagccccc ggcagagcca | 480 |
| gttcctgaac gtgaccgcca ccgaggacta cgtggacccc gtgaccaacc agaccaagac | 540 |
| caacgtgaag gagctgcagt actacgtggg ccagagcttc gccaacgcca tgtacaacgc | 600 |
| ctgccgggac gtggaggccc ccagcagcaa cgacaaggcc ctgggcctgc tgtgcggcaa | 660 |
| ggacgccgac gcctgcaacg ccaccaactg gatcgagtac atgttcaaca aggacaacgg | 720 |
| ccaggccccc ttcaccatca cccccgtgtt cagcgacttc cccgtgcacg gcatggagcc | 780 |
| catgaacaac gccaccaagg gctgcgacga gagcgtggac gaggtgaccg ccccctgcag | 840 |
| ctgccaggac tgcagcatcg tgtgcggccc caagccccag ccgccgccgc cgccggcccc | 900 |
| ctggaccatc ctgggcctgg acgccatgta cgtgatcatg tggatcacct acatggcctt | 960 |
| cctgctggtg ttcttcggcg ccttcttcgc cgtgtggtgc taccggaagc ggtacttcgt | 1020 |
| gagcgagtac acccccatcg acagcaacat cgccttcagc gtgaacgcca gcgacaaggg | 1080 |
| cgaggccagc tgctgcgacc ccgtgagcgc cgccttcgag ggctgcctgc ggcggctgtt | 1140 |
| cacccggtgg ggcagcttct gcgtgcggaa ccccggctgc gtgatcttct tcagcctggt | 1200 |
| gttcatcacc gcctgcagca gcggcctggt gttcgtgcgg gtgaccacca accccgtgga | 1260 |
| cctgtggagc gcccccagca gccaggcccg gctggagaag gagtacttcg accagcactt | 1320 |
| cggccccttc ttccggaccg agcagctgat catccgggcc cccctgaccg acaagcacat | 1380 |
| ctaccagccc taccccagcg gcgccgacgt gcccttcggc ccccccctgg acatccagat | 1440 |
| cctgcaccag gtgctggacc tgcagatcgc catcgagaac atcaccgcca gctacgacaa | 1500 |
| cgagaccgtg accctgcagg acatctgcct ggcccccctg agcccctaca acaccaactg | 1560 |
| caccatcctg agcgtgctga actacttcca gaacagccac agcgtgctgg accacaagaa | 1620 |
| gggcgacgac ttcttcgtgt acgccgacta ccacacccac ttcctgtact gcgtgcgggc | 1680 |
| ccccgccagc ctgaacgaca ccagcctgct gcacgacccc tgcctgggca ccttcggcgg | 1740 |
| ccccgtgttc ccctggctgg tgctgggcgg ctacgacgac cagaactaca acaacgccac | 1800 |
| cgccctggtg atcaccttcc ccgtgaacaa ctactacaac gacaccgaga agctgcagcg | 1860 |
| ggcccaggcc tgggagaagg agttcatcaa cttcgtgaag aactacaaga accccaacct | 1920 |
| gaccatcagc ttcaccgccg agcggagcat cgaggacgag ctgaaccggg agagcgacag | 1980 |
| cgacgtgttc accgtggtga tcagctacgc catcatgttc ctgtacatca gcctggccct | 2040 |
| gggccacatg aagagctgcc ggcggctgct ggtggacagc aaggtgagcc tgggcatcgc | 2100 |
| cggcatcctg atcgtgctga gcagcgtggc ctgcagcctg ggcgtgttca gctacatcgg | 2160 |
| cctgcccctg accctgatcg tgatcgaggt gatccccttc ctggtgctgg ccgtgggcgt | 2220 |
| ggacaacatc ttcatcctgg tgcaggccta ccagcgggac gagcggctgc agggcgagac | 2280 |
| cctggaccag cagctgggcc gggtgctggg cgaggtggcc cccagcatgt tcctgagcag | 2340 |
| cttcagcgag accgtggcct tcttcctggg cgccctgagc gtgatgcccg ccgtgcacac | 2400 |
| cttcagcctg ttcgccggcc tggccgtgtt catcgacttc ctgctgcaga tcacctgctt | 2460 |
| cgtgagcctg ctgggcctgg acatcaagcg gcaggagaag aaccggctgg acatcttctg | 2520 |
| ctgcgtgcgg ggcgccgagg acggcaccag cgtgcaggcc agcgagagct gcctgttccg | 2580 |
| gttcttcaag aacagctaca gccccctgct gctgaaggac tggatgcggc ccatcgtgat | 2640 |
| cgccatcttc gtgggcgtgc tgagcttcag catcgccgtg ctgaacaagg tggacatcgg | 2700 |
| cctggaccag agcctgagca tgcccgacga cagctacatg gtggactact tcaagagcat | 2760 |
| cagccagtac ctgcacgccg gcccccccgt gtacttcgtg ctggaggagg gccacgacta | 2820 |
| caccagcagc aagggccaga acatggtgtg cggcggcatg ggctgcaaca acgacagcct | 2880 |
| ggtgcagcag atcttcaacg ccgcccagct ggacaactac acccggatcg gcttcgcccc | 2940 |
| cagcagctgg atcgacgact acttcgactg ggtgaagccc cagagcagct gctgccgggt | 3000 |
| ggacaacatc accgaccagt tctgcaacgc cagcgtggtg gaccccgcct gcgtgcggtg | 3060 |
| ccggcccctg acccccgagg gcaagcagcg gccccagggc ggcgacttca tgcggttcct | 3120 |
| gcccatgttc ctgagcgaca accccaaccc caagtgcggc aagggcggcc acgccgccta | 3180 |
| cagcagcgcc gtgaacatcc tgctgggcca cggcacccgg gtgggcgcca cctacttcat | 3240 |
| gacctaccac accgtgctgc agaccagcgc cgacttcatc gacgccctga agaaggcccg | 3300 |
| gctgatcgcc agcaacgtga ccgagaccat gggcatcaac ggcagcgcct accgggtgtt | 3360 |
| cccctacagc gtgttctacg tgttctacga gcagtacctg accatcatcg acgacaccat | 3420 |
| cttcaacctg ggcgtgagcc tgggcgccat cttcctggtg accatggtgc tgctgggctg | 3480 |
| cgagctgtgg agcgccgtga tcatgtgcgc caccatcgcc atggtgctgg tgaacatgtt | 3540 |
| cggcgtgatg tggctgtggg gcatcagcct gaacgccgtg agcctggtga acctggtgat | 3600 |
| gagctgcggc atcagcgtgg agttctgcag ccacatcacc cgggccttca ccgtgagcat | 3660 |
| gaagggcagc cgggtggagc gggccgagga ggccctggcc cacatgggca gcagcgtgtt | 3720 |
| cagcggcatc accctgacca agttcggcgg catcgtggtg ctggccttcg ccaagagcca | 3780 |
| gatcttccag atcttctact tccggatgta cctggccatg gtgctgctgg gcgccaccca | 3840 |
| cggcctgatc ttcctgcccg tgctgctgag ctacatcggc cccagcgtga acaaggccaa | 3900 |
| gagctgcgcc accgaggagc ggtacaaggg caccgagcgg gagcggctgc tgaacttctg | 3960 |
| agctggagcc tcggtggcca tgcttcttgc cccttgggcc tccccccagc ccctcctccc | 4020 |
| cttcctgcac ccgtaccccc gtggtctttg aataaagtct gagtgggcgg caaaaaaaaa | 4080 |
| aaaaaaaaaa aaaaaaaaaa aaaaaaaaaa aaaaaaaaaa aaaaaaaaaa aaaaaaaaaa | 4140 |
| aaaaaaaaaa aaaaaaaaaa aaaaaaaaaa aa | 4172 |

Sequence Number (ID): 6
Length: 1653
Molecule Type: RNA
Features Location/Qualifiers:
-source, 1..1653
>mol_type, mRNA
>organism, Photinus pyralis
Residues:

| | |
|---|---|
| atggaggacg ccaagaacat caagaagggc cccgccccct tctaccccct ggaggacggc | 60 |
| accgccggcg agcagctgca caaggccatg aagcggtacg ccctggtgcc cggcaccatc | 120 |
| gccttcaccg acgcccacat cgaggtggac atcacctacg ccgagtactt cgagatgagc | 180 |
| gtgcggctgg ccgaggccat gaagcggtac ggcctgaaca ccaaccaccg gatcgtggtg | 240 |
| tgcagcgaga acagcctgca gttcttcatg cccgtgctgg gcgccctgtt catcggcgtg | 300 |
| gccgtggccc ccgccaacga catctacaac gagcgggagc tgctgaacag catgggcatc | 360 |
| agccagccca ccgtggtgtt cgtgagcaag aagggcctgc agaagatcct gaacgtgcag | 420 |
| aagaagctgc ccatcatcca gaagatcatc atcatggaca gcaagaccga ctaccagggc | 480 |
| ttccagagca tgtacacctt cgtgaccagc cacctgcccc ccggcttcaa cgagtacgac | 540 |
| ttcgtgcccg agagcttcga ccgggacaag accatcgccc tgatcatgaa cagcagcggc | 600 |
| agcaccggcc tgcccaaggg cgtggccctg ccccaccgga ccgcctgcgt gcggttcagc | 660 |
| cacgcccggg accccatctt cggcaaccag atcatccccg acaccgccat cctgagcgtg | 720 |
| gtgcccttcc accacggctt cggcatgttc accaccctgg gctacctgat ctgcggcttc | 780 |
| cgggtggtgc tgatgtaccg gttcgaggag gagctgttcc tgcggagcct gcaggactac | 840 |
| aagatccaga gcgccctgct ggtgcccacc ctgttcagct tcttcgccaa gagcaccctg | 900 |
| atcgacaagt acgacctgag caacctgcac gagatcgcca gcggcggcgc ccccctgagc | 960 |
| aaggaggtgg gcgaggccgt ggccaagcgg ttccacctgc ccggcatccg gcagggctac | 1020 |
| ggcctgaccg agaccaccag cgccatcctg atcacccccg agggcgacga caagcccggc | 1080 |
| gccgtgggca aggtggtgcc cttcttcgag gccaaggtgg tggacctgga caccggcaag | 1140 |
| accctgggcg tgaaccagcg gggcgagctg tgcgtgcggg gccccatgat catgagcggc | 1200 |
| tacgtgaaca accccgaggc caccaacgcc ctgatcgaca aggacggctg gctgcacagc | 1260 |
| ggcgacatcg cctactggga cgaggacgag cacttcttca tcgtggaccg gctgaagagc | 1320 |
| ctgatcaagt acaagggcta ccaggtggcc cccgccgagc tggagagcat cctgctgcag | 1380 |
| caccccaaca tcttcgacgc cggcgtggcc ggcctgcccg acgacgacgc cggcgagctg | 1440 |
| cccgccgccg tggtggtgct ggagcacggc aagaccatga ccgagaagga gatcgtggac | 1500 |
| tacgtggcca gccaggtgac caccgccaag aagctgcggg gcggcgtggt gttcgtggac | 1560 |
| gaggtgccca agggcctgac cggcaagctg gacgcccgga agatccggga gatcctgatc | 1620 |
| aaggccaaga agggcggcaa gatcgccgtg tga | 1653 |

Sequence Number (ID): 7
Length: 1988
Molecule Type: RNA
Features Location/Qualifiers:
-source, 1..1988
>mol_type, mRNA
>organism, Synthetic construct
Residues:

| | |
|---|---|
| aggcgcagtt ccgccggaag tggccccagc ctcgaggccg ggcgtcttcg gtcatctccg | 60 |
| gcgcttctag ggctggttcc cgtcatcttc gggagccgtg gagctctcgg atacagccga | 120 |
| caccatggag gacgccaaga acatcaagaa gggccccgcc cccttctacc ccctggagga | 180 |
| cggcaccgcc ggcgagcagc tgcacaaggc catgaagcgg tacgccctgg tgcccggcac | 240 |
| catcgccttc accgacgccc acatcgaggt ggacatcacc tacgccgagt acttcgagat | 300 |
| gagcgtgcgg ctggccgagg ccatgaagcg gtacggcctg aacaccaacc accggatcgt | 360 |
| ggtgtgcagc gagaacagcc tgcagttctt catgcccgtg ctgggcgccc tgttcatcgg | 420 |
| cgtggccgtg gcccccgcca acgacatcta caacgagcgg gagctgctga acagcatggg | 480 |
| catcagccag cccaccgtgg tgttcgtgag caagaagggc ctgcagaaga tcctgaacgt | 540 |
| gcagaagaag ctgcccatca tccagaagat catcatcatg gacagcaaga ccgactacca | 600 |
| gggcttccag agcatgtaca ccttcgtgac cagccacctg ccccccggct tcaacgagta | 660 |
| cgacttcgtg cccgagagct tcgaccggga caagaccatc gccctgatca tgaacagcag | 720 |
| cggcagcacc ggcctgccca agggcgtggc cctgccccac cggaccgcct gcgtgcggtt | 780 |
| cagccacgcc cgggacccca tcttcggcaa ccagatcatc cccgacaccg ccatcctgag | 840 |
| cgtggtgccc ttccaccacg gcttcggcat gttcaccacc ctgggctacc tgatctgcgg | 900 |
| cttccgggtg gtgctgatgt accggttcga ggaggagctg ttcctgcgga gcctgcagga | 960 |
| ctacaagatc cagagcgccc tgctggtgcc caccctgttc agcttcttcg ccaagagcac | 1020 |
| cctgatcgac aagtacgacc tgagcaacct gcacgagatc gccagcggcg gcgcccccct | 1080 |
| gagcaaggag gtgggcgagg ccgtggccaa gcggttccac ctgcccggca tccggcaggg | 1140 |
| ctacggcctg accgagacca ccagcgccat cctgatcacc cccgagggcg acgacaagcc | 1200 |
| cggcgccgtg ggcaaggtgg tgcccttctt cgaggccaag gtggtggacc tggacaccgg | 1260 |
| caagaccctg ggcgtgaacc agcggggcga gctgtgcgtg cggggcccca tgatcatgag | 1320 |
| cggctacgtg aacaaccccg aggccaccaa cgccctgatc gacaaggacg gctggctgca | 1380 |
| cagcggcgac atcgcctact gggacgagga cgagcacttc ttcatcgtgg accggctgaa | 1440 |
| gagcctgatc aagtacaagg gctaccaggt ggcccccgcc gagctggaga gcatcctgct | 1500 |
| gcagcacccc aacatcttcg acgccggcgt ggccggcctg cccgacgacg acgccggcga | 1560 |
| gctgcccgcc gccgtggtgg tgctggagca cggcaagacc atgaccgaga aggagatcgt | 1620 |
| ggactacgtg gccagccagg tgaccaccgc caagaagctg cggggcggcg tggtgttcgt | 1680 |
| ggacgaggtg cccaagggcc tgaccggcaa gctggacgcc cggaagatcc gggagatcct | 1740 |
| gatcaaggcc aagaagggcg gcaagatcgc cgtgtgagct ggagcctcgg tggccatgct | 1800 |
| tcttgcccct tgggcctccc cccagcccct cctccccttc ctgcacccgt acccccgtgg | 1860 |
| tctttgaata aagtctgagt gggcggcaaa aaaaaaaaaa aaaaaaaaaa aaaaaaaaaa | 1920 |
| aaaaaaaaaa aaaaaaaaaa aaaaaaaaaa aaaaaaaaaa aaaaaaaaaa aaaaaaaaaa | 1980 |
| aaaaaaaa | 1988 |

Sequence Number (ID): 8
Length: 3837
Molecule Type: RNA
Features Location/Qualifiers:
-source, 1..3837
>mol_type, mRNA
>organism, Homo sapiens
Residues:

| | |
|---|---|
| atgaccgctc gcggcctggc ccttggcctc ctcctgctgc tactgtgtcc agcgcaggtg | 60 |
| ttttcacagt cctgtgtttg gtatggagag tgtggaattg catatgggga caagaggtac | 120 |
| aattgcgaat attctggccc accaaaacca ttgccaaagg atggatatga cttagtgcag | 180 |
| gaactctgtc caggattctt ctttggcaat gtcagtctct gttgtgatgt tcggcagctt | 240 |
| cagacactaa aagacaacct gcagctgcct ctacagtttc tgtccagatg tccatcctgt | 300 |
| ttttataacc tactgaacct gttttgtgag ctgacatgta gccctcgaca gagtcagttt | 360 |
| ttgaatgtta cagctactga agattatgtt gatcctgtta caaaccagac gaaaacaaat | 420 |
| gtgaaagagt tacaatacta cgtcggacag agttttgcca atgcaatgta caatgcctgc | 480 |
| cgggatgtgg aggccccctc aagtaatgac aaggccctgg gactcctgtg tgggaaggac | 540 |
| gctgacgcct gtaatgccac caactggatt gaatacatgt tcaataagga caatggacag | 600 |
| gcacctttta ccatcactcc tgtgttttca gattttccag tccatgggat ggagcccatg | 660 |
| aacaatgcca ccaaaggctg tgacgagtct gtggatgagg tcacagcacc atgtagctgc | 720 |
| caagactgct ctattgtctg tggccccaag ccccagcccc cacctcctcc tgctccctgg | 780 |
| acgatccttg gcttggacgc catgtatgtc atcatgtgga tcacctacat ggcgtttttg | 840 |
| cttgtgtttt ttggagcatt ttttgcagtg tggtgctaca gaaaacggta ttttgtctcc | 900 |
| gagtacactc ccatcgatag caatatagct ttttctgtta atgcaagtga caaaggagag | 960 |
| gcgtcctgct gtgaccctgt cagcgcagca tttgagggct gcttgaggcg gctgttcaca | 1020 |
| cgctgggggt ctttctgcgt ccgaaaccct ggctgtgtca ttttcttctc gctggtcttc | 1080 |
| attactgcgt gttcgtcagg cctggtgttt gtccgggtca caaccaatcc agttgacctc | 1140 |
| tggtcagccc ccagcagcca ggctcgcctg gaaaaagagt actttgacca gcactttggg | 1200 |
| cctttcttcc ggacggagca gctcatcatc cgggcccctc tcactgacaa acacatttac | 1260 |
| cagccatacc cttcgggagc tgatgtaccc tttggacctc cgcttgacat acagatactg | 1320 |
| caccaggttc ttgacttaca aatagccatc gaaaacatta ctgcctctta tgacaatgag | 1380 |
| actgtgacac ttcaagacat ctgcttggcc cctctttcac cgtataacac gaactgcacc | 1440 |
| attttgagtg tgttaaatta cttccagaac agccattccg tgctggacca caagaaaggg | 1500 |
| gacgacttct ttgtgtatgc cgattaccac acgcactttc tgtactgcgt acgggctcct | 1560 |
| gcctctctga atgatacaag tttgctccat gacccttgtc tgggtacgtt tggtggacca | 1620 |
| gtgttcccgt ggcttgtgtt gggaggctat gatgatcaaa actacaataa cgccactgcc | 1680 |
| cttgtgatta ccttccctgt caataattac tataatgata cagagaagct ccagagggcc | 1740 |
| caggcctggg aaaaagagtt tattaatttt gtgaaaaact acaagaatcc caatctgacc | 1800 |
| atttccttca ctgctgaacg aagtattgaa gatgaactaa atcgtgaaag tgacagtgat | 1860 |
| gtcttcaccg ttgtaattag ctatgccatc atgtttctat atatttccct agccttgggg | 1920 |
| cacatgaaaa gctgtcgcag gcttctggtg gattcgaagg tctcactagg catcgcgggc | 1980 |
| atcttgatcg tgctgagctc ggtggcttgc tccttgggtg tcttcagcta cattgggttg | 2040 |
| cccttgaccc tcattgtgat tgaagtcatc ccgttcctgg tgctggctgt tggagtggac | 2100 |
| aacatcttca ttctggtgca ggcctaccag agagatgaac gtcttcaagg ggaaaccctg | 2160 |
| gatcagcagc tgggcagggt cctaggagaa gtggctccca gtatgttcct gtcatccttt | 2220 |
| tctgagactg tagcattttt cttaggagca ttgtccgtga tgccagccgt gcacaccttc | 2280 |
| tctctctttg cgggattggc agtcttcatt gactttcttc tgcagattac ctgtttcgtg | 2340 |
| agtctcttgg ggttagacat taaacgtcaa gagaaaaatc ggctagacat cttttgctgt | 2400 |
| gtcagaggtg ctgaagatgg aacaagcgtc caggcctcag agagctgttt gtttcgcttc | 2460 |
| ttcaaaaact cctattctcc acttctgcta aaggactgga tgagaccaat tgtgatagca | 2520 |
| atatttgtgg gtgttctgtc attcagcatc gcagtcctga acaaagtaga tattggattg | 2580 |
| gatcagtctc tttcgatgcc agatgactcc tacatggtgg attatttcaa atccatcagt | 2640 |
| cagtacctgc atgcgggtcc gcctgtgtac tttgtcctgg aggaagggca cgactacact | 2700 |
| tcttccaagg ggcagaacat ggtgtgcggc ggcatgggct gcaacaatga ttccctggtg | 2760 |
| cagcagatat ttaacgcggc gcagctggac aactataccc gaataggctt cgccccctcg | 2820 |
| tcctggatcg acgattattt cgactgggtg aagccacagt cgtcttgctg tcgagtggac | 2880 |
| aatatcactg accagttctg caatgcttca gtggttgacc ctgcctgcgt tcgctgcagg | 2940 |
| cctctgactc cggaaggcaa acagaggcct caggggggag acttcatgag attcctgccc | 3000 |
| atgttccttt cggataaccc taaccccaag tgtggcaaag ggggacatgc tgcctatagt | 3060 |
| tctgcagtta acatcctcct tggccatggc accagggtcg gagccacgta cttcatgacc | 3120 |
| taccacaccg tgctgcagac ctctgctgac tttattgacg ctctgaagaa agcccgactt | 3180 |
| atagccagta atgtcaccga aaccatgggc attaacggca gtgcctaccg agtatttcct | 3240 |
| tacagtgtgt tttatgtctt ctacgaacag tacctgacca tcattgacga cactatcttc | 3300 |
| aacctcggtg tgtccctggg cgcgatattt ctggtgacca tggtcctcct gggctgtgag | 3360 |
| ctctggtctg cagtcatcat gtgtgccacc atcgccatgg tcttggtcaa catgtttgga | 3420 |
| gttatgtggc tctggggcat cagtctgaac gctgtatcct tggtcaacct ggtgatgagc | 3480 |
| tgtggcatct ccgtggagtt ctgcagccac ataaccagag cgttcacggt gagcatgaaa | 3540 |
| ggcagccgcg tggagcgcgc ggaagaggca cttgcccaca tgggcagctc cgtgttcagt | 3600 |
| ggaatcacac ttacaaaatt tggagggatt gtggtgttgg cttttgccaa atctcaaatt | 3660 |
| ttccagatat tctacttcag gatgtatttg gccatggtct tactgggagc cactcacgga | 3720 |
| ttaatatttc tccctgtctt actcagttac atagggccat cagtaaataa agccaaaagt | 3780 |
| tgtgccactg aagagcgata caaaggaaca gagcgcgaac ggcttctaaa tttctag | 3837 |

Sequence Number (ID): 9
Length: 525
Molecule Type: RNA
Features Location/Qualifiers:
-source, 1..525
>mol_type,_mRNA
>organism, Homo sapiens
Residues:

| | |
|---|---|
| atgcgtttcc tggcagctac attcctgctc ctggcgctca gcaccgctgc ccaggccgaa | 60 |
| ccggtgcagt tcaaggactg cggttctgtg gatggagtta taaaggaagt gaatgtgagc | 120 |
| ccatgcccca cccaaccctg ccagctgagc aaaggacagt cttacagcgt caatgtcacc | 180 |
| ttcaccagca atattcagtc taaaagcagc aaggccgtgg tgcatggcat cctgatgggc | 240 |
| gtcccagttc cctttcccat tcctgagcct gatggttgta agagtggaat taactgccct | 300 |
| atccaaaaag acaagaccta tagctacctg aataaactac cagtgaaaag cgaatatccc | 360 |
| tctataaaac tggtggtgga gtggcaactt caggatgaca aaaaccaaag tctcttctgc | 420 |
| tgggaaatcc cagtacagat cgtaagtcta tctgggggtg agagggcatg ggtggaggga | 480 |
| agaaagtgga ggagaaatca gactgaaact aaatcagtgc cataa | 525 |

Sequence Number (ID): 10
Length: 1278
Molecule Type: AA
Features Location/Qualifiers:
-source, 1..1278
>mol_type,_mRNA
>organism, Homo sapiens
Residues:

| | |
|---|---|
| MTARGLALGL LLLLLCPAQV FSQSCVWYGE CGIAYGDKRY NCEYSGPPKP LPKDGYDLVQ | 60 |
| ELCPGFFFGN VSLCCDVRQL QTLKDNLQLP LQFLSRCPSC FYNLLNLFCE LTCSPRQSQF | 120 |
| LNVTATEDYV DPVTNQTKTN VKELQYYVGQ SFANAMYNAC RDVEAPSSND KALGLLCGKD | 180 |
| ADACNATNWI EYMFNKDNGQ APFTITPVES DFPVHGMEPM NNATKGCDES VDEVTAPCSC | 240 |
| QDCSIVCGPK PQPPPPPAPW TILGLDAMYV IMWITYMAFL LVFFGAFFAV WCYRKRYFVS | 300 |
| EYTPIDSNIA FSVNASDKGE ASCCDPVSAA FEGCLRRLFT RWGSFCVRNP GCGIFFSLVF | 360 |
| ITACSSGLVF VRVTTNPVDL WSAPSSQARL EKEYFDQHFG PFFRTEQLII RAPLTDKHIY | 420 |
| QPYPSGADVP FGPPLDIQIL HQVLDLQIAI ENITASYDNE TVTLQDICLA PLSPYNTNCT | 480 |
| ILSVLNYFQN SHSVLDHKKG DDFFVYADYH THFLYCVRAP ASLNDTSLLH DPCLGTFGGP | 540 |
| VFPWLVLGGY DDQNYNNATA LVITFPVNNY YNDTEKLQRA QAWEKEFINF VKNYKNPNLT | 600 |
| ISFTAERSIE DELNRESDSD VFTVVISYAI MFLYISLALG HMKSCRRLLV DSKVSLGIAG | 660 |
| ILIVLSSVAC SLGVFSYIGL PLTLIVIEVI PFLVLAVGVD NIFILVQAYQ RDERLQGETL | 720 |
| DQQLGRVLGE VAPSMFLSSF SETVAFFLGA LSVMPAVHTF SLFAGLAVFI DFLLQITCFV | 780 |
| SLLGLDIKRQ EKNRLDIFCC VRGAEDGTSV QASESCLFRF FKNSYSPLLL KDWMRPIVIA | 840 |
| IFVGVLSFSI AVLNKVDIGL DQSLSMPDDS YMVDYFKSIS QYLHAGPPVY FVLEEGHDYT | 900 |
| SSKGQNMVCG GMGCNNDSLV QQIFNAAQLD NYTRIGFAPS SWIDDYFDWV KPQSSCCRVD | 960 |
| NITDQFCNAS VVDPACVRCR PLTPEGKQRP QGGDFMRFLP MFLSDNPNPK CGKGGHAAYS | 1020 |
| SAVNILLGHG TRVGATYFMT YHTVLQTSAD FIDALKKARL IASNVTETMG INGSAYRVFP | 1080 |
| YSVFYVFYEQ YLTIIDDTIF NLGVSLGAIF LVTMVLLGCE LWSAVIMCAT IAMVLVNMFG | 1140 |
| VMWLWGISLN AVSLVNLVMS CGISVEFCSH ITRAFTVSMK GSRVERAEEA LAHMGSSVFS | 1200 |
| GITLTKFGGI VVLAFAKSQI FQIFYFRMYL AMVLLGATHG LIFLPVLLSY IGPSVNKAKS | 1268 |
| CATEERYKGT ERERLLNF | 1278 |

Sequence Number (ID): 11
Length: 174
Molecule Type: AA
Features Location/Qualifiers :
-source, 1..174
>mol_type,_mRNA
>organism, Homo sapiens
Residues:

| | |
|---|---|
| MRFLAATFLL LALSTAAQAE PVQFKDCGSV DGVIKEVNVS PCPTQPCQLS KGQSYSVNVT | 60 |
| FTSNIQSKSS KAVVHGILMG VPVPFPIPEP DGCKSGINCP IQKDKTYSYL NKLPVKSEYP | 120 |
| SIKLVVEWQL QDDKNQSLFC WEIPVQIVSL SGGERAWVEG RKWRRNQTET KSVP | 174 |

Sequence Number (ID): 12
Length: 29
Molecule Type: DNA
Features Location/Qualifiers:
-source, 1..29
>mol_type, other DNA
>organism, Synthetic construct
Residues:

| | |
|---|---|
| ccacgaatct aatacgactc actataagg | 29 |

Sequence Number (ID): 13
Length: 122
Molecule Type: DNA
Features Location/Qualifiers:
-source, 1..122
>mol_type, other DNA
>organism, Synthetic construct
Residues:

| | |
|---|---|
| tttttttttt tttttttttt tttttttttt tttttttttt tttttttttt tttttttttt | 60 |
| tttttttttt tttttttttt tttttttttt tttttttttt gccgcccact cagactttat | 120 |
| tc | 122 |

END

1. A messenger RNA (mRNA) encoding an intracellular lipid trafficker, wherein the mRNA comprises one or both of:
(i) an optimised codon;
(ii) a chemical modification,
when compared to a corresponding mRNA that does not comprise the optimised codon and/or chemical modification,
wherein the mRNA encodes an intracellular cholesterol trafficker selected from NPC intracellular cholesterol transporter 1 (NPC-1) and NPC intracellular cholesterol transporter 2 (NPC-2).

2. The mRNA according to claim 1, wherein the mRNA encodes NPC-1.

3. The mRNA according to claim 1 or 2, wherein the mRNA encoding NPC-1 comprises a sequence having at least 70%, at least 75%, at least 80%, at least 85%, at least 90%, at least 95%, at least 96%, at least 97%, at least 98%, at least 99%, at least 99.5% or at least 99.8% identity to a sequence set forth as SEQ ID NO: 2 or 8.

4. The mRNA according to claim 3, wherein the mRNA encoding NPC-1 comprises a sequence set forth as SEQ ID NO: 2.

5. The mRNA according to claim 1, wherein the mRNA encoding NPC-2 comprises a sequence having at least 70%, at least 75%, at least 80%, at least 85%, at least 90%, at least 95%, at least 96%, at least 97%, at least 98%, at least 99%, at least 99.5% or at least 99.8% identity to a sequence set forth as SEQ ID NO: 9.

6. The mRNA according to any one of claims 1 to 5, wherein the lipid comprises cholesterol.

7. The mRNA according to claim 6, wherein the cholesterol is unesterified cholesterol.

8. The mRNA according to claim 7, wherein the lipid comprises sphingomyelin, a glycosphingolipid or bis(monoacylglycerol) phosphate.

9. The mRNA according to claim 8, wherein the lipid comprises glycosphingolipids selected from the group consisting of cerebrosides, gangliosides and globosides.

10. The mRNA according to any one of claims 1 to 9, wherein the mRNA comprises an optimised codon and a chemical modification.

11. The mRNA according to claim 10, wherein the chemical modification increases mRNA stability and/or mRNA translation when compared to a mRNA without the chemical modification.

12. The mRNA according to claim 10 or 11, wherein the chemical modification is a nucleoside modification.

13. The mRNA according to claim 12, wherein the nucleoside modification is a modified uracil.

14. The mRNA according to claim 13, wherein the uracil modification is selected from the group consisting of pseudouridine (ψ), pyridin-4-one ribonucleoside, 5-aza-uridine, 6-aza-uridine, 2-thio-5-aza-uridine, 2-thio-uridine (s²U), 4-thio-uridine (s⁴U), 4-thiopseudouridine, 2-thio-pseudouridine, 5-hydroxy-uridine (ho⁵U), 5-aminoallyl-uridine, 5-halo-uridine (e.g., 5-iodo-uridine or 5-bromo-uridine), 3-methyluridine (m³U), 5-methoxy-uridine (mo⁵U), 5-methoxy-uridine triphosphate (5moUTP), uridine 5-oxyacetic acid (cmo⁵U), uridine 5-oxyacetic acid methyl ester (mcmo⁵U), 5-carboxymethyl-uridine (cm⁵U), 1-carboxymethyl-pseudouridine, 5-carboxyhydroxymethyl-uridine (chm⁵U), 5-carboxyhydroxymethyl-uridine methyl ester (mchm⁵U), 5-methoxycarbonylmethyl-uridine (mcm⁵U), 5-methoxycarbonylmethyl-2-thio-uridine (mcm⁵s²U), 5-aminomethyl-2-thio-uridine (nm⁵s²U), 5-methylaminomethyl-uridine (mnm⁵U), 5-methylaminomethyl-2-thio-uridine (mnm⁵s²U), 5-methylaminomethyl-2-seleno-uridine (mnm⁵se²U), 5-carbamoylmethyl-uridine (ncm⁵U), 5-carboxymethylaminomethyl-uridine (cmnm⁵U), 5-carboxymethylaminomethyl-2-thio-uridine (cmnm⁵s²U), 5-propynyluridine, 1-propynyl-pseudouridine, 5-taurinomethyl-uridine (tm⁵U), 1-taurinomethyl-pseudouridine, 5-taurinomethyl-2-thio-uridine (tm⁵s²U), 1-taurinomethyl-4-thio-pseudouridine, 5-methyl-uridine (m⁵U, i.e., having the nucleobase deoxythymine), 1-methylpseudouridine (m¹ψ), 5-methyl-2-thiouridine (m⁵s²U), 1-methyl-4-thio-pseudouridine (m¹s⁴ψ), 4-thio-1-methyl-pseudouridine, 3-methyl-pseudouridine (m³ψ), 2-thio-1-methyl-pseudouridine, 1-methyl-1-deaza-pseudouridine, 2-thio-1-methyl-1-deaza-pseudouridine, dihydrouridine (D), dihydropseudouridine, 5,6-dihydrouridine, 5-methyldihydrouridine (m⁵D), 2-thio-dihydrouridine, 2-thio-dihydropseudouridine, 2-methoxy-uridine, 2-methoxy-4-thio-uridine, 4-methoxy-pseudouridine, 4-methoxy-2-thio-pseudouridine, N1-methyl-pseudouridine (also known as 1-methylpseudouridine (m¹ψ), 3-(3-amino-3-carboxypropyl) uridine (acp³U), 1-methyl-3-(3-amino-3-carboxypropyl) pseudouridine (acp³ψ), 5-(isopentenylaminomethyl) uridine (inm⁵U), 5-(isopentenylaminomethyl)-2-thio-uridine (inm⁵s²U), a-thio-uridine, 2′-O-methyl-uridine (Um), 5,2′-O-dimethyl-uridine (m⁵Um), 2′-O-methyl-pseudouridine (ψm), 2-thio-2′-O-methyl-uridine (s²Um), 5-methoxycarbonylmethyl-2′-O-methyl-uridine (mcm⁵Um), 5-carbamoylmethyl-2′-O-methyl-uridine (ncm⁵Um), 5-carboxymethylaminomethyl-2′-O-methyluridine (cmnm⁵Um), 3,2′-O-dimethyl-uridine (m³Um), 5-(isopentenylaminomethyl)-2′-O-methyl-uridine (inm⁵Um), 1-thio-uridine, deoxythymidine, 2′-F-arauridine, 2′-F-uridine, 2′-OH-ara-uridine, 5-(2-carbomethoxyvinyl) uridine, and 5-[3-(1-E-propenylamino)]uridine.

15. The mRNA according to claim 14, wherein the uracil modification is 5-methoxyuridine triphosphate (5moUTP) or N1-methylpseudouridine 5′-triphosphate (M1ψTP), preferably M1ψTP.

16. The mRNA according to any one of claims 13 to 15, wherein each uracil is modified to 5-methoxyuridine triphosphate (5moUTP) or N1-methylpseudouridine 5′-triphosphate (M1 ψTP), preferably M1ψTP.

17. The mRNA according to claim 12, wherein the nucleoside modification is a modified cytosine.

18. The mRNA according to claim 17, wherein the cytosine modification is selected from the group consisting of 5-aza-cytidine, 6-aza-cytidine, pseudoisocytidine, 3-methyl-cytidine (m³C), N4-acetyl-cytidine (ac⁴C), 5-formyl-cytidine (f⁵C), N4-methyl-cytidine (m⁴C), 5-methyl-cytidine (m⁵C), 5-methy-lcytidine 5′-triphosphate (5mCTP), 5-halo-cytidine (e.g., 5-iodo-cytidine), 5-hydroxymethyl-cytidine (hm⁵C), 1-methyl-pseudoisocytidine, pyrrolo-cytidine, pyrrolo-pseudoisocytidine, 2-thio-cytidine (s²C), 2-thio-5-methyl-cytidine, 4-thio-pseudoisocytidine, 4-thio-1-methyl-pseudoisocytidine, 4-thio-1-methyl-1-deaza-pseudoisocytidine, 1-methyl-1-deaza-pseudoisocytidine, zebularine, 5-aza-zebularine, 5-methyl-zebularine, 5-aza-2-thio-zebularine, 2-thiozebularine, 2-methoxy-cytidine, 2-methoxy-5-methyl-cytidine, 4-methoxy-pseudoisocytidine, 4-methoxy-1-methyl-pseudoisocytidine, lys idine (k₂C), a-thiocytidine, 2′-O-methyl-cytidine (Cm), 5,2′-O-dimethyl-cytidine (m⁵Cm), N4-acetyl-2′-O-methyl-cytidine (ac⁴Cm), N4,2′-O-dimethyl-cytidine (m⁴Cm), 5-formyl-2′-O-methyl-cytidine (f⁵Cm), N4,N4,2′-O-trimethyl-cytidine (m⁴₂Cm), 1-thio-cytidine, 2′-F-ara-cytidine, 2′-F-cytidine, and 2′-OH-ara-cytidine.

19. The mRNA according to claim 18, wherein the nucleoside modification is 5-methylcytidine 5′-triphosphate (5mCTP).

20. The mRNA according to any one of claims 1 to 19, wherein the optimised codon increases mRNA stability and/or mRNA translation when compared to a mRNA without the optimised codon.

21. The mRNA according to claim 20, wherein the optimised codon increases guanine (G) and/or cytosine (C) codon content.

22. The mRNA according to claim 20 or 21, wherein the optimised codon comprises substituting adenine (A) or uracil (U) containing codons with codons enriched in guanine (G) or cytosine (C), preferably at the third base of a codon.

23. The mRNA according to claim 22, wherein uracil is modified to M1ψTP and codons comprising A or U, preferably at the third base, are substituted with codons enriched in guanine (G) or cytosine (C).

24. The mRNA according to claim 23, wherein the chemical modifications and optimised codons increase mRNA translation by between about 900-1100 fold, preferably about 1000 fold, when compared to a mRNA without the chemical modifications and optimised codons.

25. The mRNA according to any one of claims 1 to 24, wherein the mRNA comprises a cap, 5′UTR, coding sequence, a 3′UTR and a poly A tail.

26. The mRNA according to claim 25, wherein the cap comprises a cap 1 mRNA structure, preferably according to the sequence AG.

27. The mRNA according to claim 25 or 26, wherein the 5′UTR and 3′UTR comprise sequences that increase mRNA stability and/or polypeptide expression.

28. The mRNA according to any one of claims 25 to 27, wherein the 5′UTR is an elongation factor, preferably elongation factor 1-beta (EEF1B2), more preferably according to the sequence set forth in SEQ ID NO: 1.

29. The mRNA according to any one of claims 25 to 28, wherein the 3′UTR is alpha-1 globin, preferably according to the sequence set forth in SEQ ID NO: 3.

30. The mRNA according to any one of claims 25 to 29, wherein the poly A tail comprises between 25 to 200 nucleotides, between 50 to 150 nucleotides, between 80 to 120 nucleotides, preferably about 100 nucleotides.

31. The mRNA according to claim 30, comprising a sequence having at least 70%, at least 75%, at least 80%, at least 85%, at least 90%, at least 95%, at least 96%, at least 97%, at least 98%, at least 99%, at least 99.5% at least 99.8% or 100% identity to a sequence set forth as SEQ ID NO: 4.

32. The mRNA according to any one of claims 25 to 31, comprising a sequence having at least 70%, at least 75%, at least 80%, at least 85%, at least 90%, at least 95%, at least 96%, at least 97%, at least 98%, at least 99%, at least 99.5% at least 99.8% or 100% identity to a sequence set forth as SEQ ID NO: 5.

33. A mammalian cell, preferably a cell of the central nervous system, liver, spleen or lungs, comprising the mRNA according to any one of claims 1 to 32.

34. A pharmaceutical composition comprising the mRNA according to any one of claims 1 to 32 and a pharmaceutically acceptable carrier, diluent or excipient.

35. A kit comprising the mRNA according to any one of claims 1 to 32 or the pharmaceutical composition according to claim 34.

36. A method of treating or preventing a lysosomal storage disease in a subject in need thereof, comprising administering a therapeutically effective amount of the mRNA according to any one of claims 1 to 32 or the pharmaceutical composition according to claim 34 to the subject, thereby treating or preventing the lysosomal storage disease in the subject in need thereof.

37. Use of a therapeutically effective amount of the mRNA according to any one of claims 1 to 32 or the pharmaceutical composition according to claim 34 in the preparation of a medicament for treating or preventing a lysosomal storage disease.

38. The mRNA according to any one of claims 1 to 32 or the pharmaceutical composition according to claim 34, for use in treating or preventing a lysosomal storage disease.

39. The method according to claim 36, the use according to claim 37, or the mRNA for use according to claim 38, wherein the lysosomal storage disease is associated with impaired intracellular lipid trafficking.

40. The method, the use or the mRNA for use according to claim 39, wherein the lipid comprises cholesterol, sphingomyelin, a glycosphingolipid, optionally selected from the group consisting of cerebrosides, gangliosides and globosides, and/or bis(monoacylglycerol) phosphate.

41. The method, the use or the mRNA for use according to claim 39 or 40, wherein the lysosomal storage disease is associated with a mutation to an intracellular cholesterol trafficker, preferably selected from NPC intracellular cholesterol transporter 1 (NPC-1) or NPC intracellular cholesterol transporter 2 (NPC-2), most preferably NPC-1.

42. The method, the use or the mRNA for use according to claim 41, wherein the mutation to NPC-1 includes a mutation selected from the group consisting of Gly248Val, Met1142Thr, Ile1061Thr and Arg404Trp.

43. The method according to any one of claims 36 or 39 to 42, the use according to any one of claims 37, or 39 to 42 or the mRNA for use according to any one of claims 38, or 39 to 42, wherein the lysosomal storage disease is Niemann-Pick Type C disease.

44. The method according to any one of claims 36 or 39 to 43, the use according to any one of claims 37, or 39 to 43 or the mRNA for use according to any one of claims 38 to 43, wherein the treatment further comprises administering an inhibitor of glucosylceramide synthase, an inducer of heat shock proteins, a cyclic oligosaccharide or hydroxypropyl-beta-cyclodextrin.

45. The method according to any one of claims 36 or 39 to 43, the use according to any one of claims 37, or 39 to 43 or the mRNA for use according to any one of claims 38 to 43, wherein the treatment restores intracellular lipid trafficking in the subject, preferably in cells of the central nervous system (e.g., glial cells and oligodendrocytes), liver (e.g., hepatocytes), spleen (e.g., reticular cells and lymphocytes) and/or lungs (e.g., alveolar epithelial cells), most preferably in cells of the central nervous system (e.g., glial cells and neuronal cells).

46. The method, the use or the mRNA for use according to claim 45, wherein intracellular lipid trafficking is restored within lysosomes of the cells.

47. The method according to any one of claims 36 or 39 to 43, the use according to any one of claims 37, or 39 to 43 or the mRNA for use according to any one of claims 38 to 43, wherein the treatment reduces lipid accumulation in lysosomes, preferably in lysosomes of cells of the central nervous system (e.g., glial cells and oligodendrocytes), liver (e.g., hepatocytes), spleen (e.g., reticular cells and lymphocytes) and/or lungs (e.g., alveolar epithelial cells), most preferably in cells of the central nervous system (e.g., glial cells and neuronal cells).

48. The method, the use or the mRNA for use according to claim 47, wherein the lipid comprises cholesterol, sphingomyelin, glycosphingolipid and/or bis(monoacylglycerol) phosphate.

49. The method, the use or the mRNA for use according to claim 48, wherein the cholesterol is unesterified cholesterol.

50. The method, the use or the mRNA for use according to any one of claims 47 to 49, wherein cholesterol esterification is increased in the subject.

51. The method according to any one of claims 36 or 39 to 43, the use according to any one of claims 37, or 39 to 43 or the mRNA for use according to any one of claims 38 to 43, wherein lysosomal size is reduced in cells of the central nervous system (e.g., glial cells and neuronal cells), liver (e.g., hepatocytes), spleen (e.g., reticular cells and lymphocytes) and/or lungs (e.g., alveolar epithelial cells).

52. The method according to any one of claims 36 or 39 to 43, the use according to any one of claims 37, or 39 to 43 or the mRNA for use according to any one of claims 38 to 43, wherein the mRNA or pharmaceutical composition is suitable for delivery to the liver, spleen, lungs and/or central nervous system of the subject.

53. The method, the use or the mRNA for use according to claim 52, wherein the mRNA or pharmaceutical composition is formulated in a lipid nanoparticle.

54. A process for identifying an mRNA that provides for increased polypeptide expression of an intracellular lipid trafficker comprising:
i) introducing the mRNA of any one of claims 1 to 32 into a cell under conditions sufficient for the expression of the polypeptide encoded by the mRNA within the cell,
ii) determining whether the level of the intracellular lipid trafficker is increased relative to a corresponding cell lacking the mRNA.

55. The process according to claim 54, wherein the intracellular lipid trafficker comprises a sequence having at least 70%, at least 75%, at least 80%, at least 85%, at least 90%, at least 95%, at least 96%, at least 97%, at least 98%, at least 99%, at least 99.5% or at least 99.8% to a sequence set forth as SEQ ID NOs: 2, 5, 8 or 9.

56. The process of claim 55, wherein the intracellular lipid trafficker comprises a sequence set forth as SEQ ID NO: 2 or 5.

57. A method of synthesis of a mRNA encoding an intracellular lipid trafficker comprising expressing a DNA polynucleotide encoding a mRNA according to any one of claims 1 to 32, thereby synthesising a mRNA encoding an intracellular lipid trafficker.

58. The method according to claim 57, further comprising introducing a chemical modification to the mRNA.

59. The method according to claim 57 or 58, wherein the DNA polynucleotide is provided in a plasmid.

60. mRNA obtained from the method of any one of claims 1 to 32.

61. A DNA polynucleotide for in vitro transcription of an mRNA according to any one of claims 1 to 32, optionally comprised within a plasmid.


SEQUENCE LISTING (13 sequences)

SEQ ID NO: 1 - RNA, 122 nt, Synthetic construct
  source 1..122: mol_type = mRNA
gcgcagttccgccggaagtggccccagcctcgaggccgggcgtcttcggtcatctccggcgcttctagggctggttcccgtcatcttcgggagccgtggagctctcggatacagccgacacc

SEQ ID NO: 2 - RNA, 3837 nt, Homo sapiens
  source 1..3837: mol_type = mRNA
atgaccgcccggggcctggccctgggcctgctgctgctgctgctgtgccccgcccaggtgttcagccagagctgcgtgtggtacggcgagtgcggcatcgcctacggcgacaagcggtacaactgcgagtacagcggcccccccaagcccctgcccaaggacggctacgacctggtgcaggagctgtgccccggcttcttcttcggcaacgtgagcctgtgctgcgacgtgcggcagctgcagaccctgaaggacaacctgcagctgcccctgcagttcctgagccggtgccccagctgcttctacaacctgctgaacctgttctgcgagctgacctgcagcccccggcagagccagttcctgaacgtgaccgccaccgaggactacgtggaccccgtgaccaaccagaccaagaccaacgtgaaggagctgcagtactacgtgggccagagcttcgccaacgccatgtacaacgcctgccgggacgtggaggcccccagcagcaacgacaaggccctgggcctgctgtgcggcaaggacgccgacgcctgcaacgccaccaactggatcgagtacatgttcaacaaggacaacggccaggcccccttcaccatcacccccgtgttcagcgacttccccgtgcacggcatggagcccatgaacaacgccaccaagggctgcgacgagagcgtggacgaggtgaccgccccctgcagctgccaggactgcagcatcgtgtgcggccccaagccccagccgccgccgccgccggccccctggaccatcctgggcctggacgccatgtacgtgatcatgtggatcacctacatggccttcctgctggtgttcttcggcgccttcttcgccgtgtggtgctaccggaagcggtacttcgtgagcgagtacacccccatcgacagcaacatcgccttcagcgtgaacgccagcgacaagggcgaggccagctgctgcgaccccgtgagcgccgccttcgagggctgcctgcggcggctgttcacccggtggggcagcttctgcgtgcggaaccccggctgcgtgatcttcttcagcctggtgttcatcaccgcctgcagcagcggcctggtgttcgtgcgggtgaccaccaaccccgtggacctgtggagcgcccccagcagccaggcccggctggagaaggagtacttcgaccagcacttcggccccttcttccggaccgagcagctgatcatccgggcccccctgaccgacaagcacatctaccagccctaccccagcggcgccgacgtgcccttcggcccccccctggacatccagatcctgcaccaggtgctggacctgcagatcgccatcgagaacatcaccgccagctacgacaacgagaccgtgaccctgcaggacatctgcctggcccccctgagcccctacaacaccaactgcaccatcctgagcgtgctgaactacttccagaacagccacagcgtgctggaccacaagaagggcgacgacttcttcgtgtacgccgactaccacacccacttcctgtactgcgtgcgggcccccgccagcctgaacgacaccagcctgctgcacgacccctgcctgggcaccttcggcggccccgtgttcccctggctggtgctgggcggctacgacgaccagaactacaacaacgccaccgccctggtgatcaccttccccgtgaacaactactacaacgacaccgagaagctgcagcgggcccaggcctgggagaaggagttcatcaacttcgtgaagaactacaagaaccccaacctgaccatcagcttcaccgccgagcggagcatcgaggacgagctgaaccgggagagcgacagcgacgtgttcaccgtggtgatcagctacgccatcatgttcctgtacatcagcctggccctgggccacatgaagagctgccggcggctgctggtggacagcaaggtgagcctgggcatcgccggcatcctgatcgtgctgagcagcgtggcctgcagcctgggcgtgttcagctacatcggcctgcccctgaccctgatcgtgatcgaggtgatccccttcctggtgctggccgtgggcgtggacaacatcttcatcctggtgcaggcctaccagcgggacgagcggctgcagggcgagaccctggaccagcagctgggccgggtgctgggcgaggtggcccccagcatgttcctgagcagcttcagcgagaccgtggccttcttcctgggcgccctgagcgtgatgcccgccgtgcacaccttcagcctgttcgccggcctggccgtgttcatcgacttcctgctgcagatcacctgcttcgtgagcctgctgggcctggacatcaagcggcaggagaagaaccggctggacatcttctgctgcgtgcggggcgccgaggacggcaccagcgtgcaggccagcgagagctgcctgttccggttcttcaagaacagctacagccccctgctgctgaaggactggatgcggcccatcgtgatcgccatcttcgtgggcgtgctgagcttcagcatcgccgtgctgaacaaggtggacatcggcctggaccagagcctgagcatgcccgacgacagctacatggtggactacttcaagagcatcagccagtacctgcacgccggcccccccgtgtacttcgtgctggaggagggccacgactacaccagcagcaagggccagaacatggtgtgcggcggcatgggctgcaacaacgacagcctggtgcagcagatcttcaacgccgcccagctggacaactacacccggatcggcttcgcccccagcagctggatcgacgactacttcgactgggtgaagccccagagcagctgctgccgggtggacaacatcaccgaccagttctgcaacgccagcgtggtggaccccgcctgcgtgcggtgccggcccctgacccccgagggcaagcagcggccccagggcggcgacttcatgcggttcctgcccatgttcctgagcgacaaccccaaccccaagtgcggcaagggcggccacgccgcctacagcagcgccgtgaacatcctgctgggccacggcacccgggtgggcgccacctacttcatgacctaccacaccgtgctgcagaccagcgccgacttcatcgacgccctgaagaaggcccggctgatcgccagcaacgtgaccgagaccatgggcatcaacggcagcgcctaccgggtgttcccctacagcgtgttctacgtgttctacgagcagtacctgaccatcatcgacgacaccatcttcaacctgggcgtgagcctgggcgccatcttcctggtgaccatggtgctgctgggctgcgagctgtggagcgccgtgatcatgtgcgccaccatcgccatggtgctggtgaacatgttcggcgtgatgtggctgtggggcatcagcctgaacgccgtgagcctggtgaacctggtgatgagctgcggcatcagcgtggagttctgcagccacatcacccgggccttcaccgtgagcatgaagggcagccgggtggagcgggccgaggaggccctggcccacatgggcagcagcgtgttcagcggcatcaccctgaccaagttcggcggcatcgtggtgctggccttcgccaagagccagatcttccagatcttctacttccggatgtacctggccatggtgctgctgggcgccacccacggcctgatcttcctgcccgtgctgctgagctacatcggccccagcgtgaacaaggccaagagctgcgccaccgaggagcggtacaagggcaccgagcgggagcggctgctgaacttctga

SEQ ID NO: 3 - RNA, 111 nt, Synthetic construct
  source 1..111: mol_type = mRNA
gctggagcctcggtggccatgcttcttgccccttgggcctccccccagcccctcctccccttcctgcacccgtacccccgtggtctttgaataaagtctgagtgggcggca

SEQ ID NO: 4 - RNA, 100 nt, Synthetic construct
  source 1..100: mol_type = mRNA
aaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 5 - RNA, 4172 nt, Synthetic construct
  source 1..4172: mol_type = mRNA
aggcgcagttccgccggaagtggccccagcctcgaggccgggcgtcttcggtcatctccggcgcttctagggctggttcccgtcatcttcgggagccgtggagctctcggatacagccgacaccatgaccgcccggggcctggccctgggcctgctgctgctgctgctgtgccccgcccaggtgttcagccagagctgcgtgtggtacggcgagtgcggcatcgcctacggcgacaagcggtacaactgcgagtacagcggcccccccaagcccctgcccaaggacggctacgacctggtgcaggagctgtgccccggcttcttcttcggcaacgtgagcctgtgctgcgacgtgcggcagctgcagaccctgaaggacaacctgcagctgcccctgcagttcctgagccggtgccccagctgcttctacaacctgctgaacctgttctgcgagctgacctgcagcccccggcagagccagttcctgaacgtgaccgccaccgaggactacgtggaccccgtgaccaaccagaccaagaccaacgtgaaggagctgcagtactacgtgggccagagcttcgccaacgccatgtacaacgcctgccgggacgtggaggcccccagcagcaacgacaaggccctgggcctgctgtgcggcaaggacgccgacgcctgcaacgccaccaactggatcgagtacatgttcaacaaggacaacggccaggcccccttcaccatcacccccgtgttcagcgacttccccgtgcacggcatggagcccatgaacaacgccaccaagggctgcgacgagagcgtggacgaggtgaccgccccctgcagctgccaggactgcagcatcgtgtgcggccccaagccccagccgccgccgccgccggccccctggaccatcctgggcctggacgccatgtacgtgatcatgtggatcacctacatggccttcctgctggtgttcttcggcgccttcttcgccgtgtggtgctaccggaagcggtacttcgtgagcgagtacacccccatcgacagcaacatcgccttcagcgtgaacgccagcgacaagggcgaggccagctgctgcgaccccgtgagcgccgccttcgagggctgcctgcggcggctgttcacccggtggggcagcttctgcgtgcggaaccccggctgcgtgatcttcttcagcctggtgttcatcaccgcctgcagcagcggcctggtgttcgtgcgggtgaccaccaaccccgtggacctgtggagcgcccccagcagccaggcccggctggagaaggagtacttcgaccagcacttcggccccttcttccggaccgagcagctgatcatccgggcccccctgaccgacaagcacatctaccagccctaccccagcggcgccgacgtgcccttcggcccccccctggacatccagatcctgcaccaggtgctggacctgcagatcgccatcgagaacatcaccgccagctacgacaacgagaccgtgaccctgcaggacatctgcctggcccccctgagcccctacaacaccaactgcaccatcctgagcgtgctgaactacttccagaacagccacagcgtgctggaccacaagaagggcgacgacttcttcgtgtacgccgactaccacacccacttcctgtactgcgtgcgggcccccgccagcctgaacgacaccagcctgctgcacgacccctgcctgggcaccttcggcggccccgtgttcccctggctggtgctgggcggctacgacgaccagaactacaacaacgccaccgccctggtgatcaccttccccgtgaacaactactacaacgacaccgagaagctgcagcgggcccaggcctgggagaaggagttcatcaacttcgtgaagaactacaagaaccccaacctgaccatcagcttcaccgccgagcggagcatcgaggacgagctgaaccgggagagcgacagcgacgtgttcaccgtggtgatcagctacgccatcatgttcctgtacatcagcctggccctgggccacatgaagagctgccggcggctgctggtggacagcaaggtgagcctgggcatcgccggcatcctgatcgtgctgagcagcgtggcctgcagcctgggcgtgttcagctacatcggcctgcccctgaccctgatcgtgatcgaggtgatccccttcctggtgctggccgtgggcgtggacaacatcttcatcctggtgcaggcctaccagcgggacgagcggctgcagggcgagaccctggaccagcagctgggccgggtgctgggcgaggtggcccccagcatgttcctgagcagcttcagcgagaccgtggccttcttcctgggcgccctgagcgtgatgcccgccgtgcacaccttcagcctgttcgccggcctggccgtgttcatcgacttcctgctgcagatcacctgcttcgtgagcctgctgggcctggacatcaagcggcaggagaagaaccggctggacatcttctgctgcgtgcggggcgccgaggacggcaccagcgtgcaggccagcgagagctgcctgttccggttcttcaagaacagctacagccccctgctgctgaaggactggatgcggcccatcgtgatcgccatcttcgtgggcgtgctgagcttcagcatcgccgtgctgaacaaggtggacatcggcctggaccagagcctgagcatgcccgacgacagctacatggtggactacttcaagagcatcagccagtacctgcacgccggcccccccgtgtacttcgtgctggaggagggccacgactacaccagcagcaagggccagaacatggtgtgcggcggcatgggctgcaacaacgacagcctggtgcagcagatcttcaacgccgcccagctggacaactacacccggatcggcttcgcccccagcagctggatcgacgactacttcgactgggtgaagccccagagcagctgctgccgggtggacaacatcaccgaccagttctgcaacgccagcgtggtggaccccgcctgcgtgcggtgccggcccctgacccccgagggcaagcagcggccccagggcggcgacttcatgcggttcctgcccatgttcctgagcgacaaccccaaccccaagtgcggcaagggcggccacgccgcctacagcagcgccgtgaacatcctgctgggccacggcacccgggtgggcgccacctacttcatgacctaccacaccgtgctgcagaccagcgccgacttcatcgacgccctgaagaaggcccggctgatcgccagcaacgtgaccgagaccatgggcatcaacggcagcgcctaccgggtgttcccctacagcgtgttctacgtgttctacgagcagtacctgaccatcatcgacgacaccatcttcaacctgggcgtgagcctgggcgccatcttcctggtgaccatggtgctgctgggctgcgagctgtggagcgccgtgatcatgtgcgccaccatcgccatggtgctggtgaacatgttcggcgtgatgtggctgtggggcatcagcctgaacgccgtgagcctggtgaacctggtgatgagctgcggcatcagcgtggagttctgcagccacatcacccgggccttcaccgtgagcatgaagggcagccgggtggagcgggccgaggaggccctggcccacatgggcagcagcgtgttcagcggcatcaccctgaccaagttcggcggcatcgtggtgctggccttcgccaagagccagatcttccagatcttctacttccggatgtacctggccatggtgctgctgggcgccacccacggcctgatcttcctgcccgtgctgctgagctacatcggccccagcgtgaacaaggccaagagctgcgccaccgaggagcggtacaagggcaccgagcgggagcggctgctgaacttctgagctggagcctcggtggccatgcttcttgccccttgggcctccccccagcccctcctccccttcctgcacccgtacccccgtggtctttgaataaagtctgagtgggcggcaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 6 - RNA, 1653 nt, Photinus pyralis
  source 1..1653: mol_type = mRNA
atggaggacgccaagaacatcaagaagggccccgcccccttctaccccctggaggacggcaccgccggcgagcagctgcacaaggccatgaagcggtacgccctggtgcccggcaccatcgccttcaccgacgcccacatcgaggtggacatcacctacgccgagtacttcgagatgagcgtgcggctggccgaggccatgaagcggtacggcctgaacaccaaccaccggatcgtggtgtgcagcgagaacagcctgcagttcttcatgcccgtgctgggcgccctgttcatcggcgtggccgtggcccccgccaacgacatctacaacgagcgggagctgctgaacagcatgggcatcagccagcccaccgtggtgttcgtgagcaagaagggcctgcagaagatcctgaacgtgcagaagaagctgcccatcatccagaagatcatcatcatggacagcaagaccgactaccagggcttccagagcatgtacaccttcgtgaccagccacctgccccccggcttcaacgagtacgacttcgtgcccgagagcttcgaccgggacaagaccatcgccctgatcatgaacagcagcggcagcaccggcctgcccaagggcgtggccctgccccaccggaccgcctgcgtgcggttcagccacgcccgggaccccatcttcggcaaccagatcatccccgacaccgccatcctgagcgtggtgcccttccaccacggcttcggcatgttcaccaccctgggctacctgatctgcggcttccgggtggtgctgatgtaccggttcgaggaggagctgttcctgcggagcctgcaggactacaagatccagagcgccctgctggtgcccaccctgttcagcttcttcgccaagagcaccctgatcgacaagtacgacctgagcaacctgcacgagatcgccagcggcggcgcccccctgagcaaggaggtgggcgaggccgtggccaagcggttccacctgcccggcatccggcagggctacggcctgaccgagaccaccagcgccatcctgatcacccccgagggcgacgacaagcccggcgccgtgggcaaggtggtgcccttcttcgaggccaaggtggtggacctggacaccggcaagaccctgggcgtgaaccagcggggcgagctgtgcgtgcggggccccatgatcatgagcggctacgtgaacaaccccgaggccaccaacgccctgatcgacaaggacggctggctgcacagcggcgacatcgcctactgggacgaggacgagcacttcttcatcgtggaccggctgaagagcctgatcaagtacaagggctaccaggtggcccccgccgagctggagagcatcctgctgcagcaccccaacatcttcgacgccggcgtggccggcctgcccgacgacgacgccggcgagctgcccgccgccgtggtggtgctggagcacggcaagaccatgaccgagaaggagatcgtggactacgtggccagccaggtgaccaccgccaagaagctgcggggcggcgtggtgttcgtggacgaggtgcccaagggcctgaccggcaagctggacgcccggaagatccgggagatcctgatcaaggccaagaagggcggcaagatcgccgtgtga

SEQ ID NO: 7 - RNA, 1988 nt, Synthetic construct
  source 1..1988: mol_type = mRNA
aggcgcagttccgccggaagtggccccagcctcgaggccgggcgtcttcggtcatctccggcgcttctagggctggttcccgtcatcttcgggagccgtggagctctcggatacagccgacaccatggaggacgccaagaacatcaagaagggccccgcccccttctaccccctggaggacggcaccgccggcgagcagctgcacaaggccatgaagcggtacgccctggtgcccggcaccatcgccttcaccgacgcccacatcgaggtggacatcacctacgccgagtacttcgagatgagcgtgcggctggccgaggccatgaagcggtacggcctgaacaccaaccaccggatcgtggtgtgcagcgagaacagcctgcagttcttcatgcccgtgctgggcgccctgttcatcggcgtggccgtggcccccgccaacgacatctacaacgagcgggagctgctgaacagcatgggcatcagccagcccaccgtggtgttcgtgagcaagaagggcctgcagaagatcctgaacgtgcagaagaagctgcccatcatccagaagatcatcatcatggacagcaagaccgactaccagggcttccagagcatgtacaccttcgtgaccagccacctgccccccggcttcaacgagtacgacttcgtgcccgagagcttcgaccgggacaagaccatcgccctgatcatgaacagcagcggcagcaccggcctgcccaagggcgtggccctgccccaccggaccgcctgcgtgcggttcagccacgcccgggaccccatcttcggcaaccagatcatccccgacaccgccatcctgagcgtggtgcccttccaccacggcttcggcatgttcaccaccctgggctacctgatctgcggcttccgggtggtgctgatgtaccggttcgaggaggagctgttcctgcggagcctgcaggactacaagatccagagcgccctgctggtgcccaccctgttcagcttcttcgccaagagcaccctgatcgacaagtacgacctgagcaacctgcacgagatcgccagcggcggcgcccccctgagcaaggaggtgggcgaggccgtggccaagcggttccacctgcccggcatccggcagggctacggcctgaccgagaccaccagcgccatcctgatcacccccgagggcgacgacaagcccggcgccgtgggcaaggtggtgcccttcttcgaggccaaggtggtggacctggacaccggcaagaccctgggcgtgaaccagcggggcgagctgtgcgtgcggggccccatgatcatgagcggctacgtgaacaaccccgaggccaccaacgccctgatcgacaaggacggctggctgcacagcggcgacatcgcctactgggacgaggacgagcacttcttcatcgtggaccggctgaagagcctgatcaagtacaagggctaccaggtggcccccgccgagctggagagcatcctgctgcagcaccccaacatcttcgacgccggcgtggccggcctgcccgacgacgacgccggcgagctgcccgccgccgtggtggtgctggagcacggcaagaccatgaccgagaaggagatcgtggactacgtggccagccaggtgaccaccgccaagaagctgcggggcggcgtggtgttcgtggacgaggtgcccaagggcctgaccggcaagctggacgcccggaagatccgggagatcctgatcaaggccaagaagggcggcaagatcgccgtgtgagctggagcctcggtggccatgcttcttgccccttgggcctccccccagcccctcctccccttcctgcacccgtacccccgtggtctttgaataaagtctgagtgggcggcaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 8 - RNA, 3837 nt, Homo sapiens
  source 1..3837: mol_type = mRNA
atgaccgctcgcggcctggcccttggcctcctcctgctgctactgtgtccagcgcaggtgttttcacagtcctgtgtttggtatggagagtgtggaattgcatatggggacaagaggtacaattgcgaatattctggcccaccaaaaccattgccaaaggatggatatgacttagtgcaggaactctgtccaggattcttctttggcaatgtcagtctctgttgtgatgttcggcagcttcagacactaaaagacaacctgcagctgcctctacagtttctgtccagatgtccatcctgtttttataacctactgaacctgttttgtgagctgacatgtagccctcgacagagtcagtttttgaatgttacagctactgaagattatgttgatcctgttacaaaccagacgaaaacaaatgtgaaagagttacaatactacgtcggacagagttttgccaatgcaatgtacaatgcctgccgggatgtggaggccccctcaagtaatgacaaggccctgggactcctgtgtgggaaggacgctgacgcctgtaatgccaccaactggattgaatacatgttcaataaggacaatggacaggcaccttttaccatcactcctgtgttttcagattttccagtccatgggatggagcccatgaacaatgccaccaaaggctgtgacgagtctgtggatgaggtcacagcaccatgtagctgccaagactgctctattgtctgtggccccaagccccagcccccacctcctcctgctccctggacgatccttggcttggacgccatgtatgtcatcatgtggatcacctacatggcgtttttgcttgtgttttttggagcattttttgcagtgtggtgctacagaaaacggtattttgtctccgagtacactcccatcgatagcaatatagctttttctgttaatgcaagtgacaaaggagaggcgtcctgctgtgaccctgtcagcgcagcatttgagggctgcttgaggcggctgttcacacgctgggggtctttctgcgtccgaaaccctggctgtgtcattttcttctcgctggtcttcattactgcgtgttcgtcaggcctggtgtttgtccgggtcacaaccaatccagttgacctctggtcagcccccagcagccaggctcgcctggaaaaagagtactttgaccagcactttgggcctttcttccggacggagcagctcatcatccgggcccctctcactgacaaacacatttaccagccatacccttcgggagctgatgtaccctttggacctccgcttgacatacagatactgcaccaggttcttgacttacaaatagccatcgaaaacattactgcctcttatgacaatgagactgtgacacttcaagacatctgcttggcccctctttcaccgtataacacgaactgcaccattttgagtgtgttaaattacttccagaacagccattccgtgctggaccacaagaaaggggacgacttctttgtgtatgccgattaccacacgcactttctgtactgcgtacgggctcctgcctctctgaatgatacaagtttgctccatgacccttgtctgggtacgtttggtggaccagtgttcccgtggcttgtgttgggaggctatgatgatcaaaactacaataacgccactgcccttgtgattaccttccctgtcaataattactataatgatacagagaagctccagagggcccaggcctgggaaaaagagtttattaattttgtgaaaaactacaagaatcccaatctgaccatttccttcactgctgaacgaagtattgaagatgaactaaatcgtgaaagtgacagtgatgtcttcaccgttgtaattagctatgccatcatgtttctatatatttccctagccttggggcacatgaaaagctgtcgcaggcttctggtggattcgaaggtctcactaggcatcgcgggcatcttgatcgtgctgagctcggtggcttgctccttgggtgtcttcagctacattgggttgcccttgaccctcattgtgattgaagtcatcccgttcctggtgctggctgttggagtggacaacatcttcattctggtgcaggcctaccagagagatgaacgtcttcaaggggaaaccctggatcagcagctgggcagggtcctaggagaagtggctcccagtatgttcctgtcatccttttctgagactgtagcatttttcttaggagcattgtccgtgatgccagccgtgcacaccttctctctctttgcgggattggcagtcttcattgactttcttctgcagattacctgtttcgtgagtctcttggggttagacattaaacgtcaagagaaaaatcggctagacatcttttgctgtgtcagaggtgctgaagatggaacaagcgtccaggcctcagagagctgtttgtttcgcttcttcaaaaactcctattctccacttctgctaaaggactggatgagaccaattgtgatagcaatatttgtgggtgttctgtcattcagcatcgcagtcctgaacaaagtagatattggattggatcagtctctttcgatgccagatgactcctacatggtggattatttcaaatccatcagtcagtacctgcatgcgggtccgcctgtgtactttgtcctggaggaagggcacgactacacttcttccaaggggcagaacatggtgtgcggcggcatgggctgcaacaatgattccctggtgcagcagatatttaacgcggcgcagctggacaactatacccgaataggcttcgccccctcgtcctggatcgacgattatttcgactgggtgaagccacagtcgtcttgctgtcgagtggacaatatcactgaccagttctgcaatgcttcagtggttgaccctgcctgcgttcgctgcaggcctctgactccggaaggcaaacagaggcctcaggggggagacttcatgagattcctgcccatgttcctttcggataaccctaaccccaagtgtggcaaagggggacatgctgcctatagttctgcagttaacatcctccttggccatggcaccagggtcggagccacgtacttcatgacctaccacaccgtgctgcagacctctgctgactttattgacgctctgaagaaagcccgacttatagccagtaatgtcaccgaaaccatgggcattaacggcagtgcctaccgagtatttccttacagtgtgttttatgtcttctacgaacagtacctgaccatcattgacgacactatcttcaacctcggtgtgtccctgggcgcgatatttctggtgaccatggtcctcctgggctgtgagctctggtctgcagtcatcatgtgtgccaccatcgccatggtcttggtcaacatgtttggagttatgtggctctggggcatcagtctgaacgctgtatccttggtcaacctggtgatgagctgtggcatctccgtggagttctgcagccacataaccagagcgttcacggtgagcatgaaaggcagccgcgtggagcgcgcggaagaggcacttgcccacatgggcagctccgtgttcagtggaatcacacttacaaaatttggagggattgtggtgttggcttttgccaaatctcaaattttccagatattctacttcaggatgtatttggccatggtcttactgggagccactcacggattaatatttctccctgtcttactcagttacatagggccatcagtaaataaagccaaaagttgtgccactgaagagcgatacaaaggaacagagcgcgaacggcttctaaatttctag

SEQ ID NO: 9 - RNA, 525 nt, Homo sapiens
  source 1..525: mol_type = mRNA
atgcgtttcctggcagctacattcctgctcctggcgctcagcaccgctgcccaggccgaaccggtgcagttcaaggactgcggttctgtggatggagttataaaggaagtgaatgtgagcccatgccccacccaaccctgccagctgagcaaaggacagtcttacagcgtcaatgtcaccttcaccagcaatattcagtctaaaagcagcaaggccgtggtgcatggcatcctgatgggcgtcccagttccctttcccattcctgagcctgatggttgtaagagtggaattaactgccctatccaaaaagacaagacctatagctacctgaataaactaccagtgaaaagcgaatatccctctataaaactggtggtggagtggcaacttcaggatgacaaaaaccaaagtctcttctgctgggaaatcccagtacagatcgtaagtctatctgggggtgagagggcatgggtggagggaagaaagtggaggagaaatcagactgaaactaaatcagtgccataa

SEQ ID NO: 10 - protein, 1278 aa, Homo sapiens
MTARGLALGLLLLLLCPAQVFSQSCVWYGECGIAYGDKRYNCEYSGPPKPLPKDGYDLVQELCPGFFFGNVSLCCDVRQLQTLKDNLQLPLQFLSRCPSCFYNLLNLFCELTCSPRQSQFLNVTATEDYVDPVTNQTKTNVKELQYYVGQSFANAMYNACRDVEAPSSNDKALGLLCGKDADACNATNWIEYMFNKDNGQAPFTITPVFSDFPVHGMEPMNNATKGCDESVDEVTAPCSCQDCSIVCGPKPQPPPPPAPWTILGLDAMYVIMWITYMAFLLVFFGAFFAVWCYRKRYFVSEYTPIDSNIAFSVNASDKGEASCCDPVSAAFEGCLRRLFTRWGSFCVRNPGCVIFFSLVFITACSSGLVFVRVTTNPVDLWSAPSSQARLEKEYFDQHFGPFFRTEQLIIRAPLTDKHIYQPYPSGADVPFGPPLDIQILHQVLDLQIAIENITASYDNETVTLQDICLAPLSPYNTNCTILSVLNYFQNSHSVLDHKKGDDFFVYADYHTHFLYCVRAPASLNDTSLLHDPCLGTFGGPVFPWLVLGGYDDQNYNNATALVITFPVNNYYNDTEKLQRAQAWEKEFINFVKNYKNPNLTISFTAERSIEDELNRESDSDVFTVVISYAIMFLYISLALGHMKSCRRLLVDSKVSLGIAGILIVLSSVACSLGVFSYIGLPLTLIVIEVIPFLVLAVGVDNIFILVQAYQRDERLQGETLDQQLGRVLGEVAPSMFLSSFSETVAFFLGALSVMPAVHTFSLFAGLAVFIDFLLQITCFVSLLGLDIKRQEKNRLDIFCCVRGAEDGTSVQASESCLFRFFKNSYSPLLLKDWMRPIVIAIFVGVLSFSIAVLNKVDIGLDQSLSMPDDSYMVDYFKSISQYLHAGPPVYFVLEEGHDYTSSKGQNMVCGGMGCNNDSLVQQIFNAAQLDNYTRIGFAPSSWIDDYFDWVKPQSSCCRVDNITDQFCNASVVDPACVRCRPLTPEGKQRPQGGDFMRFLPMFLSDNPNPKCGKGGHAAYSSAVNILLGHGTRVGATYFMTYHTVLQTSADFIDALKKARLIASNVTETMGINGSAYRVFPYSVFYVFYEQYLTIIDDTIFNLGVSLGAIFLVTMVLLGCELWSAVIMCATIAMVLVNMFGVMWLWGISLNAVSLVNLVMSCGISVEFCSHITRAFTVSMKGSRVERAEEALAHMGSSVFSGITLTKFGGIVVLAFAKSQIFQIFYFRMYLAMVLLGATHGLIFLPVLLSYIGPSVNKAKSCATEERYKGTERERLLNF

SEQ ID NO: 11 - protein, 174 aa, Homo sapiens
MRFLAATFLLLALSTAAQAEPVQFKDCGSVDGVIKEVNVSPCPTQPCQLSKGQSYSVNVTFTSNIQSKSSKAVVHGILMGVPVPFPIPEPDGCKSGINCPIQKDKTYSYLNKLPVKSEYPSIKLVVEWQLQDDKNQSLFCWEIPVQIVSLSGGERAWVEGRKWRRNQTETKSVP

SEQ ID NO: 12 - DNA, 29 bp, Synthetic construct
  source 1..29: mol_type = other DNA
ccacgaatctaatacgactcactataagg

SEQ ID NO: 13 - DNA, 122 bp, Synthetic construct
  source 1..122: mol_type = other DNA
ttttttttttttttttttttttttttttttttttttttttttttttttttttttttttttttttttttttttttttttttttttttttttttttttttttgccgcccactcagactttattc